(19) United States
(12) Patent Application Publication
(10) Pub. No.: US 2023/0265381 A1
(43) Pub. Date: Aug. 24, 2023

(54) NOVEL CELL LINE

(71) Applicant: uniQure biopharma B.V., Amsterdam (NL)

(72) Inventors: David Johannes Francois DU PLESSIS, Amsterdam (NL); ANGGAKUSUMA, Amsterdam (NL)

(21) Appl. No.: 17/948,862

(22) Filed: Sep. 20, 2022

Related U.S. Application Data
(63) Continuation of application No. PCT/EP2021/058798, filed on Apr. 2, 2021.

(30) Foreign Application Priority Data
Apr. 2, 2020 (EP) 20167817.4

(51) Int. Cl. C12N 5/07; C07K 14/005; C12N 15/86; C07K 16/08

(52) U.S. Cl. CPC C12N 5/0601; C07K 14/005; C12N 15/86; C07K 16/081; C12N 2750/14143; C07K 2317/22

(57) ABSTRACT

The present invention relates to insect cell lines for the production of parvoviral gene therapy vectors. In particular the invention relates to stable insect cell lines with expression constructs for viral replicase proteins integrated into their genomes, which cell lines allow for high-yield, robust, and scalable production of heterologous parvoviral-related proteins and vectors.

### CROSS-REFERENCE TO RELATED APPLICATIONS

The present application is a continuation application of PCT/EP2021/058798 filed Apr. 2, 2021, which claims priority to EP 20167817.4 filed Apr. 2, 2020, the entire contents of both which are incorporated herein by reference.

### SEQUENCE LISTING

The instant application contains a Sequence Listing which has been submitted in XML format via Patent Center and is hereby incorporated by reference in its entirety. Said XML copy, created on Dec. 21, 2022, is named ST26_069818-0930.xml and is 262,270 bytes.

### FIELD OF THE INVENTION

The present invention relates to the fields of medicine, molecular biology, and gene therapy. The invention relates to production of proteins in cells whereby repeated imperfect palindromic/homologous repeat sequences are used in baculoviral vectors. In particular, the invention relates to the production of parvoviral vectors that may be used in gene therapy, and, to improvements in expression of the viral replicase (Rep) proteins that increase the productivity of parvoviral vectors.

### BACKGROUND OF THE INVENTION

The baculovirus expression system is well known for its use as a eukaryotic cloning and expression vector (King, L. A., and R. D. Possee, 1992, “The baculovirus expression system”, Chapman and Hall, United Kingdom; O′Reilly, D. R., et al., 1992. Baculovirus Expression Vectors: A Laboratory Manual. New York: W. H. Freeman). Advantages of the baculovirus expression system are, among others, that the expressed proteins are almost always soluble, correctly folded and biologically active. Further advantages include high protein expression levels, faster production, suitability for expression of large proteins and suitability for large-scale production. However, during large-scale or continuous production of heterologous proteins using the baculovirus system in insect cell bioreactors, the instability of production levels, also known as the passage effect, is a major obstacle. This effect is, at least in part, due to recombination between repeated homologous sequences in the baculoviral DNA.

The baculovirus expression system has also successfully been used for the production of recombinant adeno-associated virus (AAV) vectors (Urabe et al., 2002, Hum. Gene Ther. 13: 1935-1943; U.S. Pat. No. 6,723,551 and US 20040197895). AAV may be considered as one of the most promising viral vectors for human gene therapy. AAV has the ability to efficiently infect dividing as well as non-dividing human cells, and most importantly, even though AAV is present in many humans it has never been associated with any disease. In view of these advantages, recombinant adeno-associated virus (rAAV) is being evaluated in a diversity of gene therapy clinical trials, including trials for hemophilia B, malignant melanoma, cystic fibrosis, hyperlipoproteinemia type I and other diseases.

For large scale production of AAV, mammalian production systems are known to be less well suited, particularly as scale up will require a lot of bioreactor space. To overcome the problems in scaling up mammalian productions systems for AAV, Urabe et al. (2002, supra) developed an AAV production system in insect cells. To enable production of AAV in insect cells some modifications were necessary in order to achieve the correct stoichiometry of the three AAV capsid proteins (VP1, VP2 and VP3), which relies on a combination of alternate usage of two splice acceptor sites and the suboptimal utilization of an ACG initiation codon for VP2 that is not accurately reproduced by insect cells. To mimic the correct stoichiometry of the capsid proteins in insect cells, Urabe et al. (2002, supra) use a construct that is transcribed into a single polycistronic messenger that is able to express all three VP proteins without requiring splicing and wherein the most upstream initiator codon is replaced by the suboptimal initiator codon ACG. WO2007/046703 discloses further improvement of the infectivity of baculovirus-produced rAAV vectors achieved by further optimisation of the stoichiometry of AAV capsid proteins as produced in insect cells.

For expression of the AAV Rep proteins in the AAV baculovirus expression system, as initially developed by Urabe et al. (2002, supra), a recombinant baculovirus construct is used that harbours two independent Rep expression units (one for Rep78 and one for Rep52), each under the control of a separate baculovirus promoter, the ΔIE1 and polH promoters, respectively. However, Kohlbrenner et al. (2005, Mol. Ther. 12: 1217-25; WO 2005/072364) reported that the baculovirus construct for expression of the two Rep proteins, as used by Urabe et al., suffers from an inherent instability. By splitting the palindromic orientation of the two Rep genes in Urabe's original vector and designing two separate baculovirus vectors for expressing Rep52 and Rep78, Kohlbrenner et al. (2005, supra) increased the passaging stability of the vector. However, despite the consistent expression of Rep78 and Rep52 from the two independent baculovirus-Rep constructs in insect cells over at least 5 passages, rAAV vector yield is 5 to 10-fold lower as compared to the original baculovirus-Rep construct designed by Urabe et al. (2002, supra).

In application WO2007/148971 the present inventors have significantly improved the stability of rAAV vector production in insect cells by using a single coding sequence for the Rep78 and Rep52 proteins wherein a suboptimal initiator codon is used for the Rep78 protein that is partially skipped by the scanning ribosomes to allow for initiation of translation to also occur further downstream at the initiation codon of the Rep52 protein. In WO 2009/014445 the stability of rAAV vector production in insect cells was again further improved by employing separate expression cassettes for the Rep52 and Rep78, wherein the repeated coding sequences differ in codon bias to reduce homologous recombination.

International patent application WO 2007/084773 discloses a method of rAAV production in insect cells, wherein the production of infectious viral particles is increased by supplementing VP1 relative to VP2 and VP3. Supplementation can be affected by introducing into the insect cell a capsid vector comprising nucleotide sequences expressing VP1, VP2 and VP3 and additionally introducing into the insect cell nucleotide sequences expressing VP1, which may be either on the same capsid vector or on a different vector.

In 2009, Aslanidi et al. (Proc Natl Acad Sci U S A. 2009;106(13):5059-64) created an Sf9-based Rep-Cap packaging cell line that could produce AAV at 10⁵ genome copies (GC) per cell upon a single inoculation of baculovirus (Bac) harbouring the AAV ITR and transgene of interest

(Trans). This system, referred to as the OneBac platform, was considered to be suitable for the scale up of AAV production (Mietzsch, et al., 2014; Mietzsch, et al., 2017). However, Mietzsch et al. (2015) further optimized this platform to produce multiple AAV serotypes with low host-DNA false packaging. In a recent study, Wu et al. (2019) have shown that the OneBac platform can be more versatile and flexible by fusing the Cap gene together with the ITR-transgene-ITR (Cap-Trans) inside the baculovirus vector genome while maintaining the inducible Rep gene integrated within the packaging Rep Sf9 cells. All these experiments have shown the value of the OneBac platform and at the same time the necessity and possibility of improvement.

There is, therefore, still a need for further improvement in large scale (commercial) production of parvoviral vectors in cells, especially to overcome limitation associated with process robustness. Thus it is an object of the present invention to provide for means and methods that allow for high-yield, robust, and scalable production of heterologous parvoviral-related proteins and vectors.

### SUMMARY OF THE INVENTION

In a first aspect, the invention pertains to an insect cell comprising integrated into the genome of the cell: i) a first promoter operably linked to a nucleotide sequence encoding an mRNA, translation of which in the cell produces at least one of parvoviral Rep 78 and 68 proteins; ii) a second promoter operably linked to a nucleotide sequence encoding an mRNA, translation of which in the cell produces at least one of parvoviral Rep 52 and 40 proteins; and iii) at least one enhancer element that is operably linked to the first and second promoters, wherein the at least one enhancer element is dependent on a transcriptional transregulator, and wherein introduction of the transcriptional transregulator into the cell induces transcription from the first and second promoters. Preferably in the insect cell of the invention, the first and second promoters are baculoviral promoters, the transcriptional transregulator is a baculoviral immediate-early protein (IE1) or its splice variant (1E0) and the transcriptional transregulator-dependent enhancer element is a baculoviral homologous region (hr) enhancer element, wherein preferably the baculovirus is Autographa califomica multicapsid nucleopolyhedrovirus. Preferably, in the insect cell of the invention, the hr enhancer element is an hr enhancer element other than hr2-0.9, wherein preferably the hr enhancer element preferably comprises at least one copy of the hr 28-mer sequence CTTTACGAGTAGAATTCTACGCGTAAAA (SEQ ID NO. 32) and/or at least one copy of a of a sequence of which at least 20, 21, 22, 23, 24, 25, 26, or 27 nucleotides are identical to sequence CTTTACGAGTAGAATTCTACGCGTAAAA (SEQ ID NO. 32) and which binds to a baculoviral IE1 protein, and wherein the hr enhancer element, when operably linked to an expression cassette comprising a reporter gene operably linked to the polH promoter, a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and, b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element, More preferably, the hr enhancer element is selected from the group consisting of hr1, hr3, hr4b and hr5, of which hr4b and hr5 are preferred, of which hr4b is most preferred.

In the insect cell according to the invention, the first and second promoters are preferably distinct, wherein, more preferably the first promoter is a delayed early baculoviral promoter and the second promoter is a late or very late baculovirus promoter, most preferably the first promoter is the 39k promoter and the second promoter is selected from the group consisting of the polH, p10, p6.9 and pSel120 promoters.

In a preferred embodiment of the insect cell of the invention, the at least one of parvoviral Rep 52 and 40 proteins and the at least one of parvoviral Rep 78 and 68 proteins have a common amino acid sequence that is at least 90% identical, while the nucleotide sequence encoding the common amino acid sequence in the mRNA for the at least one of parvoviral Rep 52 and 40 proteins has less than 95, 90, 85, 80, 75, 70, 65 or 60% sequence identity with the nucleotide sequence encoding the common the amino acid sequence in the mRNA for the at least one of parvoviral Rep 78 and 68 proteins, wherein preferably the codon usage in the nucleotide sequence encoding the common the amino acid sequence in the mRNA for the at least one of parvoviral Rep 52 and 40 proteins, is more adapted to the codon usage bias of the insect cell than codon usage in the nucleotide sequence encoding the common the amino acid sequence in the mRNA for the at least one of parvoviral Rep 78 and 68 proteins.

In another preferred embodiment of the insect cell of the invention, the nucleotide sequence encoding the mRNA for the at least one of parvoviral Rep 78 and 68 proteins comprises a modification that affects a reduced steady state level of the at least one of parvoviral Rep 78 and 68 proteins, preferably the at least one of parvoviral Rep78 and 68 proteins comprises an open reading frame that starts with a suboptimal translation initiation codon, wherein more preferably, the suboptimal translation initiation codon is selected from ACG, CTG, TTG, GTG and ATT, of which ACG is most preferred.

Preferably in the insect cell according to the invention, the first and second promoters are integrated in the cell's genome in opposite directions of transcription and wherein the at least one enhancer element is present in between the first and second promoters, wherein more preferably two enhancer elements are present in between the first and second promoters.

An insect cell according to the invention further preferably comprises: a) a nucleotide sequence comprising parvoviral capsid protein coding sequences operably linked to a third promoter for expression in the insect cell; b) a nucleotide sequence comprising a transgene that is flanked by at least one parvoviral inverted terminal repeat sequence; and, c) a nucleotide sequence comprising an expression cassette for expression of the transcriptional transregulator, wherein preferably, the nucleotide sequences of at least one of a) and b) is comprised in a baculoviral vector, wherein, more preferably the nucleotide sequences of at least one of a), b) and c) are comprised in the baculoviral vector comprising the expression cassette for expression of the transcriptional transregulator. In a preferred embodiment, the first promoter is active before the third promoter.

In a preferred embodiment of the insect cell according to the invention, the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an adeno associated virus (AAV).

In a preferred embodiment of the insect cell according to the invention, the preferred cap-coding sequences comprise at least CAP AAV2/5 (SEQ ID 29) or AAV5 (SEQ ID 30).

In a second aspect, the invention relates to a method for producing a recombinant parvoviral virion comprising the steps of: a) culturing an insect cell as defined hereinabove; b) providing the cell cultured in a) with the nucleotide sequences as defined hereinabove; and, c) recovery of the recombinant parvoviral virion. Preferably in the method of the invention, recovery of the recombinant parvoviral virion in step c) comprises at least one of affinity-purification of the virion using an immobilised anti-parvoviral antibody, preferably a single chain camelid antibody or a fragment thereof, and filtration over a filter having a nominal pore size of 30-70 nm.

In a third aspect, the invention pertains to a kit of parts comprising at least an insect cell as defined hereinabove and a baculoviral vector and/or the nucleotide sequences as hereinabove.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the method.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . ,etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which may be determined as genome copies per kilogram (GC/kg). Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6X sodium chloride/sodium citrate (SSC) at about 45° C., followed by one or more washes in 1 X SSC, 0.1% SDS at about 50° C., preferably at about 55° C., preferably at about 60° C. and even more preferably at about 65° C.

Highly stringent conditions include, for example, hybridization at about 68° C. in 5× SSC/5× Denhardt's solution/1.0% SDS and washing in 0.2× SSC0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA tail, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

### DETAILED DESCRIPTION OF THE INVENTION

The present inventors have set out to develop improved packaging insect cell lines and vector systems for production of recombinant parvoviral vectors. In particular, the inventors have improved control of the inducible expression of Rep genes stably integrated in insect cell lines by providing means for reducing leaky expression under non-induced conditions while maintaining strong expression under induced conditions. Such insect cells are also referred to as iRep cells, or simply iRep. In addition the inventors have optimised the expression kinetics and ratio among the various parvoviral, e.g. AAV, structural and non-structural proteins, to further improve the robustness, yield and quality of vector output from a production platform, especially using the baculovirus and insect cell platform.

The vector quality is strongly related to the ratio between full virions vs empty virions, which contributes to potency of the vector itself. The term “full virion” refers to a virion particle that comprises parvoviral structural capsid proteins (VP1, VP2 and VP3) encapsulating the transgene DNA flanked by inverted terminal repeat (ITR) sequences. The term “empty virion” refers to a virion particle that does not comprise the parvoviral genomic material. In a preferred embodiment of the invention, the full virion vs empty virion ratio is at least 1:50, more preferably at least 1:10, or at least 1:5, or at least 1:2 and even more preferably at least 1:1. Even more preferably, no empty virions can be detected, and most preferably no empty virions are present. The person skilled in the art will know how to determine the full virion vs empty virion ratio, for example by dividing gene copy number by total particle with assembled AAV capsid number (or total assembled capsid:genome copy number), since per virion there will be only one genome copy present. The skilled artisan will know how to determine such ratios. For example, the ratio of empty virions vs. total capsids may be determined by dividing the amount of genome copies (i.e. genome copy number) by the amount of total parvoviral particles (i.e. number of parvoviral particles), wherein the amount of genome copies per ml is measured by quantitative PCR and the amount of total parvoviral particles per ml is measured with an enzyme immunoassay, e.g. from Progen.

### Insect Cell

In one aspect, there is provided an insect cell comprising integrated into the genome of the cell: i) a first promoter operably linked to a nucleotide sequence encoding an mRNA, translation of which in the cell produces only at least one of parvoviral Rep 78 and 68 proteins; ii) a second promoter operably linked to a nucleotide sequence encoding an mRNA, translation of which in the cell produces only at least one of parvoviral Rep 52 and 40 proteins; and iii) at least one enhancer element that is operably linked to the first and second promoters, wherein the at least one enhancer element is dependent on a transcriptional transregulator, wherein preferably introduction of the transcriptional transregulator into the cell induces transcription from the first and second promoters.

The insect cell can be any cell that is suitable for the production of heterologous proteins. Preferably the insect cell allows for replication of baculoviral vectors and can be maintained in culture. More preferably the insect cell also allows for replication of recombinant parvoviral vectors, including rAAV vectors. For example, the cell line used can be from Spodoptera frugiperda, Drosophila cell lines, or mosquito cell lines, e.g., Aedes albopictus derived cell lines. Preferred insect cells or cell lines are cells from the insect species which are susceptible to baculovirus infection, including e.g. S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). A preferred insect cell according to the invention is an insect cell for production of recombinant parvoviral vectors.

One of ordinary skill in the art knows how to stably introduce a nucleotide sequence into the insect genome and how to identify a cell having such a nucleotide sequence in the genome. The incorporation into the genome may be aided by, for example, the use of a vector comprising nucleotide sequences highly homologous to regions of the insect genome. The use of specific sequences, such as transposons, is another way to introduce a nucleotide sequence into a genome. The incorporation into the genome may be through one or more than one steps. Reference to the term “integrated” will be known to one in the art to also mean “stably integrated”.

Growing conditions for insect cells in culture, and production of heterologous products in insect cells in culture are well-known in the art and described e.g. in the above cited references on molecular engineering of insect cells (see also WO2007/046703).

An “insect cell-compatible vector” or “vector” is understood to be a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In a preferred embodiment, the vector is a baculovirus, a viral vector, or a plasmid. In a more preferred embodiment, the vector is a baculovirus, i.e. the nucleic acid construct is a baculovirus-expression vector. Baculovirus-expression vectors and methods for their use are described for example in: Summers and Smith, 1986, “A Manual of Methods for Baculovirus Vectors and Insect Culture Procedures”, Texas Agricultural Experimental Station Bull. No. 7555, College Station, Tex.; Luckow, 1991, In Prokop et al., “Cloning and Expression of Heterologous Genes in Insect Cells with Baculovirus Vectors' Recombinant DNA Technology and Applications”, 97-152; King and Possee, 1992, “The baculovirus expression system”, Chapman and Hall, United Kingdom; O'Reilly, Miller, and Luckow, 1992, “Baculovirus Expression Vectors: A Laboratory Manual”, New York; Freeman and Richardson, 1995, “Baculovirus Expression Protocols”, Methods in Molecular Biology, volume 39; U.S. Pat. No. 4,745,051; US2003148506; and WO 03/074714.

The number of nucleic acid constructs employed in the insect cell for the production of the recombinant parvoviral (rAAV) vector is not limiting in the invention. For example, one, two, three or more separate constructs can be employed to produce rAAV in insect cells in accordance with the methods of the present invention. If two constructs are employed, one construct may comprise the nucleotide sequence comprising the transgene that is flanked by at least one parvoviral ITR sequence and the other construct may then comprise expression cassettes for respectively the Rep and Cap proteins. If three constructs are employed, one construct may comprise the nucleotide sequence comprising the transgene that is flanked by at least one parvoviral ITR sequence, another construct may comprise an expression cassette for the Cap proteins and still another construct may comprise one or more expression cassettes for the Rep protein, e.g. two expression cassettes, one for each of the Rep 78 and 52 proteins, optionally either codon optimized, AT-optimized or GC-optimized, in order to minimize or prevent recombination, as described hereinafter. It is hereby understood that at least some of the nucleic acid constructs, preferably those comprising the one or more expression cassettes for the Rep proteins, can be stably integrated into the genome of the insect cell.

The inventors of the current invention have further optimised the design of an inducible insect cell expression vector (e.g. for expression of the Rep proteins, such as iRep) in two ways. Firstly, by investigating the use of alternative baculovirus promoters in regulating AAV gene expression. So far, the polyhedron promoter (polH) has been the most extensively studied promoter in AAV production, in the BEV setting (van Oers, M. M., et al., 2015). Although alternative late promoters, such as p10, have been reported to share a host factor with polH (Ghosh, S., et al., 1998), other baculovirus promoters have been reported to exhibit different induction intensities and temporal profiles (Dong, Z. Q. et al., 2018; Lin, C. H & Jarvis, D. L., 2013; Martinez-Solis, M., et al., 2016). Nevertheless, their potential use for AAV production in insect cells has never been reported thus far. Secondly, tighter regulation on the inducible expression is also explored in this study. This is e.g. desirable for inducible expression of AAV Rep, as Rep proteins can be toxic for the host cells and control of expression is thus required. The use of baculovirus homologous region (hr) 2 or hr2.09 enhancer sequence in combination with polH has become the default molecular design for the inducible OneBac platform (Aslanidi, G., et al. supra). Here, the inventors examined the potential use of alternative baculovirus promoters in combination with other baculovirus hr enhancer sequences. By studying the different baculovirus promoters and enhancers, also in different molecular conformations, the inventors have optimised expression of AAV genes (Cap, Rep) to obtain a stable and robust AAV production platform yielding high quality AAV batches with high titer.

This approach includes the adoption of alternative and non-conservative baculovirus promoters (p10, 39k, p6.9, pSel120) with similar or distinct expression intensities and temporal profiles which advantageously creates an inducible expression construct regulating wild-type (wt) single- or split-cassette AAV Rep, or other AAV gene expression. This then will enable the production of an inducible plasmid vector construct that is less prone to cis:trans promoter competition upon recombinant baculovirus transactivation. In addition, it will enable the use of less/non-leaky baculovirus hr enhancers to enable tighter regulation of the inducible plasmid vector construct.

Additional benefits of the invention include an improved AAV production yield and quality over the OneBac and insect cell platform; the provision of an inducible promoter that is truly silent when not induced, thereby allowing to avoid expression of toxic AAV genes, such as Rep, when switched ‘off’, which allows more viable and stable AAV packaging cells; and the adaptation of split-cassette Rep AAV design into inducible plasmid vectors.

### Promoters

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells. A “cryptic promoter” is an epigenetically silenced promoter which may be activated.

In a preferred embodiment, the ratio of expression of the Rep78 versus the Rep52 protein is regulated by one or more of the following: (a) the second promoter is stronger than the first promoter, as e.g. determined by reporter gene expression (e.g. luciferase or SEAP), or northern or western blot; (b) the presence of nucleotide spacer or more and/or stronger enhancer elements at upstream of the second expression cassette as compared to the first expression cassette; (c) the nucleotide sequence coding for the parvoviral Rep52 protein has a higher codon adaptation index as compared to the nucleotide sequence coding for the Rep78 protein; (d) temperature optimization of the parvoviral Rep protein; and (e) variant Rep proteins with one or more alterations in the amino acid sequence as compared to a corresponding wild-type Rep protein and wherein the one or more amino acid alteration result in increases in the activity of the Rep function as assessed by detecting increased AAV production in insect cells. Methods for generation, selection and/or screening of variant Rep proteins with increased activity of Rep function as assessed by detecting increased AAV production in insect cells may be obtained by adaptation to insect cells of the methods described in US20030134351 for obtaining variant Rep proteins with increased function with respect to AAV production in mammalian cells. Variant Rep proteins with one or more alterations in the amino acid sequence as compared to a corresponding wild-type Rep protein are herein understood to include Rep proteins with one or more amino acid substitutions, insertions and/or deletions in the variant amino acid sequence as compared to the amino acid sequence of a corresponding wild type Rep protein.

The second promoter being stronger than the first promoter means that more mRNA molecules coding for a Rep52 protein are expressed than mRNA molecules coding for a Rep78 protein. An equally strong promoter may be used, since expression of Rep52 protein will then be increased as compared to expression of Rep78 protein. The strength of the promoter may be determined by the expression that is obtained under conditions that are used in the method of the invention.

In one embodiment, the first and second promoters are baculoviral promoters. In one embodiment, the first and second promoters are distinct. In one embodiment, the first promoter is a delayed early baculoviral promoter, such as the 39k promoter. In one embodiment, the second promoter is a late or very late baculovirus promoter, such as the polH, p10, p6.9 and pSel120 promoters. Consequently, in one embodiment, the first promoter is a delayed early baculoviral promoter and the second promoter is a late or very late baculovirus promoter. Therefore, in one embodiment, the first promoter is the 39k promoter and the second promoter is selected from the group consisting of the p10, p6.9 and pSel120 promoters.

In one embodiment, the first and second promoters are integrated in the cell's genome in opposite directions of transcription.

As described below, for production of a complete parvoviral gene therapy vector virion, the cell preferably further comprises an expression cassette comprising a nucleotide sequence comprising parvoviral capsid protein coding sequences operably linked to a third promoter for expression in the insect cell. In one embodiment, the first, second and third promoters are baculoviral promoters. In one embodiment, the first, second and third promoters are distinct. In one embodiment, the first promoter is a delayed early baculoviral promoter, such as the 39k promoter. In one embodiment, the second promoter is a late or very late baculovirus promoter, such as the polH, p10, p6.9 and pSel120 promoters. Consequently, in one embodiment, the first promoter is a delayed early baculoviral promoter and the second promoter is a late or very late baculovirus promoter. Therefore, in one embodiment, the first promoter is the 39k promoter and the second promoter is selected from the group consisting of the polH, p10, p6.9 and pSel120 promoters. In one embodiment, the first promoter is active before the third promoter.

### Enhancer

An “enhancer element” or “enhancer” is meant to define a sequence which enhances the activity of a promoter (i.e. increases the rate of transcription of a sequence downstream of the promoter) which, as opposed to a promoter, does not possess promoter activity, and which can usually function irrespective of its location with respect to the promoter (i.e. upstream, or downstream of the promoter). Enhancer elements are well-known in the art. Non-limiting examples of enhancer elements (or parts thereof) which could be used in the present invention include baculovirus enhancers and enhancer elements found in insect cells. It is preferred that the enhancer element increases in a cell the mRNA expression of a gene, to which the promoter it is operably linked, by at least 25%, more preferably at least 50%, even more preferably at least 100%, and most preferably at least 200% as compared to the mRNA expression of the gene in the absence of the enhancer element. mRNA expression may be determined for example by quantitative RT-PCR.

Herein it is preferred to use an enhancer element to enhance the expression of parvoviral Rep proteins. In a preferred embodiment at least one enhancer element that is operably linked to the (first and/or second) promoter as defined herein that is operably linked to a nucleotide sequence encoding an mRNA, translation of which in the cell produces a parvoviral Rep protein, is an enhancer element that is dependent on a transcriptional transregulator. A transcriptional transregulator-dependent enhancer element is herein understood as an enhancer element that activates transcription of a promoter operably linked thereto when bound by a transcriptional transregulator protein provided in trans.

Thus, in a further preferred embodiment, the transcriptional transregulator-dependent enhancer element comprises at least one baculovirus enhancer element and/or at least one ecdysone responsive element. Preferably the transcriptional transregulator is a baculoviral immediate-early protein (IE1) or its spice variant (1E0) and the transcriptional transregulator-dependent enhancer element is a baculoviral homologous region (hr) enhancer element, wherein preferably the baculovirus is Autographa californica multicapsid nucleopolyhedronvirus. IE1 is a highly conserved, 67-kDa DNA binding protein that transactivates baculovirus early gene promoters and supports late gene expression in plasmid transfection assays (see e.g. Olson et al., 2002, J Virol., 76:9505-9515). AcMNPV IE1 possesses separable domains that contribute to promoter transactivation and DNA binding. The N-terminal half of this 582-residue phosphoprotein contains transcriptional stimulatory domains from residue 8 to 118 and 168 to 222. IE1 binds to the —28-bp imperfect palindrome (28-mer) that constitutes repetitive sequences within multiple homologous regions (hrs) found dispersed throughout the AcMNPV genome. The hr 28-mer is the minimal sequence motif required for IE1-mediated enhancer and origin-specific replication functions.

In one embodiment, the hr enhancer element is an hr enhancer element other than hr2-0.9 US 2012/100606 Al). In a further embodiment, the hr enhancer element is selected from the group consisting of hrl, hr3, hr4b and hr5, of which hr4b and hr5 are preferred, of which hr4b is most preferred. In an alternative embodiment, the hr enhancer element is a variant hr enhancer element, such as e.g. a non-naturally occurring designed element. The variant hr enhancer element preferably comprises at least one copy of the hr 28-mer sequence CTTTACGAGTAGAATTCTACGCGTAAAA (SEQ ID NO. 32) and/or at least one copy of a of a sequence of which at least 18, 20, 21, 22, 23, 24, 25, 26, or 27 nucleotides are identical to sequence CTTTACGAGTAGAATTCTACGCGTAAAA (SEQ ID NO. 32) and which preferably binds to the baculoviral IE1 protein, more preferably to the AcMNPV IE1 protein. The variant hr enhancer element is further preferably functionally defined in that when the variant element is operably linked to an expression cassette comprising a reporter gene operably linked to the polH promoter, a) under non-inducing conditions, the cassette with the variant element produces less reporter transcript than an otherwise identical expression cassette which comprises the hr2-0.9 element instead of the variant element, or the cassette with the variant element produces less than a factor 1.1, 1.2, 1.5, 2, 5 or 10 of the amount reporter transcript produced by an otherwise identical expression cassette which comprises the hr4b element instead of the variant element; and b) under inducing conditions, the cassette with the variant element produces at least 50, 60, 70, 80, 90 or 100% of the amount of reporter transcript produced by an otherwise identical expression cassette which comprises the hr4b or the hr2-0.9 element instead of the variant element. Non-inducing conditions are understood as condition in which there is no IE1 protein present in the cell wherein the cassettes are tested and inducing conditions are understood to be conditions wherein sufficient IE1 protein is present to obtain maximal reporter expression with the reference cassettes comprising the hr4b or the hr2-0.9 element. Binding of the variant hr enhancer element to the baculoviral IE1 protein can be assayed by using a mobility shift assay as e.g. described by Rodems and Friesen (J Virol. 1995; 69(9):5368-75).

In one embodiment, the at least one enhancer element is present in between the first and second promoters. Consequently, in one embodiment, the first and second promoters are integrated in the cell's genome in opposite directions of transcription and the at least one enhancer element is present in between the first and second promoters. In a further embodiment, two enhancer elements are present in between the first and second promoters. When using Bac polH Cap Trans for induction, a relatively weaker transactivation profile is observed due to i) the cis:trans promoter competition between the two polH promoters used (for Cap in the Bac polH Cap Trans and for Rep in the expression plasmid) and ii) the adoption of non-leaky but relatively weaker hr such as hr4b. In one embodiment, there is the use of such a non-leaky expression platform, for example, the use of a relatively weaker hr such as hr4b. In a further embodiment, there is compatibility with the use of Bac polH Cap Trans. In still a further embodiment, the hr4b enhancer is combined with the p10 promoter. Such a combination may be made to regulate single-cassette AAV2 Rep with a strong wild-type ATG start codon.

### Replicase (Rep) Proteins

Parvoviral, especially AAV, replicases are non-structural proteins encoded by the rep gene. In wild type parvoviruses the rep gene produces two overlapping messenger ribonucleic acids (mRNA) with different length, due to an internal P19 promoter. Each of these mRNA can be spliced out or not to eventually generate four Rep proteins, Rep78, Rep68, Rep52 and Rep40. The Rep78/68 and Rep52/40 are important for the ITR-dependent AAV genome or transgene replication and viral particle assembly. Rep78/68 serve as a viral replication initiator proteins and act as replicase for the viral genome (Chejanovsky and Carter, J Virol., 1990, 64:1764-1770; Hong et al., Proc Natl Acad Sci USA, 1992, 89:4673-4677; Ni., et al., J Virol., 1994, 68:1128-1138). The Rep52/40 protein is DNA helicase with 3′ to 5′ polarity and plays a critical role during packaging of viral DNA into empty capsids, where they are thought to be part of the packaging motor complex (Smith and Kotin, J. Virol., 1998, 4874 — 4881; King, et al., EMBO J., 2001, 20:3282-3291). To produce AAV from the baculoviral vectors in an insect cell platform, the presence of both Rep68 and Rep40 is not prerequisite (Urabe, et al., 2002).

A nucleotide sequence encoding a parvoviral Rep protein, is herein understood as a nucleotide sequence encoding at least one of the two non-structural Rep proteins, Rep 78 and Rep52, that together are required and sufficient for parvoviral vector production in insect cells. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 8 and 9) or primates (e.g., serotypes 1 and 4). An example of a nucleotide sequence encoding parvovirus Rep proteins is given in SEQ ID NO. 37 (see SEQ ID NO. 5 of WO 2009/104964, included herein by reference), which depicts a part of the AAV serotype-2 sequence genome encoding the Rep proteins. The Rep78 coding sequence comprises nucleotides 11-1876 and the Rep52 coding sequence comprises nucleotides 683 -1876, also depicted separately in SEQ ID NO.s 37 and 39 (see SEQ ID NO.s 5 and 7 of WO 2009/104964, included herein by reference). It is understood that the exact molecular weights of the Rep78 and Rep52 proteins, as well as the exact positions of the translation initiation codons may differ between different parvoviruses. However, the skilled person will know how to identify the corresponding position in nucleotide sequence from other parvoviruses than AAV-2.

According to the invention, the cell preferably comprises integrated into its genome a first nucleic acid construct that comprises at least a first and a second expression cassette for expression of the parvoviral Rep proteins.

The first expression cassette comprises a first promoter operably linked to a nucleotide sequence encoding an mRNA, translation of which in the cell produces at least one of parvoviral Rep 78 and 68 proteins. A nucleotide sequence encoding an mRNA, translation of which in an insect cell produces at least one of parvoviral Rep 78 and 68 proteins can be defined as a nucleotide sequence: a) that encodes a polypeptide comprising an amino acid sequence that has at least 50, 60, 70, 80, 88, 89, 90, 95, 97, 98, or 99% sequence identity with the amino acid sequence of SEQ ID NO. 40 (see SEQ ID NO. 8 of WO 2009/104964, included herein by reference); b) that has at least 50, 60, 70, 80, 81, 82, 85, 90, 95, 97, 98, or 99% sequence identity with the nucleotide sequence of positions 11-1876 of SEQ ID NO. 39 (see SEQ ID NO. 7 of WO 2009/104964, included herein by reference); c) the complementary strand of which hybridises to a nucleic acid molecule sequence of (a) or (b); and d) nucleotide sequences the sequence of which differs from the sequence of a nucleic acid molecule of (c) due to the degeneracy of the genetic code. Preferably, the nucleotide sequence encodes an mRNA, translation of which in an insect cell produces only at least one of parvoviral Rep 78 and 68 proteins. It is understood that translation the mRNA in an insect cell will usually only produce at least a parvoviral Rep 78 protein and need not produce a parvoviral Rep 68 protein. It is further understood that while the nucleotide sequence encodes an mRNA, translation of which in an insect cell produces only at least one of parvoviral Rep 78 and 68 proteins (and not the parvoviral Rep 52 and 40 proteins), this does not exclude that the nucleotide sequence comprises an internal endogenous parvoviral P19 promoter that is active in insect cells and which produces an additional mRNA, translation of which in an insect cell produces at least one of parvoviral Rep 52 and 40 proteins. In a preferred embodiment, the nucleotide sequence that encodes the mRNA, translation of which in an insect cell produces only at least one of parvoviral Rep 78 and 68 proteins, indeed comprises a parvoviral P19 promoter, which preferably is intact or at least active in an insect cell.

The second expression cassette comprises a second promoter operably linked to a nucleotide sequence encoding an mRNA, translation of which in the cell produces at least one of parvoviral Rep 52 and 40 proteins. A nucleotide sequence encoding an mRNA, translation of which in an insect cell produces at least one of parvoviral Rep 52 and 40 proteins can be defined as a nucleotide sequence: a) that encodes a polypeptide comprising an amino acid sequence that has at least 50, 60, 70, 80, 88, 89, 90, 95, 97, 98, or 99% sequence identity with the amino acid sequence of SEQ ID NO. 38 (see SEQ ID NO. 6 of WO 2009/104964, included herein by reference); b) that has at least 50, 60, 70, 80, 81, 82, 85, 90, 95, 97, 98, or 99% sequence identity with the nucleotide sequence of any one of SEQ ID NO.s 33-37 (see SEQ ID NO.s 1-5 of WO 2009/104964, included herein by reference) and SEQ ID NO. 15, of which SEQ ID NO. 15 is preferred; c) the complementary strand of which hybridises to a nucleic acid molecule sequence of (a) or (b); and, d) nucleotide sequences the sequence of which differs from the sequence of a nucleic acid molecule of (c) due to the degeneracy of the genetic code. Preferably, the nucleotide sequence encodes an mRNA, translation of which in an insect cell produces only at least one of parvoviral Rep 52 and 40 proteins. Thereby it is understood that the nucleotide sequence encoding the parvoviral Rep 52 and/or 40 proteins is not part of a larger coding sequence that also encodes the parvoviral Rep 78 and/or 68 proteins. Preferably the nucleotide sequence encoding an mRNA, translation of which in the cell produces only at least one of parvoviral Rep 52 and 40 proteins comprises an open reading frame that consists of the amino acid sequence from the translation initiation codon to the most C-terminal amino acid of the at least one of parvoviral Rep 52 and 40 proteins, more preferably, the open reading frame is the only open reading frame comprised in the nucleotide sequence encoding an mRNA. It is further understood that translation the mRNA in an insect cell will usually only produce at least a parvoviral Rep 52 protein and need not produce a parvoviral Rep 40 protein.

Preferably, the nucleotide sequence encodes parvovirus Rep proteins that are functionally active in the sense that they have the required activities of viral replication initiator protein, replicase of the viral genome, DNA helicase and packaging of viral DNA into empty capsids as described above, sufficient for parvoviral vector production in insect cells.

In one embodiment, possible false translation initiation sites in the Rep protein coding sequences, other than the Rep78 and Rep52 translation initiation sites are eliminated. In one embodiment, putative splice sites that may be recognised in insect cells are eliminated from the Rep protein coding sequences. Elimination of these sites will be well understood by an artisan of skill in the art.

In a preferred embodiment, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90, 91, 92, 93, 94, 95, 96, 97, 98, 99 or 100% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90, 89, 88, 87, 86, 85, 84, 83, 82, 81, 80, 79, 78, 77, 76, 75, 74, 73, 72, 71, 70, 69, 68, 67, 66, 60% identical.

In a further embodiment, the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40. Preferably, however, the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins.,

The adaptiveness of a nucleotide sequence encoding the common amino acid sequence to the codon usage of the host cell can be expressed as codon adaptation index (CAI). Preferably the codon usage is adapted to the insect cell wherein Rep proteins with the common amino acid sequence are expressed. Usually this will be a cell of the genus Spodoptera, more preferably a Spodoptera frugiperda cell. The codon usage is thus preferably adapted to Spodoptera frugiperda or to an Autographa californica nucleopolyhedrovirus (AcMNPV) infected cell. A codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (Sharp and Li, 1987, Nucleic Acids Research 15: 1281-1295; also see: Kim et al., Gene. 1997, 25 199:293-301; zur Megede et al., Journal of Virology, 2000, 74: 2628-2635).

Preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.1, 0.2, 0.3, 0.4, 0.5, 0.6, 0.7 or 0.8, whereby more preferably, the CAI of the nucleotide sequence coding for the common amino acid sequence in the at least one of parvoviral Rep 52 and 40 proteins is at least 0.5, 0.6, 0.7, 0.8, 0.9 or 1.0.

Therefore, in an alternative embodiment, the at least one of parvoviral Rep 78 and 68 proteins and the at least one of parvoviral Rep 52 and 40 proteins comprise a common amino acid sequence comprising the amino acid sequence from the second amino acid to the most C-terminal amino acid of the at least one of parvoviral Rep 52 and 40 proteins, wherein the common amino acid sequences of the at least one of parvoviral Rep 78 and 68 proteins and the at least one of parvoviral Rep 52 and 40 proteins are at least 90% identical, and wherein the nucleotide sequence encoding the common amino acid sequence of the at least one of parvoviral Rep 78 and 68 proteins and the nucleotide sequence encoding the common amino acid sequences of the at least one of parvoviral Rep 52 and 40 proteins are less than 90% identical, and the nucleotide sequence encoding the common amino acid sequence of the at least one of parvoviral Rep 78 and 68 proteins has an improved codon usage bias for the cell as compared to the nucleotide sequence encoding the common amino acid sequences of the at least one of parvoviral Rep 52 and 40, or wherein the nucleotide sequence encoding the common amino acid sequence of the at least one of parvoviral Rep 52 and 40 proteins has an improved codon usage bias for the cell as compared to the nucleotide sequence encoding the common amino acid sequences of the at least one of parvoviral Rep 78 and 68 proteins, wherein preferably, the difference in codon adaptation index between the nucleotide sequences coding for the common amino acid sequences in the at least one of parvoviral Rep 78 and 68 proteins and the at least one of parvoviral Rep 52 and 40 proteins is at least 0.2. In a preferred embodiment the nucleotide sequence encoding the common amino acid sequence of the at least one of parvoviral Rep 52 and 40 proteins with an improved codon usage bias for the insect cell has a nucleotide sequence of which at least 80, 85, 90, 95, 96, 97, 98, 99 or 100% of the codons are identical to the codons in SEQ ID NO. 15.

Temperature optimization of the parvoviral Rep protein refers to use the optimal condition with respect to both the temperature at which the insect cell will grow and Rep is functioning. A Rep protein may for example be optimally active at 37° C., whereas an insect cell may grow optimally at 28° C. A temperature at which both the Rep protein is active and the insect cell grows may be 30° C. In a preferred embodiment, the optimized temperature is more than 27, 28, 29, 30, 31, 32, 33, 34 or 35° C. and/or less than 37, 36, 35, 34, 33, 32, 31, 30 or 29° C.

In one embodiment, the first and second expression cassettes in the cell are optimised to obtain a desired molar ratio of Rep78 to Rep52 in the (insect) cell. Preferably, the combination of first and second expression cassettes in the cell produces a molar ratio of Rep78 to Rep52 in the range of 1:10 to 10:1, 1:5 to 5:1, or 1:3 to 3:1 in the (insect) cell. More preferably, the first nucleic acid construct produces a molar ratio of Rep78 to Rep52 that is at least 1:2, 1:3, 1:5 or 1:10. The molar ration of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

A desired molar ratio of Rep78 to Rep52 can be obtained by the choice of the promoters in respectively the first and second expression cassettes as herein further described above. Alternatively or in combination, the desired molar ratio of Rep78 to Rep52 can be obtained by using means to reduce the steady state level of the at least one of parvoviral Rep 78 and 68 proteins.

Thus, in one embodiment, the nucleotide sequence encoding the mRNA for the at least one of parvoviral Rep 78 and 68 proteins comprises a modification that affects a reduced steady state level of the at least one of parvoviral Rep 78 and 68 proteins. The reduced steady state condition can be achieved for example by truncating the regulation element or upstream promoter (Urabe et al., supra, Dong et al., supra), adding protein degradation signal peptide, such as the PEST or ubiquitination peptide sequence, substituting the start codon into a more suboptimal one, or by introduction of an artificial intron as described in WO 2008/024998.

In a preferred embodiment, the nucleotide sequence encoding at least one of parvoviral Rep78 and 68 proteins comprises an open reading frame that starts with a suboptimal translation initiation codon. The suboptimal initiation codon preferably is an initiation codon that affects partial exon skipping. Partial exon skipping is herein understood to mean that at least part of the ribosomes do not initiate translation at the suboptimal initiation codon of the Rep78 protein but may initiate at an initiation codon further downstream, whereby preferably the (first) initiation codon further downstream is the initiation codon of the Rep52 protein. Alternatively, the nucleotide sequence encoding at least one of parvoviral Rep78 and 68 proteins comprises an open reading frame that starts with a suboptimal translation initiation codon and has no initiation codons further downstream. The suboptimal initiation codon preferably affects partial exon skipping upon expression of the nucleotide sequence in an insect cell. Preferably, the suboptimal initiation codon affects partial exon skipping in an insect cell so as to produce in the insect cell a molar ratio of Rep78 to Rep52 in the range of 1:10 to 10:1, 1:5 to 5:1, or 1:3 to 3:1. The molar ratio of the Rep78 and Rep52 may be determined by means of Western blotting, preferably using a monoclonal antibody that recognizes a common epitope of both Rep78 and Rep52, or using e.g. a mouse anti-Rep antibody (303.9, Progen, Germany; dilution 1:50).

The term “suboptimal initiation codon” herein not only refers to the tri-nucleotide initiation codon itself but also to its context. Thus, a suboptimal initiation codon may consist of an “optimal” ATG codon in a suboptimal context, e.g. a non-Kozak context. However, more preferred are suboptimal initiation codons wherein the tri-nucleotide initiation codon itself is suboptimal, i.e. is not ATG. Suboptimal is herein understood to mean that the codon is less efficient in the initiation of translation in an otherwise identical context as compared to the normal ATG codon. Preferably, the efficiency of suboptimal codon is less than 90, 80, 60, 40 or 20% of the efficiency of the normal ATG codon in an otherwise identical context. Methods for comparing the relative efficiency of initiation of translation are known per se to the skilled person. Preferred suboptimal initiation codons may be selected from ACG, TTG, CTG, and GTG. More preferred is ACG. A nucleotide sequence encoding parvovirus Rep proteins, is herein understood as a nucleotide sequence encoding the non-structural Rep proteins that are required and sufficient for parvoviral vector production in insect cells such the Rep78 and Rep52 proteins.

### Capsid Proteins

For production of a complete parvoviral gene therapy vector virion, the cell preferably further comprises a further (third) expression cassette comprising a nucleotide sequence comprising parvoviral capsid protein coding sequences operably linked to a third promoter for expression in the insect cell.

A nucleotide sequence encoding a parvoviral capsid (Cap) protein is herein understood to comprise nucleotide sequences encoding one or more of the three parvoviral capsid proteins, VP1, −2 and −3. The parvovirus nucleotide sequence preferably is from a dependovirus, more preferably from a human or simian adeno-associated virus (AAV) and most preferably from an AAV which normally infects humans (e.g., serotypes 1, 2, 3A, 3B, 4, 5, 6, 7, 8, 9, 10, 11, 12 or 13) or primates (e.g., serotypes 1 and 4), of which the nucleotide and amino acid sequences are listed in Lubelski et al. US2017356008, which is incorporated herein in its entirety by reference. Hence, the nucleic acid construct according to the present invention can comprise an entire open reading frame for AAV capsid proteins as disclosed by Lubelski et al. US2017356008. Alternatively, the sequence can be man-made, for example, the sequence may be a hybrid form or may be codon optimized, such as for example by codon usage of AcmNPv or Spodoptera frugiperda. For example, the capsid sequence may be composed of the VP2 and VP3 sequences of AAV1 whereas the remainder of the VP1 sequence is of AAV5. A preferred capsid protein is AAV5 or an AAV2/5 hybrid, preferably (SEQ ID NO. 30 and 29, respectively in this application) or AAV8, preferably SEQ ID NO. 41 (see SEQ ID NO. 28 in Lubelski et al. US2017356008). Thus, in a preferred embodiment, the AAV capsid proteins are AAV serotype 5, hybrid serotype 2/5 or AAV serotype 8 capsid proteins that have been modified according to the invention. More preferably, the AAV capsid proteins are AAV serotype 5 capsid proteins that have been modified according to the invention. More preferably, the cap-coding sequences are at least CAP AAV2/5 (SEQ ID NO. 29) and AAV5 (SEQ ID NO. 30). It is understood that the exact molecular weights of the capsid proteins, as well as the exact positions of the translation initiation codons may differ between different parvoviruses. However, the skilled person will know how to identify the corresponding position in nucleotide sequence from other parvoviruses than AAV5. Alternatively, the sequence encoding AAV capsid proteins is a man-made sequence, for example as a result of directed evolution experiments. This can include generation of capsid libraries via DNA shuffling, error prone PCR, bioinformatics rational design, site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, Calif., USA; Sep. 1-5, 2004, pages 3520-3523; Asuri et al., 2012, Molecular Therapy (2):329-3389; Lisowski et al., 2014, Nature 506(7488):382-386, herein incorporated by reference.

In a preferred embodiment of the invention, the open reading frame encoding a VP1 capsid protein starts with non-canonical translation initiation codon selected from the group consisting of: ACG, ATT, ATA, AGA, AGG, AAA, CTG, CTT, CTC, CTA, CGA, CGC, TTG, TAG and GTG. Preferably, the non-canonical translation initiation codon is selected from the group consisting of GTG, CTG, ACG, and TTG, more preferably the non-canonical translation initiation codon is CTG.

The nucleotide sequence of the invention for expression of the AAV capsid proteins further preferably comprises at least one modification of the nucleotide sequence encoding AAV VP1 capsid protein selected from among a G at nucleotide position 12, an A at nucleotide position 21, and a C at nucleotide position 24 of the VP1 open reading frame, wherein the nucleotide positions correspond to the nucleotide positions of the wild-type nucleotide sequences. A “potential/possible false start site” or “potential/possible false translation initiation codon” is herein understood to mean an in-frame ATG codon located in the coding sequence of the capsid protein(s). Elimination of possible false start sites for translation within the VP1 coding sequences of other serotypes will be well understood by an artisan of skill in the art, as will be the elimination of putative splice sites that may be recognized in insect cells. For example, the modification of the nucleotide at position 12 is not required for recombinant AAV5, since the nucleotide T is not giving rise to a false ATG codon. Specific examples of a nucleotide sequence encoding parvovirus capsid proteins are given in SEQ ID NO. 44, 45 and 46. Nucleotide sequences encoding parvoviral Cap and/or Rep proteins of the invention may also be defined by their capability to hybridise with the nucleotide sequences of SEQ ID NO. 44, 45, 46 and 33 to 37, respectively, under moderate, or preferably under stringent hybridisation conditions.

The capsid protein coding sequences may be present in various forms. E.g. separate coding sequences for each of the capsid proteins VP1, -2 and -3 may be used, whereby each coding sequence is operably linked to expression control sequences for expression in an insect cell. More preferably, however, the third expression cassette comprises a nucleotide sequence comprising a single open reading frame encoding all three of the parvoviral (AAV) VP1, VP2, and VP3 capsid proteins, wherein the initiation codon for translation of the VP1 capsid protein is a suboptimal initiation codon that is not ATG as e.g. described by Urabe et al., (2002, supra) and in WO2007/046703. A suboptimal initiation codon for the VP1 capsid protein may be as defined above for the Rep78 protein. More preferred suboptimal initiation codons for the VP1 capsid protein may be selected from ACG, TTG, CTG and GTG, of which CTG and GTG are most preferred. In an alternative embodiment, the second expression cassette comprises a nucleotide sequence comprising a single open reading frame encoding all three of the parvoviral (AAV) VP1, VP2, and VP3 capsid proteins, wherein the initiation codon for translation of the VP1 capsid protein is ATG and wherein the mRNA coding for the VP1 capsid protein as encoded in the nucleotide sequence comprises an alternative start codon which is out of frame with the open reading frame the VP1 capsid protein (as described in WO2019/016349). Preferably, the alternative start codon is selected from the group consisting of CTG, ATG, ACG, TTG, GTG, CTC and CTT, of which ATG is preferred. Preferably, the AAV capsid proteins are AAV5 serotype capsid proteins. Preferably in this embodiment, the nucleotide sequence comprises an alternative open reading frame starting with the alternative start codon that encompasses said ATG translation initiation codon for VP1, whereby preferably, the alternative open reading frame following the alternative start codon encodes a peptide of up to 20 amino acids.

The nucleotide sequence comprised in the second expression cassette for expression of the capsid proteins may further comprise one or more modifications as described in WO2007/046703. Various further modifications of VP coding regions are known to the skilled artisan which could either increase yield of VP and virion or have other desired effects, such as altered tropism or reduce antigenicity of the virion. These modifications are within the scope of the present invention.

In one embodiment, the expression of VP1 is increased as compared to the expression of VP2 and VP3. VP1 expression may be increased by supplementation of VP1, by introduction into the insect cell of a single vector comprising nucleotide sequences for the VP1 as has been described in WO 2007/084773.

Typically, in a method of the invention, at least one open reading frame comprising nucleotide sequences encoding the VP1, VP2 and VP3 capsid proteins or at least one open reading frame, comprising an open reading frame comprising nucleotide sequences encoding at least one of the Rep78 and Rep68 proteins. In one embodiment, the VP1, VP2 and VP3 capsid proteins or at least one open reading frame comprising an open reading frame comprising nucleotide sequences encoding at least one of the Rep78 and Rep68 proteins does not comprise an artificial intron (or a sequence derived from an artificial intron). That is to say, at least open reading frames used to encode Rep or VP proteins will not comprise an artificial intron. By artificial intron is meant to be an intron which would not naturally occur in an adeno-associated virus Rep or Cap sequence, for example an intron which has been engineered so as to permit functional splicing within an insect cell. An artificial intron in this context therefore encompass wild-type insect cell introns. An expression cassette of the invention may comprise native truncated intron sequence (by native is meant sequence naturally occurring in an adeno-associated virus)—such sequences are not intended to fall within the meaning of artificial intron as defined herein. In the invention, one possibility is that no open reading frame comprising nucleotide sequences encoding the VP1, VP2 and VP3 capsid proteins and/or no open reading frame comprising nucleotide sequences encoding at least one of the Rep78 and Rep68 proteins comprises an artificial intron.

Preferably the nucleotide sequence of the invention encoding the AAV capsid proteins is operably linked to expression control sequences for expression in an insect cell. These expression control sequences will at least include a promoter that is active in insect cells. A suitable promoter for transcription of the nucleotide sequence of the invention encoding of the AAV capsid proteins is e.g. the polyhedron promoter (polH), such a polH promoter SEQ ID NO, 42 and shortened version thereof SEQ ID NO. 43 (see SEQ ID NO.:53 and shortened version thereof SEQ ID NO. 54 in Lubelski et al. US2017356008). However, other promoters that are active in insect cells and that may be selected according to the invention are known in the art, e.g. a polyhedrin (polH) promoter, p10 promoter, p35 promoter, 4×Hsp27 EcRE+minimal Hsp70 promoter, deltaEl promoter, El promoter or 1E-1 promoter and further promoters described in the above references.

### Viral Vectors

The present invention relates to the use of parvoviruses, in particular dependoviruses such as infectious human or simian AAV, and the components thereof (e.g., a parvovirus genome) for use as vectors for introduction and/or expression of nucleic acids in mammalian cells, preferably human cells. In particular, the invention relates to improvements in productivity of such parvoviral vectors when produced in insect cells.

A “parvoviral vector” is defined as a recombinantly produced parvovirus or parvoviral particle that comprises a polynucleotide to be delivered into a host cell, either in vivo, ex vivo or in vitro. Examples of parvoviral vectors include e.g., adeno-associated virus vectors. Herein, a parvoviral vector construct refers to the polynucleotide comprising the viral genome or part thereof, and a transgene. Viruses of the Parvoviridae family are small DNA viruses. The family Parvoviridae may be divided between two subfamilies: the Parvovirinae, which infect vertebrates, and the Densovirinae, which infect invertebrates, including insects. Members of the subfamily Parvovirinae are herein referred to as the parvoviruses and include the genus Dependovirus. As may be deduced from the name of their genus, members of the Dependovirus are unique in that they usually require coinfection with a helper virus such as adenovirus or herpes virus for productive infection in cell culture. The genus Dependovirus includes AAV, which normally infects humans (e.g., serotypes 1, 2, 3A, 3B, 4, 5, 6, 7, 8, 9, 10, 11, 12 and 13) or primates (e.g., serotypes 1 and 4), and related viruses that infect other warm-blooded animals (e.g., bovine, canine, equine, and ovine adeno-associated viruses). Further information on parvoviruses and other members of the Parvoviridae is described in Kenneth I. Berns, “Parvoviridae: The Viruses and Their Replication,” Chapter 69 in Fields Virology (3d Ed. 1996). For convenience, the present invention is further exemplified and described herein by reference to AAV. It is however understood that the invention is not limited to AAV but may equally be applied to other parvoviruses.

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural viral particle (VP) proteins. The VP proteins (VP1, -2 and -3) form the capsid. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wildtype (wt) AAV infection in mammalian cells the Rep genes (i.e. Rep78 and Rep52) are expressed from the P5 promoter and the P19 promoter, respectively, and both Rep proteins have a function in the replication and packaging of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production. The three capsid proteins, VP1, VP2 and VP3 are expressed from a single VP reading frame from the p40 promoter. wtAAV infection in mammalian cells relies for the capsid proteins production on a combination of alternate usage of two splice acceptor sites and the suboptimal utilization of an ACG initiation codon for VP2.

A “recombinant parvoviral or AAV vector” (or “rAAV vector”) herein refers to a vector comprising one or more polynucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such rAAV vectors can be replicated and packaged into infectious viral particles when present in an insect host cell that is expressing AAV rep and cap gene products (i.e. AAV Rep and Cap proteins). When an rAAV vector is incorporated into a larger nucleic acid construct (e.g. in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

The invention relates to a method for producing a recombinant parvoviral (rAAV) virion, comprising a recombinant parvoviral (rAAV) vector, in an insect cell. In one embodiment, the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an adeno associated virus (AAV). Preferably, the nucleotide sequences are of the same serotype. More preferably, the nucleotide sequences differ from each other in that they may be either codon optimized, AT-optimized or GC-optimized, to minimize or prevent recombination. Preferably, the difference in the first and the second nucleotide sequence coding for the common amino acid sequences of a parvoviral Rep protein is maximised (i.e. the nucleotide identity is minimised) by one or more of: a) changing the codon bias of the first nucleotide sequence coding for the parvoviral Rep common amino acid sequence; b) changing the codon bias of the second nucleotide sequence coding for the parvoviral Rep common amino acid sequence; c) changing the GC-content of the first nucleotide sequence coding for the common amino acid sequence; and d) changing the GC-content of the second nucleotide sequence coding for the common amino acid sequence. Codon optimisation may be performed on the basis of the codon usage of the insect cell used in the method of the invention, preferably Spodoptera frugiperda, as may be found in a codon usage database (see e.g. http://www.kazusa.or.jp/codon/). Suitable computer programs for codon optimisation are available to the skilled person (see e.g. Jayaraj et al., 2005, Nucl. Acids Res. 33(9):3011-3016; and on the internet). Alternatively, the optimisations can be done by hand, using the same codon usage database.

### Transgene

In one embodiment, the cell further comprises: a) a nucleotide sequence comprising parvoviral capsid protein coding sequences operably linked to a third promoter for expression in the insect cell; b) a nucleotide sequence comprising a transgene that is flanked by at least one parvoviral inverted terminal repeat sequence; and, c) a nucleotide sequence comprising an expression cassette for expression of the transcriptional transregulator.

In a further embodiment, the nucleotide sequences of at least one of a) and b) is comprised in a baculoviral vector, wherein, preferably the nucleotide sequences of at least one of a), b) and c) are comprised in the baculoviral vector comprising the expression cassette for expression of the transcriptional transregulator.

In the context of the invention “at least one parvoviral inverted terminal repeat nucleotide sequence” is understood to mean a palindromic sequence, comprising mostly complementary, symmetrically arranged sequences also referred to as “A,” “B,” and “C” regions. The ITR functions as an origin of replication, a site having a “cis” role in replication, i.e. being a recognition site for trans acting replication proteins, such as e.g. Rep 78 (or Rep68), which recognize the palindrome and specific sequences internal to the palindrome. One exception to the symmetry of the ITR sequence is the “D” region of the ITR. It is unique (not having a complement within one ITR). Nicking of single-stranded DNA occurs at the junction between the A and D regions. It is the region where new DNA synthesis initiates. The D region normally sits to one side of the palindrome and provides directionality to the nucleic acid replication step. A parvovirus replicating in a mammalian cell typically has two ITR sequences. It is, however, possible to engineer an ITR so that binding sites on both strands of the A regions and D regions are located symmetrically, one on each side of the palindrome. On a double-stranded circular DNA template (e.g., a plasmid), the Rep78- or Rep68-assisted nucleic acid replication then proceeds in both directions and a single ITR suffices for parvoviral replication of a circular vector. Thus, one ITR nucleotide sequence can be used in the context of the present invention. Preferably, however, two or another even number of regular ITRs are used. Most preferably, two ITR sequences are used. A preferred parvoviral ITR is an AAV ITR. More preferably AAV2 ITRs are used. For safety reasons it may be desirable to construct a recombinant parvoviral (rAAV) vector that is unable to further propagate after initial introduction into a cell in the presence of a second AAV. Such a safety mechanism for limiting undesirable vector propagation in a recipient may be provided by using rAAV with a chimeric ITR as described in US2003148506.

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence that is “flanked” by two other elements (e.g. ITRs), indicates that one element is located 5′ to the sequence and the other is located 3′ to the sequence; however, there may be intervening sequences there between.

In a preferred embodiment a nucleotide sequence of (i) is flanked on either side by parvoviral inverted terminal repeat nucleotide sequences.

In the embodiments of the invention, the nucleotide sequence comprising the transgene (encoding either a gene product of interest or comprising a nucleotide sequence targeting a gene of interest) that is flanked by at least one parvoviral ITR sequence preferably becomes incorporated into the genome of a recombinant parvoviral (rAAV) vector produced in the insect cell. Preferably, the transgene encodes a gene product of interest for expression in a mammalian cell. Preferably, the transgene comprises at least one nucleotide sequence targeting a gene of interest for silencing said gene of interest in a mammalian cell. Preferably, the nucleotide sequence comprising the transgene is flanked by two parvoviral (AAV) ITR nucleotide sequences and wherein the transgene is located in between the two parvoviral (AAV) ITR nucleotide sequences. Preferably, the nucleotide sequence encoding a gene product of interest (for expression in the mammalian cell) or comprising a nucleotide sequence targeting a gene of interest (for silencing the gene of interest in the mammalian cell) will be incorporated into the recombinant parvoviral (rAAV) vector produced in the insect cell if it is located between two regular ITRs, or is located on either side of an ITR engineered with two D regions.

AAV sequences that may be used in the present invention for the production of a recombinant AAV virion in insect cells can be derived from the genome of any AAV serotype. Generally, the AAV serotypes have genomic sequences of significant homology at the amino acid and the nucleic acid levels, provide an identical set of genetic functions, and produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). Any AAV serotype can be used as source of AAV nucleotide sequences for use in the context of the present invention. Preferably the AAV ITR sequences for use in the context of the present invention are derived from AAV1, AAV2, AAV4 and/or AAV7. Likewise, the Rep (Rep78/68 and Rep52/40) coding sequences are preferably derived from AAV1, AAV2, AAV4 and/or AAV7. The sequences coding for the VP1, VP2, and VP3 capsid proteins for use in the context of the present invention may however be taken from any of the known 42 serotypes, more preferably from AAV1, AAV2, AAV3, AAV4, AAVS, AAV6, AAV7, AAV8. AAV9, AAV10, AAV11, AAV12 or AAV13 or newly developed AAV-like particles obtained by e.g. capsid shuffling techniques and AAV capsid libraries, or from newly and synthetically designed, developed or evolved capsid, such as the Anc-80 capsid. AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., 1999, J. Virol., 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells.

The AAV capsid proteins, also known as VP proteins, are known to determine the cellular tropism of the AAV virion. The VP protein-encoding sequences are significantly less conserved than Rep proteins and genes among different AAV serotypes. The ability of Rep and ITR sequences to cross-complement corresponding sequences of other serotypes allows for the production of pseudotyped rAAV particles comprising the capsid proteins of a serotype (e.g., AAV3) and the Rep and/or ITR sequences of another AAV serotype (e.g., AAV2). Such pseudotyped rAAV particles are a part of the present invention.

Modified “AAV” sequences also can be used in the context of the present invention, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity (e.g., a sequence having about 75-99% nucleotide sequence identity) to an AAV1, AAV2, AAV3, AAV4, AAVS, AAV6, AAV7, AAV8, AAV9, AAV10, AAV11, AAV12 or AAV13 ITR, Rep, or VP can be used in place of wild-type AAV ITR, Rep, or VP sequences.

Although similar to other AAV serotypes in many respects, AAVS differs from other human and simian AAV serotypes more than other known human and simian serotypes. In view thereof, the production of rAAV5 can differ from production of other serotypes in insect cells. Where methods of the invention are employed to produce rAAV5, it is preferred that one or more constructs comprising, collectively in the case of more than one construct, a nucleotide sequence comprising an AAV5 ITR, a nucleotide sequence comprises an AAV5 Rep coding sequence (i.e. a nucleotide sequence comprises an AAV5 Rep78). Such ITR and Rep sequences can be modified as desired to obtain efficient production of rAAV5 or pseudotyped rAAV5 vectors in insect cells. E.g., the start codon of the Rep sequences can be modified, VP splice sites can be modified or eliminated, and/or the VP1 start codon and nearby nucleotides can be modified to improve the production of rAAV5 vectors in the insect cell.

In a preferred embodiment, an insect cell of the invention further comprises a nucleotide sequence comprising a transgene that is flanked by at least one parvoviral ITR sequence. Thus, preferably the nucleotide sequence comprises at least one AAV ITR and at least one nucleotide sequence encoding a gene product of interest (preferably for expression in a mammalian cell) or a nucleotide sequence targeting a gene of interest (preferably for silencing said gene of interest in a mammalian cell), whereby preferably the at least one nucleotide sequence encoding a gene product of interest or targeting a gene of interest becomes incorporated into the genome of an AAV produced in the insect cell. Preferably, the at least one nucleotide sequence encoding a gene product of interest is a sequence for expression in a mammalian cell. Preferably, the at least one nucleotide sequence targeting a gene of interest is a sequence for silencing said gene of interest in a mammalian cell Preferably, the nucleotide sequence comprises two AAV ITR nucleotide sequences and wherein the at least one nucleotide sequence encoding a gene product of interest or targeting a gene of interest is located between the two AAV ITR nucleotide sequences. Preferably, the nucleotide sequence encoding a gene product of interest (for expression in the mammalian cell) or the nucleotide sequence targeting a gene of interest (for silencing said gene of interest in a mammalian cell) will be incorporated into the AAV genome produced in the insect cell if it is located between two regular ITRs, or is located on either side of an ITR engineered with two D regions. Thus, in a preferred embodiment, the invention provides an insect cell according to the invention, wherein the nucleotide sequence comprises two AAV ITR nucleotide sequences and wherein the at least one nucleotide sequence encoding a gene product of interest or the at least one nucleotide sequence targeting a gene of interest is located between the two AAV ITR nucleotide sequences.

Typically, the transgene, including ITRs and promoter & polyadenylation sequences, is 5,000 nucleotides (nt) or less in length. In another embodiment, an oversized DNA molecule, i.e. more than 5,000 nt in length, can be expressed in vitro or in vivo by using the AAV vector described by the present invention. An oversized DNA is here understood as a DNA exceeding the maximum AAV packaging limit of 5.5 kbp. Therefore, the generation of AAV vectors able to produce recombinant proteins that are usually encoded by larger genomes than 5.0 kb is also feasible.

The nucleotide sequence comprising the transgene as defined herein above may thus comprise a nucleotide sequence encoding a gene product of interest (for expression in the mammalian cell) or the nucleotide sequence targeting a gene of interest (for silencing said gene of interest in a mammalian cell), and may be located such that it will be incorporated into an recombinant parvoviral (rAAV) vector replicated in the insect cell. In the context of the invention it is understood that a particularly preferred mammalian cell in which the “gene product of interest” is to be expressed or silenced, is a human cell. Any nucleotide sequence can be incorporated for later expression in a mammalian cell transfected with the recombinant parvoviral (rAAV) vector produced in accordance with the present invention. The nucleotide sequence may e.g. encode a protein or it may express an RNAi agent, i.e. an RNA molecule that is capable of RNA interference such as, e.g. an shRNA (short hairpinRNA) or an siRNA (short interfering RNA). “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). In a preferred embodiment, the nucleotide sequence comprising the transgene may comprise two coding nucleotide sequences, each encoding one gene product of interest for expression in a mammalian cell. Each of the two nucleotide sequences encoding a product of interest is located such that it will be incorporated into a recombinant parvoviral (rAAV) vector replicated in the insect cell.

The product of interest for expression in a mammalian cell may be a therapeutic gene product. A therapeutic gene product can be a polypeptide, or an RNA molecule (si/sh/miRNA), or other gene product that, when expressed in a target cell, provides a desired therapeutic effect. A desired therapeutic effect can for example be the ablation of an undesired activity (e.g. VEGF), the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof. Examples of therapeutic RNA molecule products include miRNAs effective in silencing diseases, including but not limited to polyglutamine diseases, dyslipidaemia or amyotrophic lateral sclerosis (ALS).

The diseases that can be treated using a recombinant parvoviral (rAAV) vector produced in accordance with the present invention are not particularly limited, other than generally having a genetic cause or basis. For example, the disease that may be treated with the disclosed vectors may include, but are not limited to, acute intermittent porphyria (AIP), age-related macular degeneration, Alzheimer's disease, arthritis, Batten disease, Canavan disease, Citrullinemia type 1, Crigler Najjar, congestive heart failure, cystic fibrosis, Duchene muscular dystrophy, dyslipidemia, glycogen storage disease type I (GSD-I), hemophilia A, hemophilia B, hereditary emphysema, homozygous familial hypercholesterolemia (HoFH), Huntington's disease (HD), Leber's congenital amaurosis, methylmalonic academia, ornithine transcarbamylase deficiency (OTC), Parkinson's disease, phenylketonuria (PKU), spinal muscular atrophy, paralysis, Wilson disease, epilepsy, Pompe disease, amyotrophic lateral sclerosis (ALS), Tay-Sachs disease, hyperoxaluria 9PH-1), spinocerebellar ataxia type 1 (SCA-1), SCA-3, u-dystrophin, Gaucher's types II or III, arrhythmogenic right ventricular cardiomyopathy (ARVC), Fabry disease, familial Mediterranean fever (FMF), proprionic acidemia, fragile X syndrome, Rett syndrome, Niemann-Pick disease and Krabbe disease. Examples of therapeutic gene products to be expressed include N-acetylglucosaminidase, alpha (NaGLU), Treg167, Treg289, EPO, IGF, IFN, GDNF, FOXP3, Factor VIII, Factor IX and insulin.

Alternatively, or in addition as another gene product, the nucleotide sequence comprising the transgene as defined herein above may further comprise a nucleotide sequence encoding a polypeptide that serves as a selection marker protein to assess cell transformation and expression. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel, supra. Furthermore, the nucleotide sequence comprising the transgene as defined herein above may comprise a further nucleotide sequence encoding a polypeptide that may serve as a fail-safe mechanism that allows to cure a subject from cells transduced with the recombinant parvoviral (rAAV) vector of the invention, if deemed necessary. Such a nucleotide sequence, often referred to as a suicide gene, encodes a protein that is capable of converting a prodrug into a toxic substance that is capable of killing the transgenic cells in which the protein is expressed. Suitable examples of such suicide genes include e.g. the E.coli cytosine deaminase gene or one of the thymidine kinase genes from Herpes Simplex Virus, Cytomegalovirus and Varicella-Zoster virus, in which case ganciclovir may be used as prodrug to kill the transgenic cells in the subject (see e.g. Clair et al., 1987, Antimicrob. Agents Chemother. 31: 844-849).

The nucleotide sequence comprising a transgene as defined herein above for expression in a mammalian cell, further preferably comprises at least one mammalian cell-compatible expression control sequence, e.g. a promoter, that is/are operably linked to the sequence coding for the gene product of interest. Many such promoters are known in the art (see Sambrook and Russel, 2001, supra). Constitutive promoters that are broadly expressed in many cell-types, such as the CMV promoter may be used. However, more preferred will be promoters that are inducible, tissue-specific, cell-type-specific, or cell cycle-specific. For example, for liver-specific expression (as disclosed in PCT/EP2019/081743) a promoter may be selected from an al-anti-trypsin promoter, a thyroid hormone-binding globulin promoter, an albumin promoter, LPS (thyroxine-binding globin) promoter, HCR-ApoCll hybrid promoter, HCR-hAAT hybrid promoter and an apolipoprotein E promoter, LP1, HLP, minimal TTR promoter, FVIII promoter, hyperon enhancer, ealb-hAAT. Other examples include the E2F promoter for tumor-selective, and, in particular, neurological cell tumor-selective expression (Parr et al., 1997, Nat. Med. 3:1145-9) or the IL-2 promoter for use in mononuclear blood cells (Hagenbaugh et al., 1997, J Exp Med; 185: 2101-10).

Various modifications of the nucleotide sequences as defined above, including e.g. the wild-type parvoviral sequences, for proper expression in insect cells is achieved by application of well-known genetic engineering techniques such as described e.g. in Sambrook and Russell (2001) “Molecular Cloning: A Laboratory Manual (3rd edition), Cold Spring Harbor Laboratory, Cold Spring Harbor Laboratory Press, New York. Various further modifications of coding regions are known to the skilled artisan which could increase yield of the encode proteins. These modifications are within the scope of the present invention.

### AAV

In the recombinant parvoviral (rAAV) vectors of the invention the at least one nucleotide sequence(s) encoding a transgene or gene product of interest for expression in a mammalian cell, preferably is/are operably linked to at least one mammalian cell-compatible expression control sequence, e.g., a promoter. As discussed in the above, many such promoters are known in the art.

AAV is able to infect a number of mammalian cells. See, e.g., Tratschin et al. (1985, Mol. Cell Biol. 5:3251-3260) and Grimm et al. (1999, Hum. Gene Ther. 10:2445-2450). However, AAV transduction of human synovial fibroblasts is significantly more efficient than in similar murine cells, Jennings et al., Arthritis Res, 3:1 (2001), and the cellular tropicity of AAV differs among serotypes. See, e.g., Davidson et al. (2000, Proc. Natl. Acad. Sci. USA, 97:3428-3432), who discuss differences among AAV2, AAV4, and AAV5 with respect to mammalian CNS cell tropism and transduction efficiency. In a preferred embodiment, a host cell of the invention is any mammalian cell that may be infected by a parvoviral virion, for example, but not limited to, a muscle cell, a liver cell, a nerve cell, a glial cell and an epithelial cell. In a preferred embodiment a host cell of the invention is a human cell.

### Method

In a further aspect, the invention provides for a method for producing a recombinant parvoviral virion. The method preferably comprises the steps of: a) culturing an insect cell as defined herein; b) providing the cell cultured in a) with the nucleotide sequences as defined herein; and, c) recovery of the recombinant parvoviral virion. In one embodiment, the cell culture in a) is transfected, also known as infected, with the nucleotide sequences as defined herein.

Recovery preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In a further embodiment, wherein recovery of the recombinant parvoviral virion in step c) comprises at least one of affinity-purification of the virion using an immobilised anti-parvoviral antibody, preferably a single chain camelid antibody or a fragment thereof, and filtration over a filter having a nominal pore size of 30-70 nm.

Therefore, in one embodiment the invention provides a method for producing a recombinant parvoviral virion in a cell. The method preferably comprising the steps of: a) culturing an insect cell as defined herein; b) infecting the cell cultured in a) with the nucleotide sequences as defined herein; and, c) recovery of the recombinant parvoviral virion wherein recovery of the recombinant parvoviral virion in step b) comprises at least one of affinity-purification of the virion using an immobilised anti-parvoviral antibody, preferably a single chain camelid antibody or a fragment thereof, or filtration over a filter having a nominal pore size of 30-70 nm.

In a further aspect the invention relates to a batch of parvoviral virions produced in the above described methods of the invention. A “batch of parvoviral virions” is herein defined as all parvoviral virions that are produced in the same round of production, optionally per container of insect cells.

In a preferred embodiment, the batch of parvoviral virions of the invention comprises a full virion:total virion ratio as described above and/or a full virion:empty ratio as described above.

### Kits

In a further aspect, the invention provides for a kit of parts comprising at least an insect cell as defined herein and a baculoviral vector and/or the nucleotide sequences as defined herein.

### DESCRIPTION OF THE FIGURES

FIGS. 1A-1C. (FIG. 1A): Schematic representation of transient transfection and baculovirus transactivation study involving reporter or pCLD expression construct. Luciferase activity was measured for the nano-luciferase reporter study and Western blotting was performed to determine Rep expression from the pCLD construct. GSG-P2A is a self-cleaving peptide (Wang et al., 2015). (FIG. 1B): Expression profiles of AAV Rep proteins from the indicated pCLD harbouring the indicated regulation element under the influence of different baculovirus transactivation at 48 hours post infection. T: Bac Trans. CT: Bac polH Cap Trans. C: Bac polH Cap. (FIG. 1C): The kinetics and intensity of reporter gene expression (nano-luciferase) regulated by the indicated promoter upon the indicated baculovirus transactivation. The relative luciferase units (RLU), a measure of luminescence, was measured out of 30 μl sample volume. Mock (circles): Inoculated by equal volume of fresh media. Bac Trans (squares): recombinant baculovirus harbouring only AAV ITR-transgene-ITR. Bac polH Cap Trans (triangles): recombinant baculovirus harbouring polH regulated AAV Cap gene and ITR-transgene-ITR. Bac polH Cap: recombinant baculovirus harbouring polH regulated AAV2 Cap gene only. Each data point represents an independent experimental replicate.

FIGS. 2A-2F. (FIG. 2A): Molecular design of the reporter constructs used to compare the enhancer activity of different homologous repeat (hr) elements in insect cells. The polH promoter was selected as the representative promoter. (FIG. 2B): The percentage of nucleotide similarity of all hr sequences in the baculovirus genome. (FIGS. 2C and 2D): The kinetics and intensities of reporter gene expression regulated by the indicated hr enhancer upon the indicated baculovirus transactivation. The relative luciferase unit (RLU) was measured in 30 μl sample volume. Each point represents an independent experimental replicate. (FIG. 2E): Molecular design of pCLD to compare the enhancer activity of different hrs on AAV Rep expression. (FIG. 2F): Expression profiles of the AAV Rep proteins under the influence of different hr enhancers at 48 hours post infection. T: Bac Trans.

FIGS. 3A-3B. (FIG. 3A): Alternative molecular designs of inducible single-Rep cassette plasmid vectors to minimize cis:trans promoter competition observed upon Bac polH Cap Trans transactivation. (FIG. 3B): Expression profiles of the AAV Rep proteins under the control of the indicated promotors in combination with the less leaky hr4b enhancer and stronger ATG start codon at 48 hours after the indicated baculovirus transactivation. T: Bac Trans. CT: Bac polH Cap Trans.

FIGS. 4A-4E. (FIG. 4A): The kinetics and intensities of reporter gene expression regulated by alternative baculovirus promoters upon the indicated baculovirus transactivation. (FIG. 4B): Molecular design of reporter constructs used to characterize alternative or suboptimal start codons for the 39k promoter. The hr2.09 enhancer was selected as the representative enhancer. (FIG. 4C): The kinetics and intensities of reporter gene expression with alternative start codons induced by the indicated baculovirus transactivation. (FIG. 4D): Molecular designs of pCLD constructs to observe AAV Rep expression profiles under the regulation of 39k promoter and the ACG start codon or (FIG. 4E): under additional promoter element within the artificial intron. The Western blot of the AAV Rep proteins from the indicated single-Rep cassette pCLD upon the indicated baculovirus transactivation. The relative luciferase unit (RLU) was measured out of 30 μl sample volume. Each data point represents an independent experimental replicate.

FIGS. 5A-5C. (FIG. 5A): The percentage of nucleotide similarity of codon optimized AAV2 Rep. (FIG. 5B): Molecular designs of inducible split-Rep cassette plasmid vectors harbouring the indicated regulation elements. (FIG. 5C): Expression profiles of AAV Rep proteins from the split-Rep cassette plasmid vectors upon the indicated baculovirus transactivation. ((+) and right arrow): forward orientation. ((−) and left arrow): reverse-complemented orientation. T: Bac Trans. CT: Bac polH Cap Trans.

FIGS. 6A-6E. (FIG. 6A): Schematic representation of the transient AAV production experimental set up. (FIGS. 6B and 6C): Genome copy titer (GC/m1) of nuclease resistant AAV particles in crude lysate buffer (CLB) harvested at 3 days post transient AAV production using the indicated pCLD transfection and baculovirus transactivation. Bac Cap5 FIX (circles): recombinant baculovirus harbouring polH regulated AAV Cap5 gene and ITR-FIX-ITR. Bac Cap2/5 nano-luciferase (triangles): recombinant baculovirus harbouring polH regulated AAV Cap2/5 (AAV2/5) gene and ITR-secreted-Nano-Luc-ITR. (FIGS. 6D and 6E): AAV Rep kinetics and expression profiles from the transient AAV production experiment. Each symbol represents an independent replicate of an AAV batch production.

FIGS. 7A-7C. (FIG. 7A): VP1:2:3 profile of AVB purified AAV2/5 nano-luciferase (FIG. 7B): Schematic representation of the AAV potency assay set up in Huh7 cells. The AVB purified AAV2/5 particles produced from the transient AAV production were normalized and used to inoculate Huh7 cells at a dose of 105 or 104 GC per cell. At 3 days post infection (d.p.i), the potency of AAV2/5 particle transduction was determined by quantification of nano-luciferase activity (relative luciferase units/RLU) secreted into the supernatant. luciferase (FIG. 7C): The potency comparison of purified AAV produced from transient AAV production using the indicated inducible Rep plasmid vectors (pCLD). Bac Rep183: BEV derived AAV material. Each symbol represents an independent replicate of an AAV batch production.

FIGS. 8A-7B. (FIG. 8A): (A) Molecular analysis of AAV DNA vector genomes, extracted from AAV batches produced from the indicated pCLD transient transfection experiment, on a denaturing formaldehyde agarose gel. The whole ITR-transgene-ITR size for the FIX and nano-luciferase vector genomes are 2,5 kb and 2 kb respectively. Black arrow: dimer replicative form of FIX or nano-15 luciferase vector genomes. White arrow: monomer replicative form. m: smart DNA ladder. High/low exp: gel exposure duration. (FIG. 8B): Hypothetical AAV total:full ratio (TF) based on total assembled AAV5 capsids/GC titer method. The number of total assembled capsids was determined by performing ELISA analysis on AVB purified AAV particles. The GC titer was also obtained from the same AVB purified particles.

FIGS. 9A-9C. (FIG. 9A): Schematic representation of the generation of novel stable iRep expresSf+ cell-lines. (FIG. 9B): Genome copy titer (GC/m1) and productivity (GC/input cell) of nuclease resistant AAV particles in crude lysate buffer (CLB) and harvested at 3 days post transient AAV production using the indicated iRep cell-lines and baculovirus transactivation. Each symbol represents an independent replicate of an AAV batch production. (FIG. 9C): AAV Rep kinetics and expression profiles from the transient AAV production experiment.

FIGS. 10A-10B. (FIG. 10A): Vp1:2:3 profile of AVB purified AAV2/5 sNano-Luc produced from the iRep cell-lines as indicated. (FIG. 10B): The potency comparison of the purified AAV particles in Huh7 cells. 3-ple Bac: AAV material produced from triple Bac production platform and Bac Rep: AAV material produced from Bac Cap Trans and Bac Rep combination in wild-type ExpresSf+ cells.

FIGS. 11A-11D. (FIG. 11A): List of AAVS FIX purified materials for particle quality study and the sourcing methodology. The iRep cell-lines replaced the use of wild-type ExpresSf+ cells and Bac Rep to produce the AAV materials. (FIG. 11B): Molecular analysis of AAV DNA vector genomes, extracted from the AAV materials, on a denaturing formaldehyde agarose gel. The whole ITR-transgene-ITR size for FIX vector genomes are 2.5 kb. Black and white arrows: dimer monomer replicative form of FIX. (FIG. 11C): Hypothetical AAV total:full ratio (TF) based on total assembled AAVS capsids/GC titer method. The total assembled capsids were determined by performing HPLC based analysis on the purified AAV particles. The GC titer was also obtained from the same particles. (FIG. 11D): Quantification of residual Bac DNA contamination in the purified AAV materials. The result is shown as ratio of Bac DNA per 1×10¹³ AAV GC.

FIGS. 12A-12F. Physical maps of plasmids used in this study: (FIG. 12A): pCLD 046; (FIG. 12B): pCLD 050; (FIG. 12C): pCLD 051; (FIG. 12D): pCLD 052; (FIG. 12E): pCLD 053; and (FIG. 12F): pCLD 054.

FIG. 13. The nucleotide alignment and size of all baculovirus hr sequences, including the synthetic hr sequence. The lines show the indicated hr sequences.

FIGS. 14A-14R. FIGS. 14A-14R: nucleotide alignment of coding sequences of wild-type Rep52, Rep183 Rep52 (as described in WO2009/014445) and an extreme-codon-optimized Rep52 coding sequence (SEQ ID NO: 15).

FIGS. 15A-15D. (FIG. 15A): Experimental set-up scheme for the validation of the iRep stable cell pool. Yellow indicated the pre-culture passages in 1L shake flask (passage 0-3). Green indicates the seed train production in 2L STR (passage 4-9). Red indicates the AAV production events in 1L shake flasks by transfection with baculovirus Bac CapS FIX passage 5 using the iRep stable cell pool at passage 5, 7, and 9 from the 2L STR. (FIG. 15B): Timeline for the validation of the iRep stable pool. The number in the brackets indicated the generation of the iRep stable pool cells used for transfection. (FIG. 15C): The expression of Rep 78 and Rep 52 in iRep stable pool cells at passage 5,7, and 9 at 48 and 72 hours post-transfection with Bac CapS FIX (P5). (FIG. 15D): The genome copies of transgene FIX in FCLB as a result of 72 hours post-transfection with Bac CapS FIX (P5). MOI indicates the volume ratio of Bac CapS FIX P5 used for the transfection of iRep stable pool cells. The number in the brackets indicated the passage number of the iRep stable pool cells used for transfection.

### EXAMPLES

### Materials and Methods

### Cell Culture

Huh7 cells were maintained in Dulbecco's modified Eagle's medium (DMEM, Invitrogen) supplemented with 10% (v/v) fetal bovine serum (FBS) at 37° C., 5% CO2. Sf9 and ExpresSf+ cells were maintained in Sf-900 II SFM (Gibco) in shaker flasks at 28° C., 135 rpm. In the case of Sf9 cells, the cultured cells were supplemented with 10% FBS (Gibco).

### Inducible Expression Plasmids and Recombinant Baculovirus Construction

All inducible expression plasmid series (pCLDs) and the nano-luciferase reporter constructs were created using GeneArt gene synthesis services (ThermoFisher). To generate the recombinant baculovirus comprising the ITR-transgene-ITR only (Bac Trans) or AAV Cap expression cassette only (Bac polH Cap2/5; Urabe, M. et al., 2006) or both AAV Cap expression cassette and ITR-transgene-ITR (Bac polH CapS—human Factor IX or Bac polH Cap2/5—secreted Nano-luciferase [nano-luciferase]), Sf9 cells were transfected with either pVD-ITR-transgene-ITR (SEAP transgene) (SEQ ID NO. 01) or pVD-poIH-Cap (polH Cap2) (SEQ ID NO. 02) or pVD-poIH-Cap-ITR-transgene-ITR (polH Cap Trans) (CapS FIX: SEQ ID NO. 03, Cap2/5 nano-luciferase: SEQ ID NO. 29) and linearized baculovirus genome by using Cellfectine II reagent. Then, the positive cell plaques were transferred into adherently cultured Sf9 cells. At 72 h post transfection, the infected supernatant from Sf9 cells was further passaged and amplified in ExpresSf+ cells until reaching passage 4 (P4). After analysing the recombination event and genome stability, the P4 material from the selected recombinant baculovirus was stored in liquid nitrogen as aliquots and only freshly amplified to P5 working seed virus prior to characterization experiments. The baculovirus expressing AAV2 Rep (Bac Rep183) (SEQ ID NO. 04) was generated as explained previously (Urabe, M. et al., 2006). This Bac Rep183 is also referred to a as the split-cassette AAV Rep, or split Rep.

### AAV Vector Production

The AAV variants were generated by infecting the transiently transfected ExpresSF+ insect cells with freshly amplified recombinant baculovirus stocks (P4→P5) comprising the indicated AAV Cap and transgene (Urabe, M. et al., 2002). After 72 h incubation at 28° C., cells were lysed with 1% Triton X-100 for 1 h. Genomic DNA was digested via benzonase (Merck) treatment for 1 h at 37° C., and cell debris was removed by centrifugation for 15 min at 1900×g. The clarified lysate was stored at 4° C. until the start of the purification, and DNase-resistant AAV particle titers were determined using quantitative polymerase chain reaction (qPCR) with primers and probe directed against the promoter region of the indicated transgene (see Table 1). To purify the AAV vector, the clarified lysate was purified using AVB Sepharose (GE Healthcare). The purified virus titers were then determined by qPCR.

TABLE 1
Primer and probe sequences for qPCR.

| Primer/Probe for qPCR | Sequence |
|---|---|
| CMV Primer for | 5′-TTGACGCAAATGGGCGGTA-3′ |
| CMV Primer rev | 5′-GATCTGACGGTTCACTAAACGAG-3′ |
| CMV Probe | FAM-5′ TATAGACCT-ZEN-CCCACCGTACACGCC-3′- |
| | IBFQ |
| FIX Primer for | 5′-CAAGTATGGCATCTACACCAAAGTCT-3′ |
| FIX Primer rev | 5′-GCAATAGCATCACAAATTTCACAAA-3′ |
| FIX Probe | FAM-5′-TGTGAACTGGATCAAGGAGAAGACCAAGC-3′- |
| | IBFQ |

### Transient Transfection and Expression Analysis

To analyse protein expression, ExpresSf+ cells were adherently seeded and transfected with 1 pg of plasmid DNA encoding either the inducible Nano-luciferase reporter or Rep gene. The Cellfectin II Reagent (Invitrogen) was used for the transfection. One day after transfection, the indicated P5 baculovirus at 1% (v/v) end concentration was inoculated.

### SDS-Page and Western Blot

Western blot analysis was performed with cell lysates from the transfected cells lysed with RIPA buffer (Sigma Aldrich)+protease inhibitor cocktails (Roche) at 48 h post transfection. Cell lysates were loaded into mini-protean precast 4-12% bis-tris polyacrylamide gels (BioRad) in equal volume. The gels were then blotted into ready to use PVDF membranes using trans-blot turbo transfer system (BioRad). The membranes were then incubated with α-AAV2-Rep (Progen, Germany), followed by incubation with secondary antibody coupled to horseradish peroxidase (HRP) (Sigma-Aldrich). Bound antibodies were detected with the ECL detection system (Thermo Pierce) and imaged via Chemidoc imager (BioRad). For VP protein imaging, protein composition of purified AAV particles was determined by electrophoresis on mini-protean stain-free ® precast 4-12% bis-tris polyacrylamide gels (BioRad). The gels were then put into Chemidoc imager and the image was analysed with image lab software (BioRad).

### In Vitro Potency

Huh7 cells were infected with AAV variants expressing secreted Nano-luciferase as the transgene at different MOI's (in GC/cell). Co-infection with wild type Adenovirus (MOI 30) was performed to stimulate second strand synthesis. Forty-eight hours after the start of the infection, secreted Nano-luciferase expression was measured in the supernatant using the assay kit and Glomax luminometer (Promega) with an integration time of 1 s.

### Formaldehyde Gel Electrophoresis with Genomic AAV DNA

Genomic AAV DNA was isolated from purified AAV batches with the PCR purification Nucleospin kit (Machery Nagel). Prior to electrophoresis, 500 ng of AAV genomic DNA was denatured for 10 minutes at 95° C. in formaldehyde loading buffer (1 ml 20× MOPS, 3.6m1 37%

Formaldehyde, 2 ml 5mg/ml Orange G in 67% sucrose, to 10m1 with MQ) and immediately put on ice. Next, samples were run on a 1% agarose gel made in 1× MOPS buffer (40 mM MOPS, 10 mM NaAc, 1mM EDTA, pH=8.0) supplemented with 6.6% formaldehyde. Samples were then run for 2 hours at 100 volts in 1× MOPS buffer supplemented with 6.6% formaldehyde running buffer. After the run, DNA was stained with SYBR Gold (Thermofisher) and bands were visualized on a Chemidoc touch imager (Biorad).

### Hypothetical Total:Full Ratio Measurement

Hypothetical total to full ratios (T/F) for the productions were calculated by dividing the total amount of assembled capsids with the GC amount (measured by qPCR) of respective AVB purified

AAV materials. To measure total capsid or total particle, ELISA or HPLC based analysis was performed. AAV Titration ELISA kit (Progen, Germany) was used to quantify full virions and assembled empty capsids of AAVS. The capture-antibody detects a conformational epitope not existing on unassembled or individual capsid VP proteins. The AVB purified AAV materials were diluted 1000-2000 fold in the kit's assay buffer. The experiment was performed according the kit's protocol.

Size exclusion chromatography was also used to determine the total AAVS particle content. The method uses an HPLC system with a BioBasic SEC-1000 column, which is chosen for its capacity to separate larger particles such as AAV. The AAV particles are detected at an absorbance of 214 nm. A working standard (WS), being an AAVS-based product with a known total particle content (verified against the initial reference standard), was used to generate a calibration curve (total particle concentration versus peak area). The peaks were integrated and quantified using the Chemstation-software. AAVS samples are quantified against this calibration curve.

### Residual Baculovirus DNA Quantification

Residual baculovirus DNA is present as a process-related impurity in AAV Drug Substance-and Drug Product preparations. Residual baculovirus DNA levels are assessed by qPCR using a primer set specific fora representative region in the baculovirus genome (close to the HR3 enhancer region).

### Generation of ExpresSf+ Stable Cells with Inducible Expression of AAV2 Rep (iRep Sf+ cells)

The parental ExpresSf+ cells harboring all cells were passaged 1 day prior to the plasmid DNA transfection. On the day of transfection, the parental cells were diluted with fresh pre-warmed Sf-900 II media into 1.5×10⁶ cells/ml density and then returned back into shaker incubator until cell seeding. Transfection mix of DNA (1 pg dna/cell) : liposome (Cellfectine II) complex in 1 ml saline solution was prepared. While waiting for the complex formation, the diluted cells were taken out and distributed as 7.5×10⁶ cells in 5 ml volume in each designated 125 ml shake flask. The DNA:liposome complex mix was added by slowly dropping the whole 1 ml complex volume on top of the cells in 125 ml shake flask followed by gently swirling to homogenize the complex and 5 hour incubation in the shaker incubator at 28° C., 135 rpm, and without CO₂. After 5 hours, another 9 ml of fresh Sf-900 II media was added and the transfected cells were further incubated. Three days after, the cells were spun down by centrifugation, the old media was discarded by decantation and replaced with fresh Sf-900 II media to dilute the whole cell pellets into 5×10⁵ cells/mi end cell density. The blasticidin antibiotic selection pressure was added into the cell suspension at the end-concentration of 25 μg/ml. After the cell viability has reached above 90% (±in 3 weeks), the stably transfected cells were passaged normally but with the continuous presence of blasticidin.selection pressure. As soon as the cell viability is >95% and the doubling time is ±24-26 hours or lower, cell pool banking is performed with at least 30 cryotubes per-stable cell pool.

### iRep Stable Pool Pre-Culture Setting for Sequential Batches Reactors (SBR) Study

The pre-cultures (PO-P3) of iRep 052 (iRep) stable pool cells were produced as usual in shake flask. A 1.5 L of fresh SF900 It medium (Thermo Fisher Scientific) was added into the 2L STR and was equilibrated the temperature to 28.0° C. The DO sensor and the cell density probe (Incyte Arc, Hamilton) of the bioreactor was re-calibrated with the pre-warm medium. The P4 pre-culture from the shake flasks was pooled and was measured for the viable cell density (VCD) using NucleoCounter NC-100 according to GEN-SOP-0031—Operation of BucleoCounter NC-100. A calculated volume of pooled P3 culture and a calculated volume of additional fresh SF900II medium (Thermo Fisher Scientific) were transferred to the 2L bioreactor (UniVessel® SU, Sartorius) at the final viable cell density of 0.5e6 VC/mL with a final working volume of 2 L. The cultivation was carried out at temperature of 28 ° C. which was maintained by a thermo-mat surrounding the reactor vessel. Compressed air was continuously gassing to the reactor at a flow rate of 5 cubic centimeters per minute (ccm) and with an air overlay of 0.30 liter per minute (lpm). The dissolved oxygen concentration and pH was measured on-line by a built-in electrochemical sensor interfaces. The oxygen supply of the reactor was maintained at 30% saturation of dissolved oxygen by a cascade control of gassing oxygen to the reactor in combination with a cascade control of stirrer (Table 2). The gains of the proportional, integral and derivative (PID) settings of the 2 L bioreactor for the controller of temperature and oxygen indicated on (Table 3). The cell density of the culture was measured on-line by a cell density probe during the cultivation.

After 48-72 hours cultivation and according to the VCD of P4 preculture, a calculated volume of pre-culture P4 was drained from the bottom of the bioreactor, and a calculated volume of fresh SF900 II medium (Thermo Fisher Scientific) was subsequently added into the bioreactor by the effluent pump until the final viable cell density of 0.5e6 VC/mL at the final working volume of 2L. This filling and drawn cycle of SBR repeated to the cell culture of passage number up to 9.

TABLE 2
Cascade control setting for 2 L STR at 2 L working volume.

| | Oxygen gasflow at micro | |
|---|---|---|
| Output % | sparger (ccm) | Stirrer (rpm) |
| 0 | 0 | 200 |
| 20 | 0 | 275 |
| 40 | 0 | 335 |
| 60 | 0 | 335 |
| 80 | 35 | 335 |
| 100 | 75 | 335 |

TABLE 3
PID settings for the controllers of
oxygen and temperature on 2 L STR

| Setting | Oxygen controller | Temperature controller |
|---|---|---|
| XP (%) | 150 | 15 |
| TI (sec) | 400 | 999 |
| TD (sec) | 0 | 75 |
| MIN (%) | 0 | 0 |
| MAX (%) | 100 | 40 |
| DEADB (C) | 0 | 0 |

### Results

### Example 1: The Use of Alternative Late Baculovirus p10 Promoter in Inducible Plasmid Vectors Ameliorates Cis:Trans Competition Caused by the Incorporation of polH Promoter in Recombinant Baculovirus

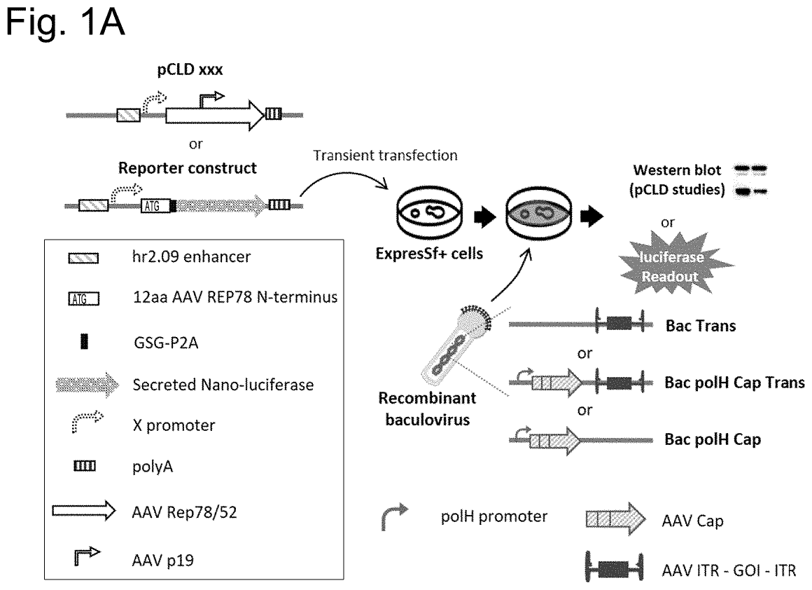

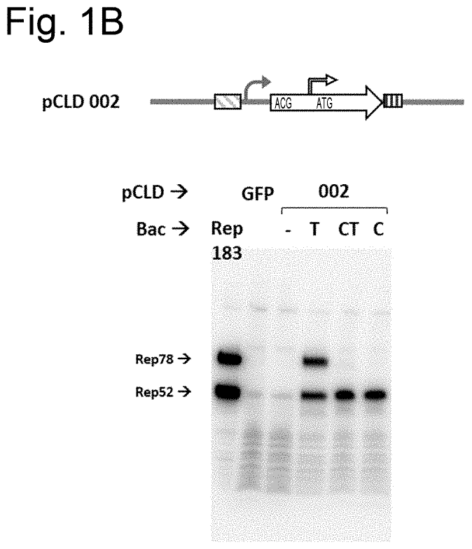

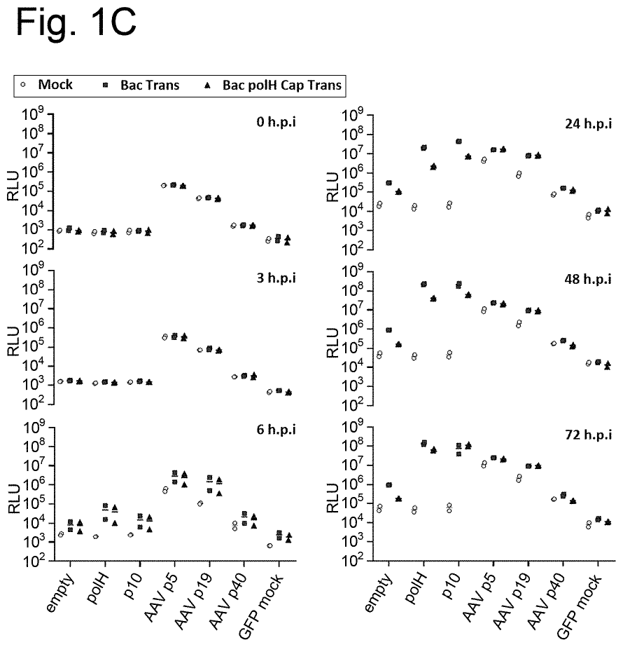

The use of late promoters, especially polH (SEQ ID NO. 25), as a recombinant promoter has become a conservative strategy to regulate the expression of recombinant genes in the BEV system. Therefore, the same strategy is also commonly used and optimized for AAV production using the BEV system (Urabe, M. et al., 2002). A similar strategy has also been implemented in the generation of 1st stable and inducible AAV packaging cells (Aslanidi, G., et al., 2009, supra). To generate these stable cells, both AAV single-cassette Rep and Cap expression plasmids regulated by hr2.09 and late polH promoter were used and stably integrated into the host insect cell genome. Interestingly, Wu et al. (supra) have recently shown the next generation of AAV packaging cells with increased flexibility by letting the AAV Cap expression to be driven by the recombinant baculovirus instead of the packaging host cells (Wu, Y. et al. 2019). Nonetheless, it is unclear if the use of a conservative late promoter, especially polH, within the recombinant baculovirus genome would interact with, or even interfere with, the same promoter in the integrated expression plasmids during transactivation. To elucidate this, an inducible expression plasmid vector (pCLD 002) (SEQ ID NO. 05) was designed with an upstream hr2.09 enhancer combined with full-length AAV2 Rep with attenuated ACG start codon (SEQ ID NO. 18) (Hermens, W. T. J. M. C., et al., 2009). This pCLD 002 was transiently transfected into ExpresSf+ cells (FIG. 1A) followed by transactivation via inoculation of different recombinant baculoviruses. Intriguingly, the expression of AAV2 Rep78 could only be observed when transactivation was done using Bac Trans (FIG. 1B). The other baculoviruses, both Bac polH Cap and Bac polH Cap Trans, could only induce the expression of AAV2 Rep52 suggesting that the expression of Rep78 and Rep52 is regulated differently within the single AAV Rep cassette design. To confirm this finding, nano-luciferase reporter constructs were designed with an upstream hr2.09 enhancer combined with a conservative late promoter (either polH or p10 (SEQ ID NO. 22)) and a similar experiment, as described previously, was performed (FIG. 1A). Using this approach, we elucidated the reporter induction profile upon transactivation by inoculation of different recombinant baculoviruses, either with (Bac polH Cap Trans) or without (Bac Trans) recombinant conservative late promoters (FIG. 1A). To show there was no difference in infectivity between recombinant baculoviruses, native AAV2 promoters (p5, p19, and p40) were also tested. No significant difference was observed in the induction profiles of the three AAV promoters when samples were inoculated with either Bac Trans or Bac polH Cap Trans (FIG. 1C, squares vs triangles) indicating there was similar infectivity between these recombinant baculoviruses. Interestingly, we noted a stronger induction of reporter expression upon Bac Trans transactivation when Nano-luciferase is regulated by polH and p10, as early as 24 hours after infection. At the same time point, the transactivation by Bac polH Cap Trans exhibited ±10 (polH) and ±5-fold (p10) lower reporter gene expression as compared to Bac Trans (FIG. 1C). At later time points, the difference in induction between Bac polH Cap Trans or Bac Trans became less. Furthermore, the p10 promoter reporter showed slightly stronger upregulation (±2-fold) as compared to the polH upon Bac polH Cap Trans transactivation (FIG. 1C). The results from pCLD 002 and reporter construct studies indicate that there is an interaction, or competition, between the polH promoter in the expression plasmid vectors and the recombinant baculovirus during transactivation. Remarkably, insertion of the alternative late p10 promoter in the expression plasmid vectors can ameliorate the transactivation competition.

### Example 2: The Use of Alternative Baculovirus Hr Enhancers Reduce Basal Gene Expression and Convey Tight Regulation in Baculovirus Transactivatable Plasmid Vectors

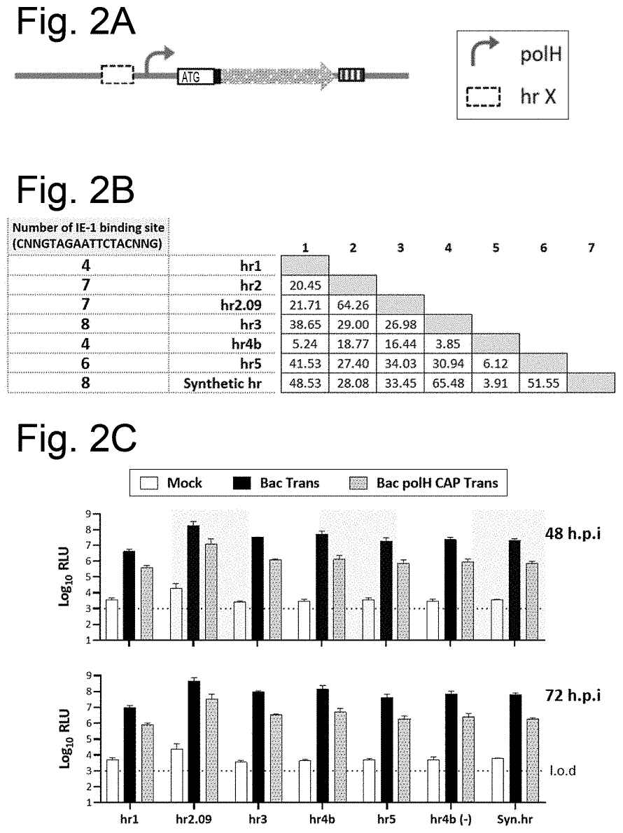

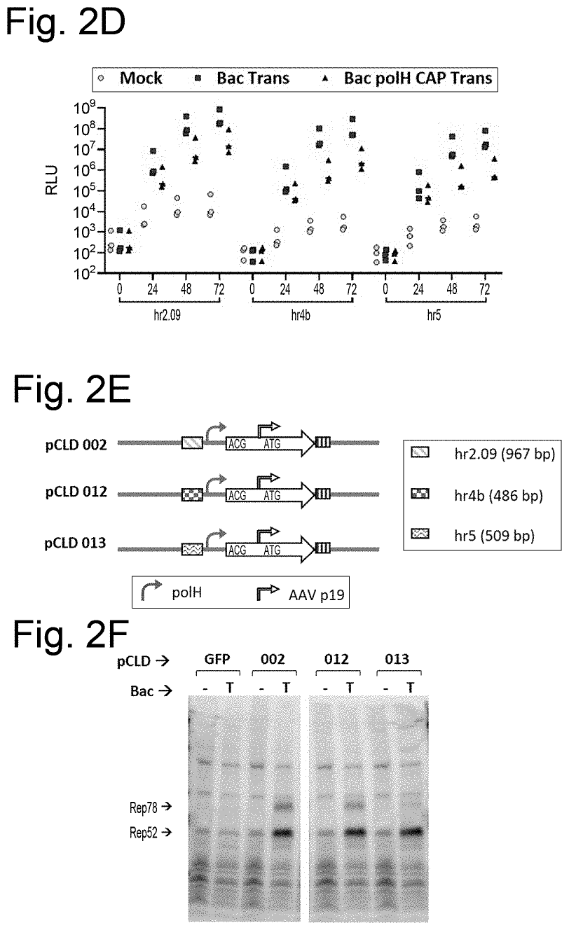

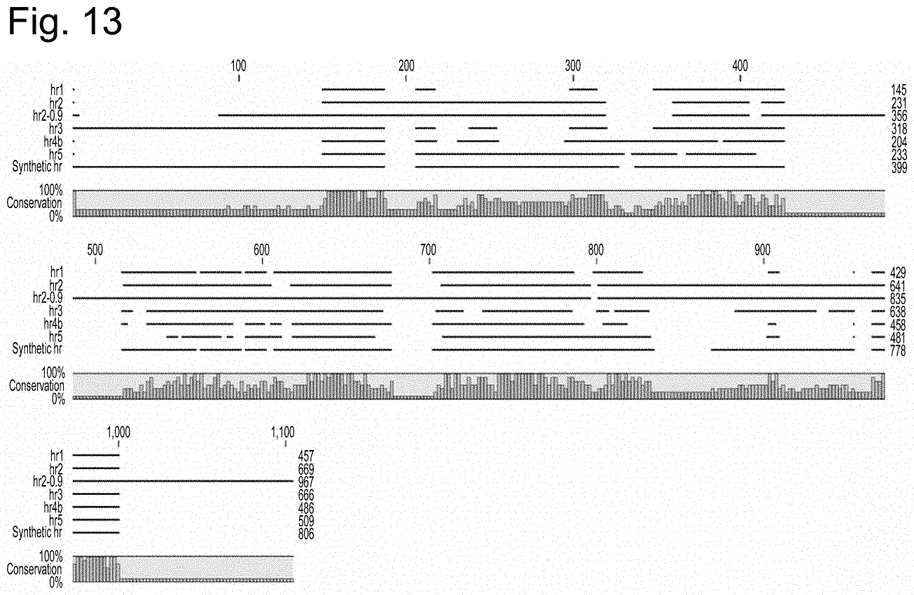

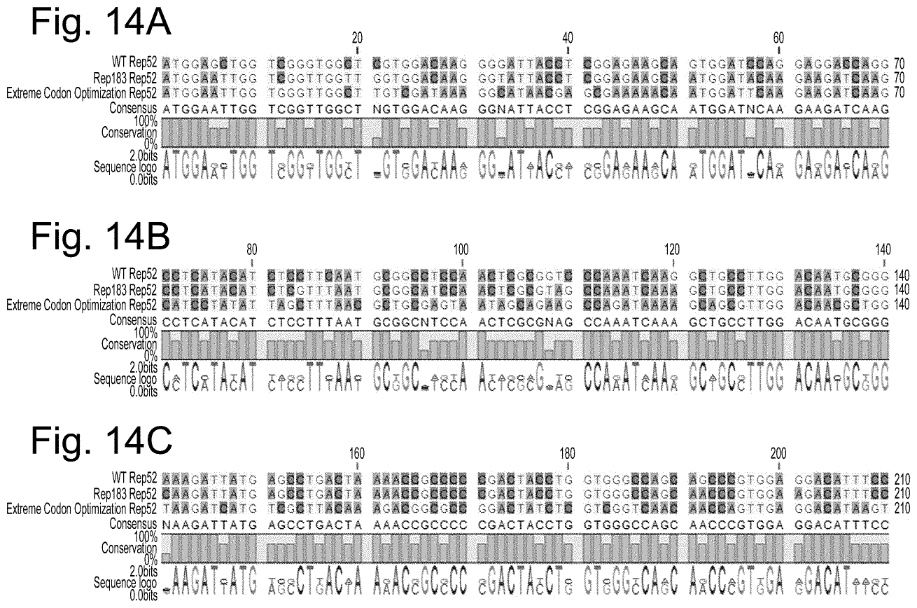

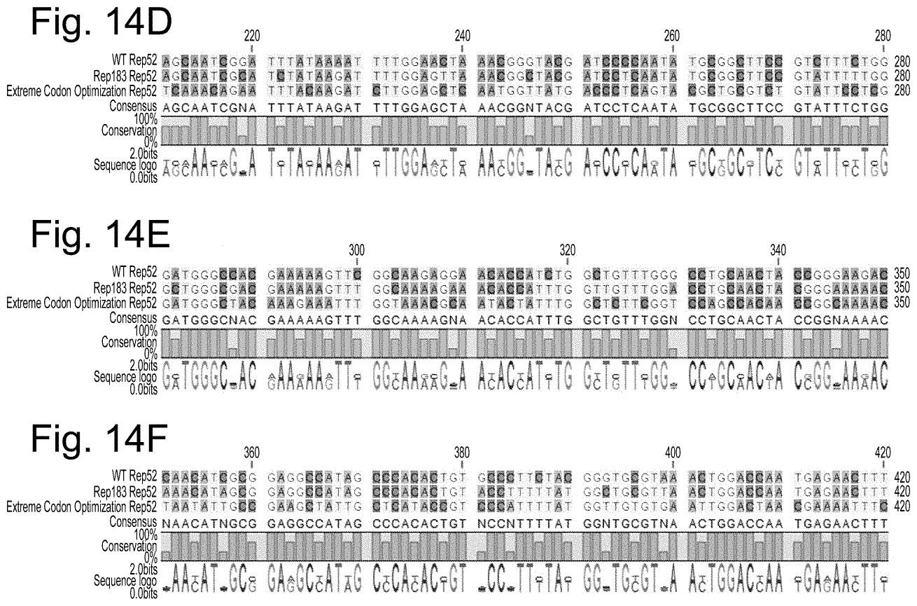

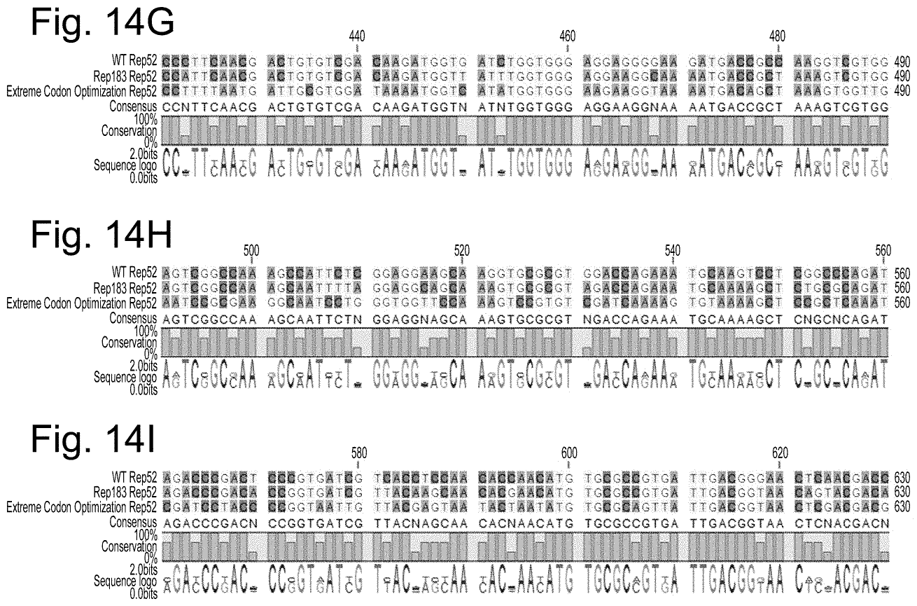

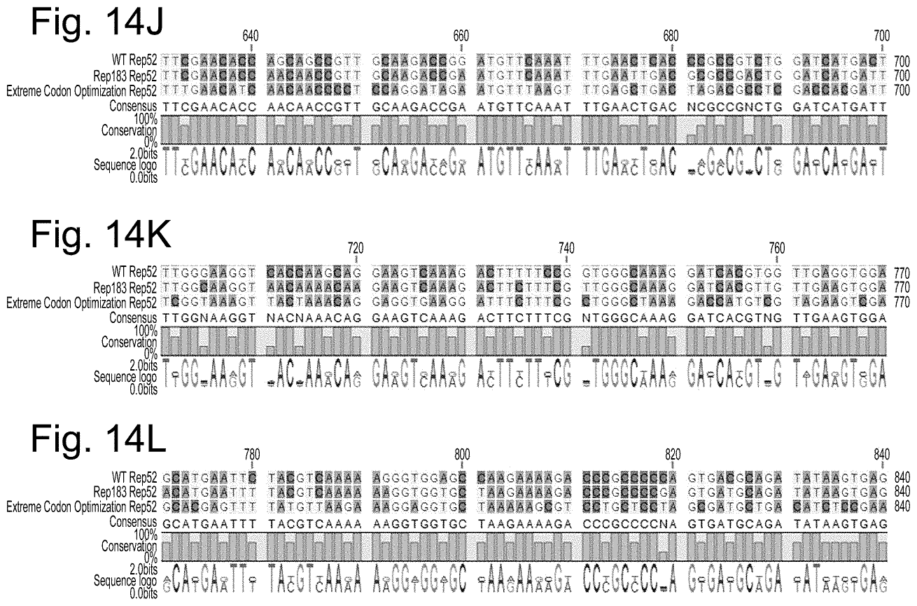

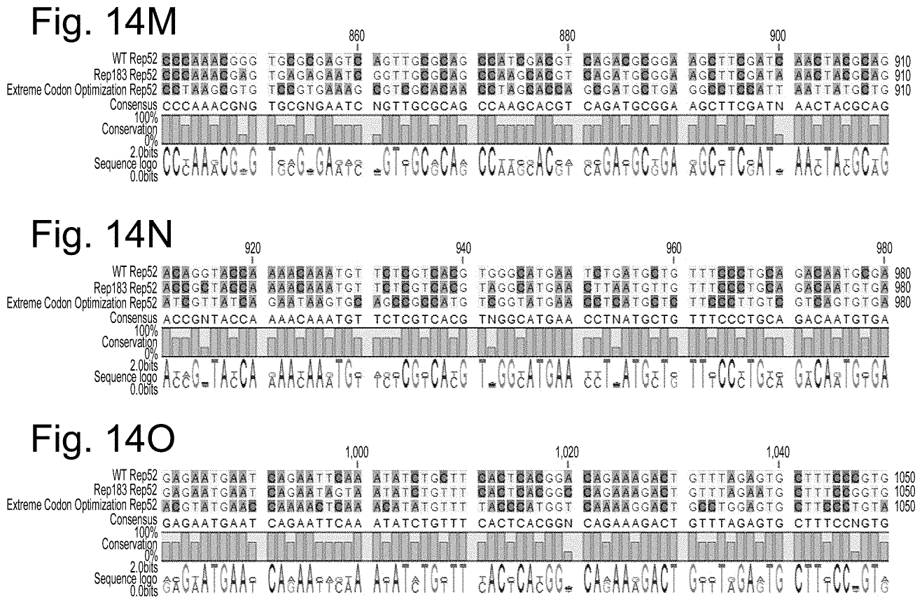

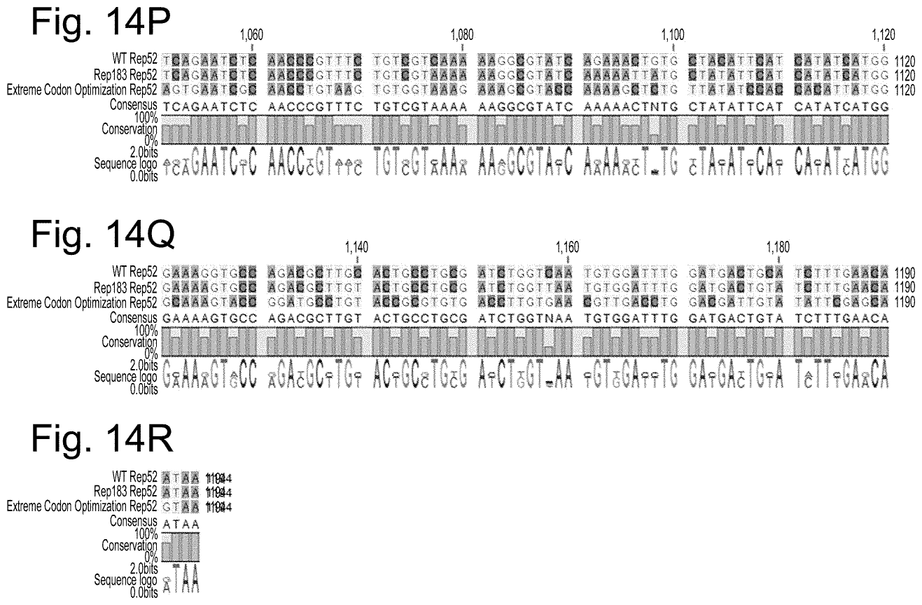

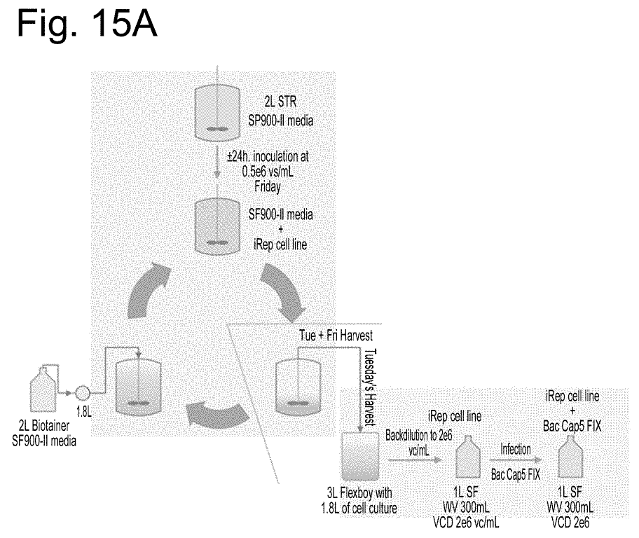

Various baculovirus hr sequences have been shown to possess transcription enhancer activity (Bleckmann, M. et al. 2016; Rodems, S. M. & Friesen, P. D., 1993; Venkaiah, B., et al., 2004). Together with various baculovirus promoters, this hr function has been exploited to create recombinant expression plasmids in insect cells. Similar strategies have also been used to generate baculovirus transactivatable AAV gene expression plasmid vectors. The use of hr2, or hr2.09 to be more precise, has been shown to strongly enhance both AAV Rep and Cap expression from plasmid vectors upon transactivation with recombinant baculovirus (Aslanidi, G., et al., 2009, supra). A total loss of gene expression was observed in the absence of hr sequence indicating the necessity of its presence for transactivation. It is known that the presence of the IE-1 DNA binding site sequence (CNNGTAGAATTCTACNNG) within the hr is responsible for its enhancer function (Olson, V. A., et al., 2003). In this example, the enhancer capacity of hr2/hr2.09 (having 7× IE-1 DNA binding sites) and others (i.e. hr1 [SEQ ID NO 26], hr3 [SEQ ID NO 27], hr4b [4x IE-1 DNA binding sites, SEQ ID NO. 19] and hr5 [6× IE-1 DNA binding sites, SEQ ID NO. 20]) combined with polH, as reference promoter, was profiled using the nano-luciferase reporter constructs upon transactivation with different recombinant baculoviruses (FIGS. 1A & 2A). These hr enhancers have significant nucleotide differences amongst each other, despite the intermittent presence of the IE-1 DNA binding sites (FIGS. 2B). We could see that all hr sequences, regardless of their directionality, indeed did enhance the promoter activity upon transactivation with baculoviruses, despite differences in degrees of activity (FIG. 2C). Interestingly, the synthetic hr, engineered to have 8 IE-1 DNA binding sites, together with the hr3 failed to enhance the promoter expression beyond the hr2.09 which has only 7 sites. This proves the amount of the binding site does not correlate with the enhancer strength as previously postulated (Aslanidi et al., 2009, supra). Through this experiment, hr2.09 was still observed to exhibit the highest enhancing function, followed by hr4b, which is one the shortest hr sequences with the fewest IE-1 binding element (FIGS. 2B and 13). However, some degree of leaky expression could also be observed from the mock treated hr2.09 sample as compared to the other hr sequences (FIGS. 2C and D). This leaky expression observed for hr2.09 could pose an issue especially when it is used to regulate a very toxic protein, such as AAV Rep. To compare these distinct hr enhancers on AAV Rep expression, several plasmid vectors (pCLDs) were made and tested for regulation of expression upon baculovirus transactivation (FIGS. 1A and 2E). Indeed, from the Western blot results (FIG. 2F), all hr enhancers could be used to enhance the polH function on AAV2 Rep upregulation. Interestingly, distinct expression strength, with hr2.09>hr4b>hr5, could only be observed significantly on the AAV2 Rep78, but not on the smaller Rep52 expression. This could indicate the presence of a distinct regulation system within the single AAV Rep cassette, where the native endogenous AAV p19 promoter is functional in the presence of any hr and inducible regardless of baculovirus addition. The leaky expression of hr2.09, which was observed when measuring luciferase activity, was not observed by Western blot, with the use of the already significantly attenuated ACG-Rep78 version as the sensitivity with this method compared to the nano-luciferase reporter assay could be the main issue to yield the right observational outcome. Thus, the use of alternative hr enhancers, especially relatively weaker hrs such as hr4b and hr5, can be used to overcome the leaky expression issue of the inducible AAV gene expression plasmid vectors using hr2.09.

### Example 3: The Use of Non-Leaky hr Enhancers, Late Promotor p10, and a STRONG ATG START CODON Conveys an Optimum Single-Cassette Rep Design Inducible by Baculoviruses Harbouring Recombinant polH Promoter

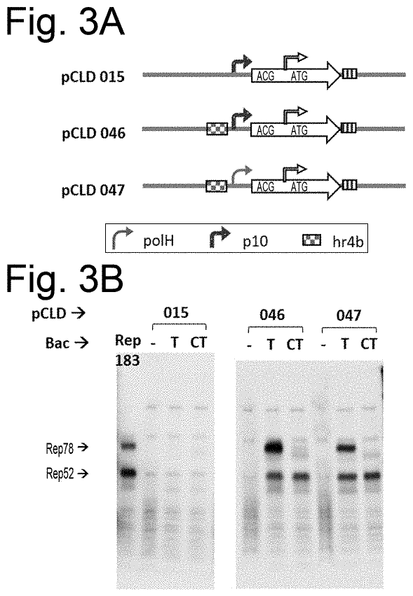

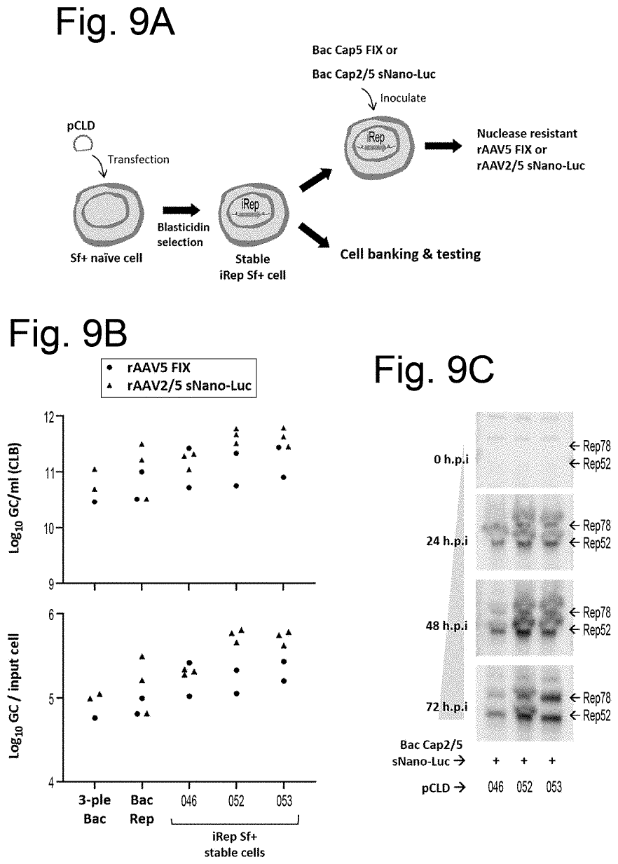

As shown by the previous example, the use of the polH promoter in combination with an attenuated ACG start codon can bring a seemingly normal AAV2 Rep expression ratio (low Rep78 and high Rep52) upon transactivation with Bac Trans (Urabe et al., 2006; Hermens et al., 2007). However, when using Bac polH Cap Trans for induction a relatively weaker transactivation profile is observed due to i) the cis:trans promoter competition between the two polH promoters used (for Cap in the Bac polH Cap Trans and for Rep in the expression plasmid) and ii) the adoption of non-leaky but relatively weaker hr such as hr4b. In order to create a non-leaky expression platform that is still compatible with the use of Bac polH Cap Trans, the hr4b enhancer was combined with the p10 promoter to regulate single-cassette AAV2 Rep with a strong wild-type ATG start codon (FIGS. 3A and 9) (SEQ ID NO. 06, SEQ ID NO. 17). To compare and confirm reduction of the cis:trans promoter competition during transactivation by recombinant baculovirus inoculation, a plasmid vector with the conservative polH promoter was created (FIG. 3A, pCLD 047) (SEQ ID NO. 07). These expression plasmids were transfected and transactivated following the previous experimental design (FIG. 1A). As can be seen from pCLD 015 (SEQ ID NO. 08) Western blot results, both the p10 and the endogenous AAV p19 promoter were shown to require the presence of a hr enhancer during its transactivation. The use of these constructs (both pCLD 046 and 047) together with Bac Trans transactivation yielded a suboptimal AAV Rep expression ratio (too high Rep78), presumably due to the adoption of a strong ATG start codon. However, upon Bac polH Cap Trans transactivation, proper expression of Rep78 and whole Rep ratio could be reached using the p10 promoter, but not with the polH. Intriguingly, there was no difference in the expression of Rep52 regardless of the baculovirus used for transactivation confirming the distinct regulation between upstream promoter vs. endogenous AAV p19 promoter within the single-cassette AAV Rep which is in coherence with the previously reported results (FIG. 1B).

### Example 4: The Use of a Non-Leaky hr Enhancer in Combination with Alternative Baculovirus Promoters to Inducibly Regulate a Split-Cassette AAV Rep Design

As shown by the previous example, the use of a recombinant promoter within the baculovirus genome (i.e. Bac polH Cap Trans) elicits a different expression profile of the reporter gene due to cis:trans promoter competition. This would be problematic, especially when adapting the AAV2 split Rep-cassette to an inducible expression plasmid design, as this entails the use of two polH promoters. Within the BEV split-cassette Rep (Bac Rep183), the expression of Rep78 and Rep52 fall under the regulation of a truncated immediate early IE-1 promoter (ΔIE-1) and late polH promoter, respectively (Urabe, M. et al., 2002; Hermens et al., 2007; Hermens et al., 2009). The effort to adopt this design to baculovirus transactivatable plasmid vectors has been previously attempted with unsuccessful outcome, presumably due to the constitutive nature of ΔIE-1 promoter and cis:trans competition of polH promoter in the tested design (Aslanidi, G., et al., 2009). The split-cassette Rep has become the fundamental AAV Rep cassette design in BEV platform because of the superior AAV quality that it can yield (Urabe, M. et al., 2002; Hermens, W. T. J. M. C., 2009). The superiority of split-cassette Rep is also presumably due to the possible expression intensity and temporal control that this design offers. In contrast, the single-cassette Rep design is more rigid and the expression of small Rep52 upon transactivation is known to be biasedly regulated by the endogenous AAV p19 promoter (FIGS. 2E & 3B) restricting its temporal expression to an early time point during infection (FIG. 1C). Moreover, scrutinizing the promoter reporter study (FIG. 1C), the AAV p19 promoter was found to be relatively leaky resulting in constitutive low expression of Rep52 which, in the long run, leads to toxicity for host cells. This would make the single-cassette Rep plasmid vectors less ideal despite the adoption of less leaky hr enhancer.

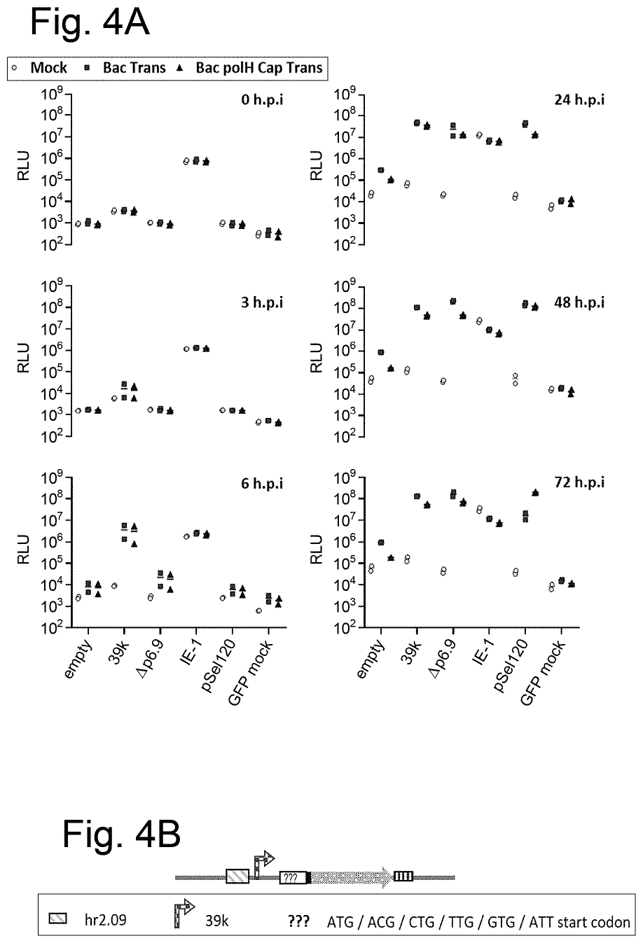

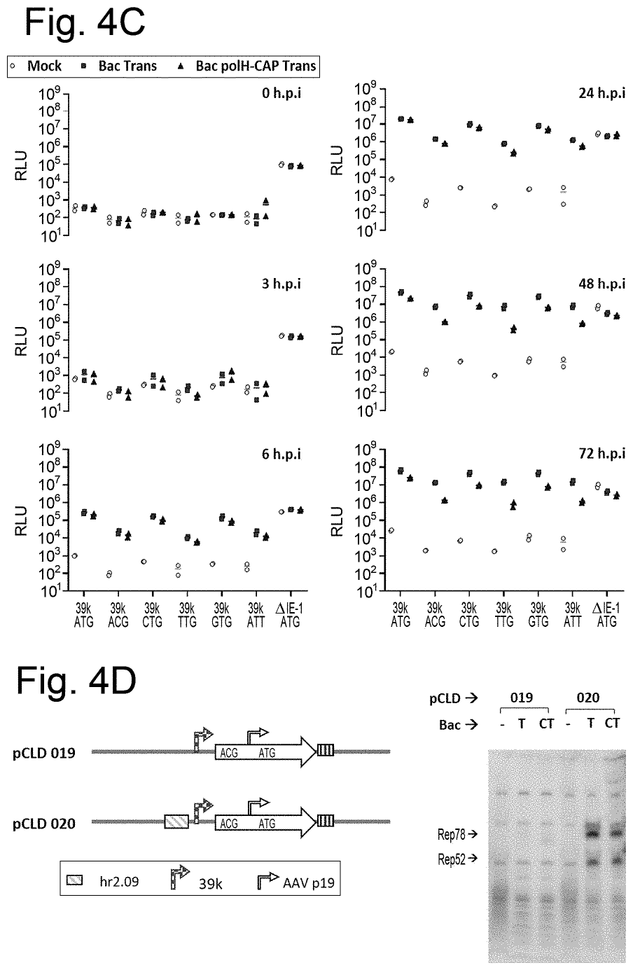

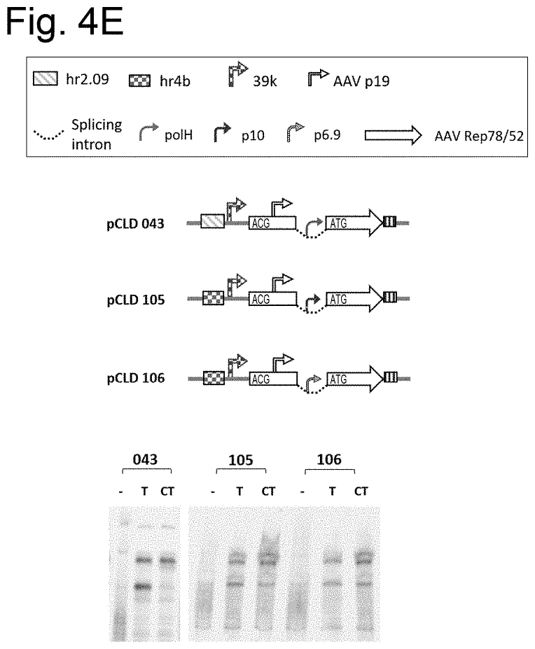

In this study, to overcome the challenge of the constitutive expression profile of ΔIE-1 promoter, the delayed early 39k promoter (SEQ ID NO. 21) (Dong, Z. Q. et al., 2018; Lin, C. H. & Jarvis, D. L., 2013) was used as an alternative for regulating Rep78 expression. The expression profile of 39k promoter was observed to be active as early as 3-6 hours post baculovirus transactivation making it an attractive alternative to be used as a ΔIE-1 temporal mimic (FIG. 4A). Nevertheless, as the expression intensity from 39k promoter regulation was relatively higher as compared to the ΔIE-1, especially at later time points (FIG. 4A), we performed another luciferase reporter assay (FIG. 4B) to screen for alternatives such as using a suboptimal start codon for 39k-regulated gene expression to mimic the ΔIE-1-regulated expression level. As can be seen from FIG. 4C, replacing the ATG start codon with the suboptimal ACG codon would tune down the 39k promoter strength to a level relatively similar to ΔIE-1. Using this suboptimal ACG codon, the indicated pCLDs were designed (FIG. 4D) to test the strength and expression profile of the 39k promoter—ACG combination on full-length AAV2 Rep. Upon the indicated baculovirus transactivation (FIG. 4D), the expression of AAV2 Rep78 could only be detected for pCLD 020 (SEQ ID NO. 09) in which the hr2.09 is still present, indicating that the 39k promoter is still an hr enhancer dependent promoter. However, the expression level of Rep78 was still too high and the ratio between Rep78:Rep52 was far from ideal. The incorporation of relatively strong hr2.09 was suspected to have caused the observed results.

To circumvent this, the expression of the Rep78 was alleviated by changing the enhancer into a relatively weaker, hr4b, while at the same time the Rep52 was enhanced by regulating it with an additional strong late promoter inside of an artificial intron as it has been shown before (Chen, 2008). Several late promoters with the least cis:trans competition with the polH promoter are tested (FIG. 4E). Interestingly, despite the present of a strong hr2.09 enhancer, putting polH as the intronic promoter failed to trigger Rep52 expression when transactivated by the Bac polH Cap Trans. The Rep52 expression could only be restored by replacing the intronic promoter with the p10 or p6.9 (FIG. 4E). This result confirms the present of cis:trans promoter competition among certain Baculovirus promoters and switching promoter could abate this issue. However, the level of Rep52 remains relatively weak as probably caused by the shared use of weaker hr4b enhancer.

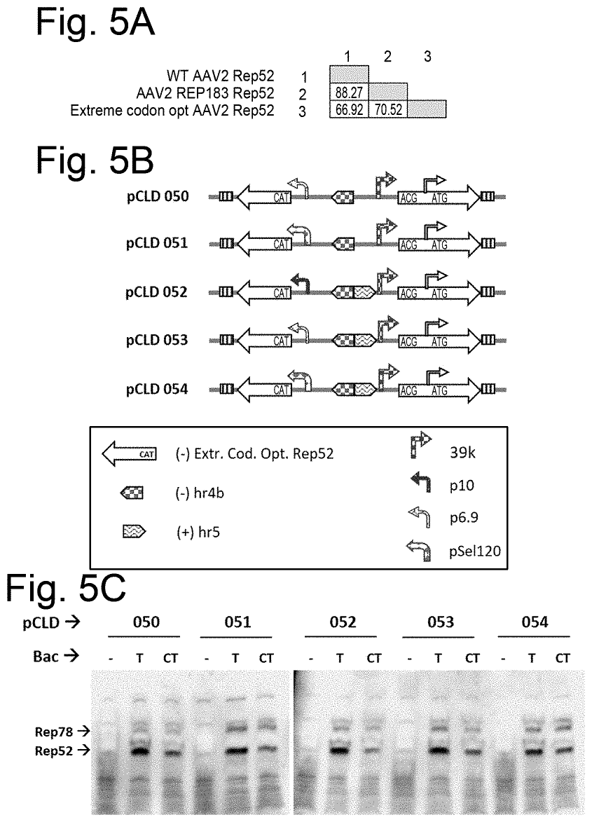

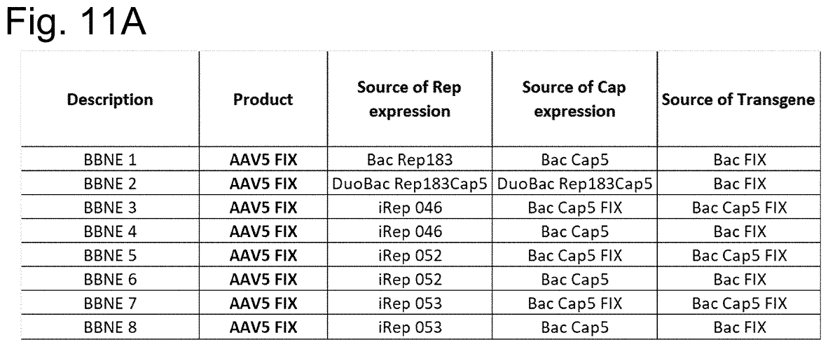

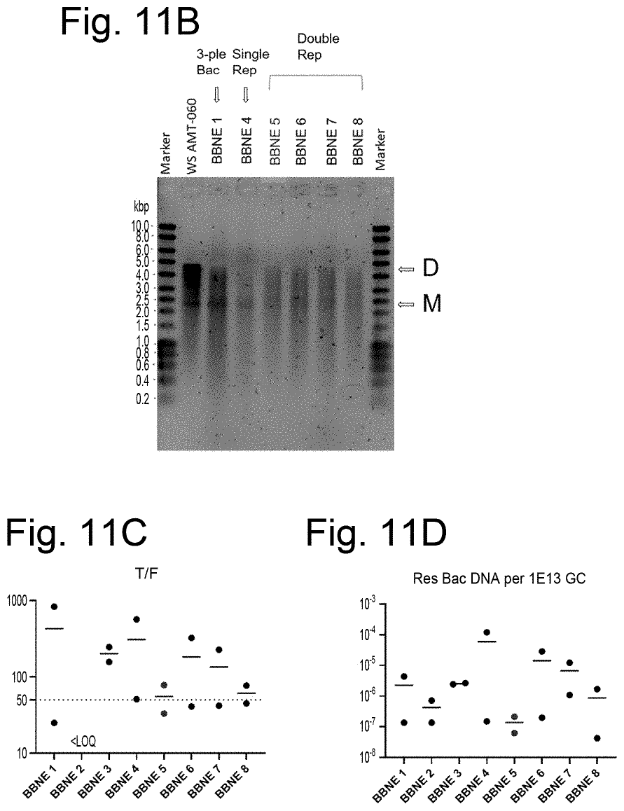

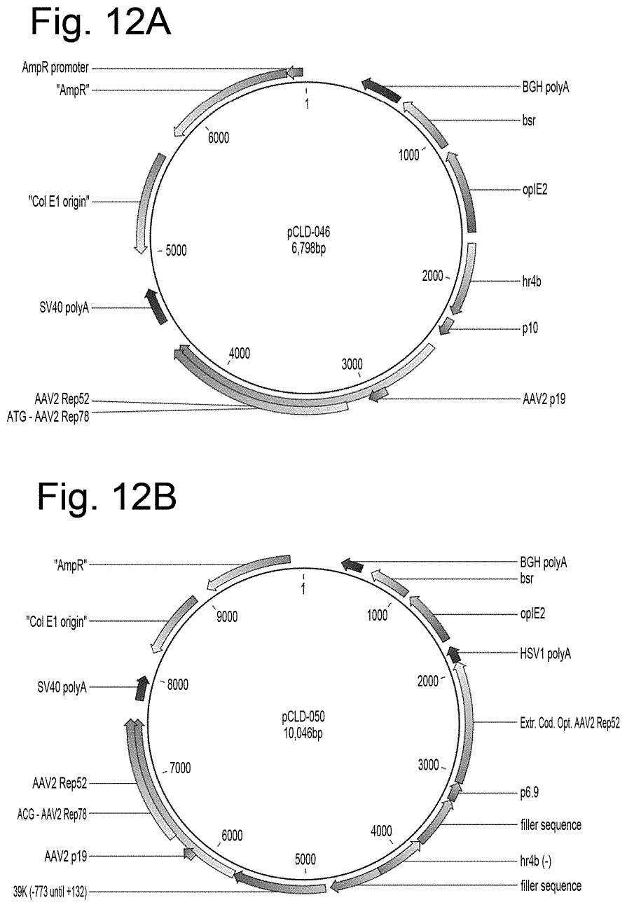

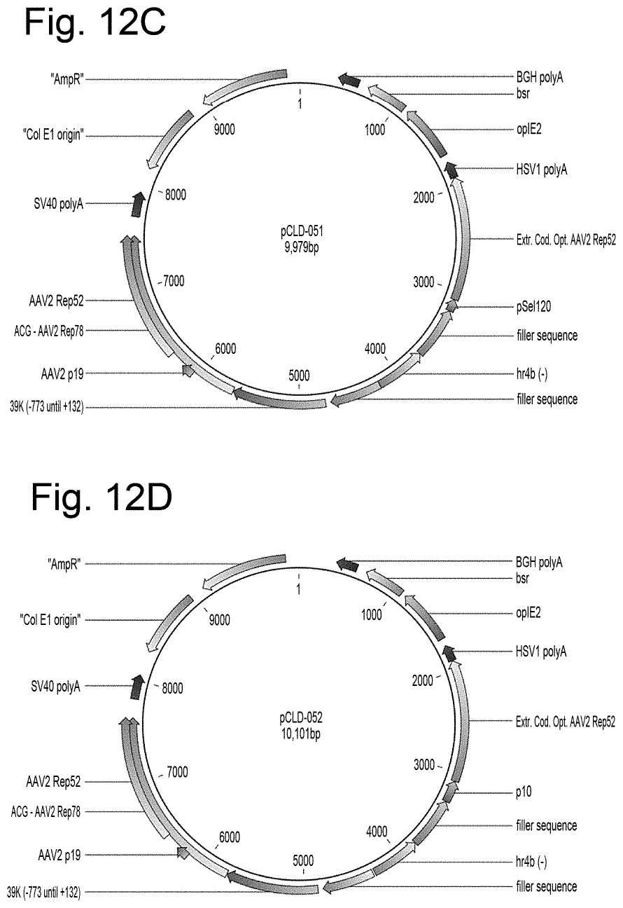

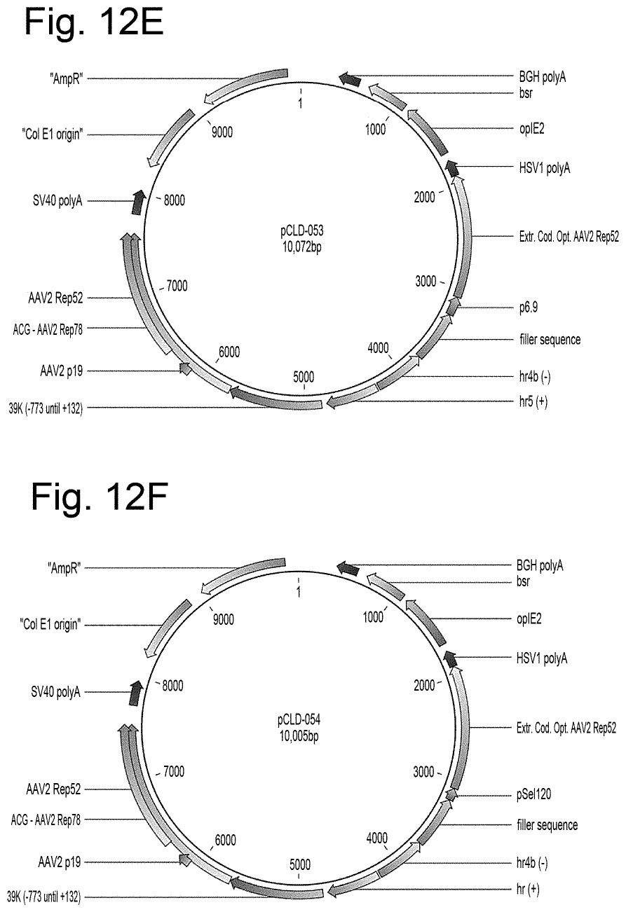

To tackle this, several split-cassette AAV2 Rep constructs (pCLD 050-054, FIG. 12) (SEQ ID NO.s 10-14) were designed and cloned by incorporating the alternative weaker yet non-leaky hr enhancers to further reduce the Rep78 expression. As enhancer activity is known to be bidirectional (FIG. 2C), we tested the capacity of the single hr4b and compared it to the hr4b-hr5 combination (FIG. 5B). Finally, another copy of a codon optimized Rep52 gene (SEQ ID NO. 15) (FIGS. 5A and 11) under the regulation of baculovirus late promoter was added in cis to strengthen the Rep52 expression (FIG. 5B). The expression kinetics and strength of several non-conservative baculovirus late promoters, such as p6.9 (SEQ ID NO. 23), and pSel120 (SEQ ID NO. 24) (Lin, C. H. & Jarvis, D. L., 2013; Martinez-Solis, M., et al., 2016), upon baculovirus transactivation were profiled using the luciferase reporter assay to see their usability as the late promoter regulating the extra copy of Rep52. Interestingly, all these non-conservative baculovirus promoters could be transactivated by the Bac Trans and Bac polH Cap Trans with almost similar potency (FIG. 4A). The maximum difference could only be observed with the Bac polH Cap Trans transactivation of p6.9 promoter at around ±4-fold lower potency at 48 h.p.i., which was still more potent than the earlier p10 promoter (FIGS. 4A and 1C). The transactivation of pSel120 with Bac polH Cap Trans was found to have the highest expression at the very late time point (72 h.p.i). The incorporation of cis:trans-competition-free/-less promoter (p10, p6.9, or pSeI120) would enable several design alternatives of an inducible split-Rep cassette (FIG. 5B). To test these constructs, each of these novel plasmids (pCLD 050-054) were transfected and the AAV2 Rep expression upon the indicated baculovirus transactivation was determined by Western blot (FIG. 5C). As expected, the use of 39k promoter resulted in inducible Rep78 expression regardless of the choice of hr enhancer and/or recombinant baculovirus (FIG. 5C). Although the Rep52 from all these constructs could also be transactivated by any baculovirus, regardless of the late promoters, distinct general expression intensity could be observed between the use of Bac Trans and polH Cap Trans (FIG. 5C), especially for the p10 regulated construct (pCLD 052) which is coherent with the previous results depicted in FIGS. 1B & 3B. The use of alternative late p6.9 and pSeI120 promoters could further ameliorate the cis:trans promoter competition issue upon the Rep52 transactivation by Bac polH Cap Trans. Overall, these results have shown the potential use of alternative baculovirus promoters within inducible AAV split Rep designs for the expression of AAV genes both at the correct timing and intensity. Furthermore, the presented examples offer a possible solution to overcome the cis:trans promoter competition observed during transactivation by recombinant baculovirus harbouring the same promoters as the expression plasmids.

Example 5: The Novel Inducible Split-Rep Cassette in Combination with Single Inoculation of a Baculovirus Harbouring Recombinant polH Promoter can be Used to Produce High Quality AAV Particles

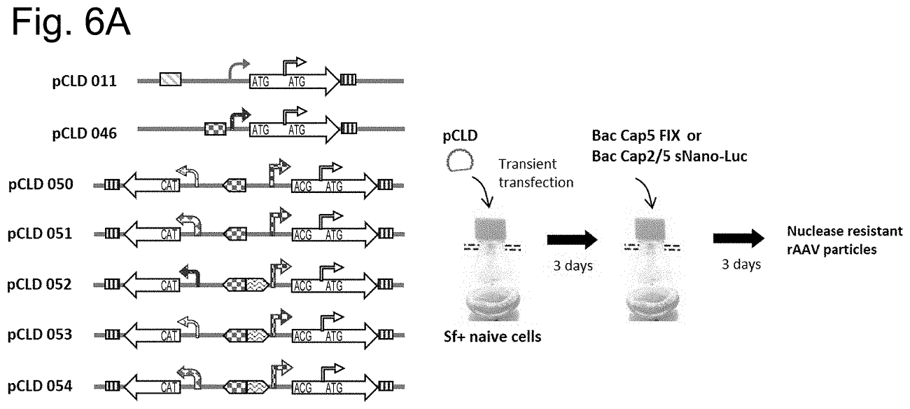

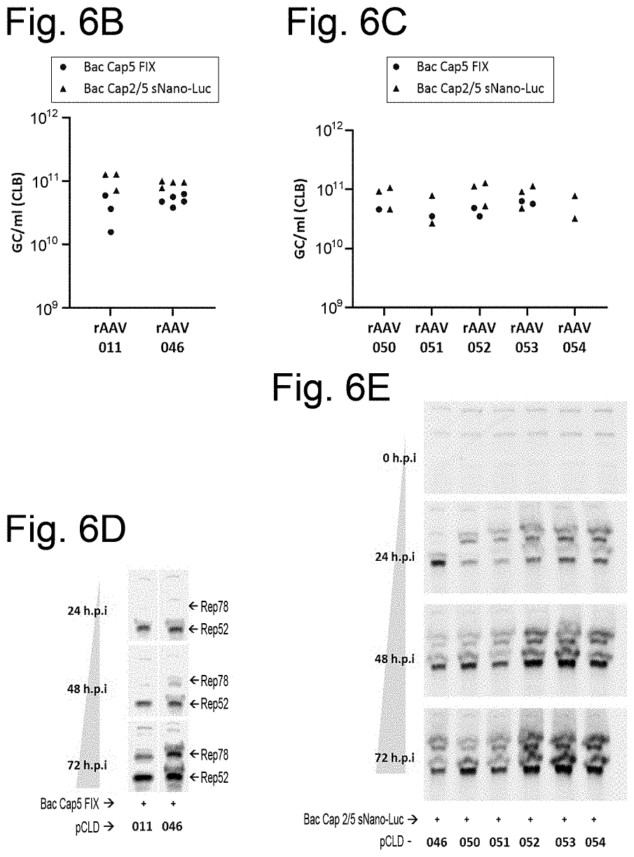

To see if the novel inducible plasmid vectors, pCLD 046 and pCLD 050-054, could be used to produce intact AAV particles, small transient AAV production experiments were performed in ExpresSf+ cells (FIG. 6A). Different Bac polH Cap Trans viruses encoding different AAV Cap serotypes and transgenes were used as transactivating agents (indicated in FIG. 6A). As the benchmark, pCLD 011 was made following Aslanidi et al. (supra) design (FIG. 4A, pCLD 011 (SEQ ID NO. 16). This construct has been reported to be compatible with the Bac Cap Trans design, however the leaky but strong hr2.09 enhancer is still present (Wu, Y., et al., 2019). Overall, the transient AAV production using both the inducible single (pCLD 046) and split-cassette Rep (pCLD 050-054) plasmid vectors could consistently yield a significant output of DNase-resistant AAV particles from several production batches with an average genome copy (GC) titer of ±5×1010 GC/ml in crude lysate buffer (CLB). This titer was on par with the titer of the benchmark construct, pCLD 011 (FIGS. 6B and C). Intriguingly, the expression profile of AAV Rep, especially the temporal expression of Rep78, was different between the benchmark pCLD 011 (FIGS. 6D and E).

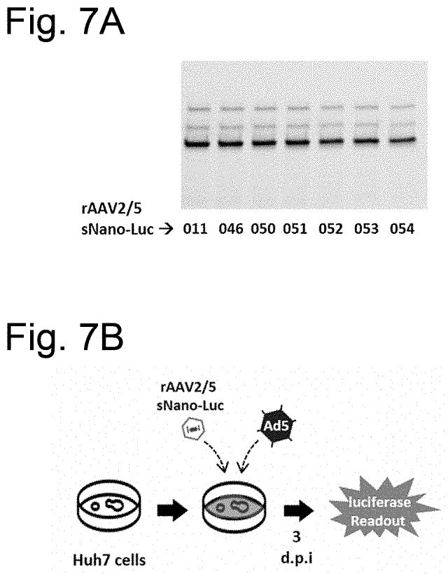

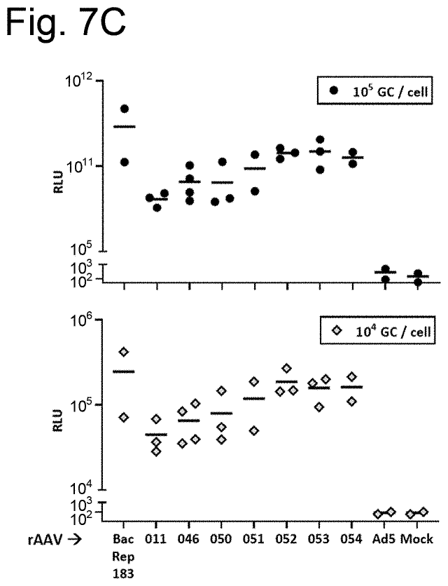

To see the quality parameter of the AAV particles, AVB purification using the material from 40 the small production was performed (FIG. 4D) and analysed. Intriguingly, the capsid ratio (VP1:2:3 ratio) from the purified AAV materials were also comparable to each other (FIG. 7A). Finally, the potency of the AAV particles to transduce target cells (Huh7) was compared against the AAV particles produced using BEV with split-cassette AAV Rep (Bac Rep183) following the depicted protocol (FIG. 7B). From the results, it can be observed that there were potency differences among differently sourced AAV particles (FIG. 7C). Intriguingly, the AAV particles produced from the inducible split-cassette Rep plasmid vectors, especially from the pCLD 052 and 053 constructs, showed higher potency than the single-Rep cassette Rep (pCLD 011 and 046) derived materials. The potency could even reach similar levels with the less robust BEV produced material.

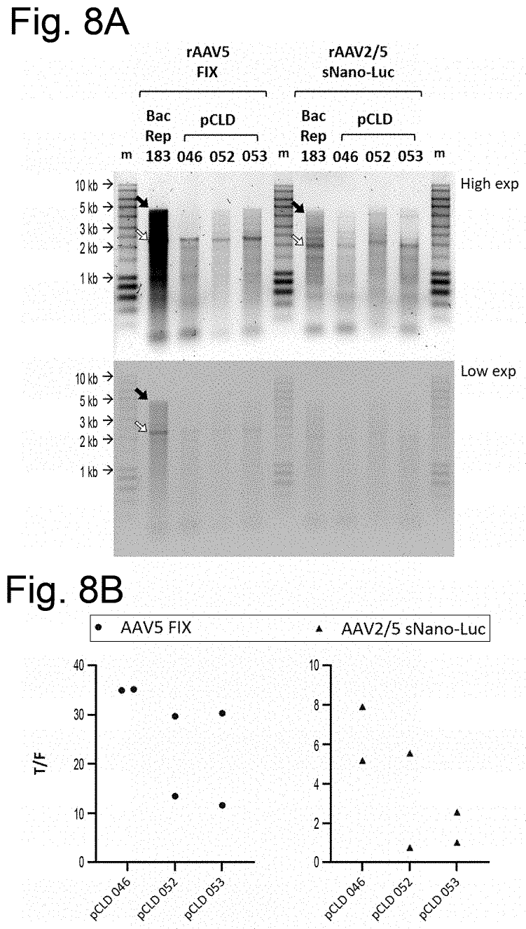

To further study the influence of this novel inducible plasmid vector on AAV particle quality, AAV vector DNA analysis on the AAV particles with the best potency assay results was performed using formaldehyde agarose gel analysis. It is known that BEV derived AAV, particularly produced using the split-Rep cassette, exhibits faster onset and higher potency, probably due to the high packaging rate of multimeric form of the vector DNA (Urabe, M., et al., 2006). This multimeric form would mimic a double-stranded DNA (dsDNA) form circumventing the rate-limiting single-stranded (ssDNA) to dsDNA formation prior to gene expression (McCarty, D. M., 2008). In this study, the expected size of AAVS FIX- and AAV2/5 nano-luciferase vector genomes are 2.5 kb and 2 kb respectively. The majority of pCLD 046 or single-cassette-Rep produced AAV vector genomes are single-stranded monomer as could be seen from the FIG. 8A. However, in addition to the 2 or 2.5-kb single-stranded vector genome, DNA extracted from AAV particles produced using pCLD 052 and 053 contained additional multimeric genomes with cut-off size exactly at the maximum AAV packaging capacity of 4.7 kb (FIG. 8A). These results further prove that the pCLD 052 and 053 could exactly mimic the performance of BEV with split-cassette Rep in packaging the multimeric vector genomes, which correlates to the in vitro potency assay results. Finally, to prove the superiority of split Rep-cassette over the single-Rep cassette, we performed a head to head hypothetical T/F comparison on purified AAV material produced using the indicated pCLD (FIG. 8B). Here, we could see that the split-Rep cassette design indeed has a tendency to produce AAV with lower T/F value, indicating higher content of full particles and the superiority of the design (pCLD 052 and 053).

In general, the combination of an alternative and non-leaky hr enhancer together with alternative baculovirus promoter with less cis:trans competition (39k, p10, p6.9, and pSe1120) can be implemented to generate novel inducible split-Rep cassette plasmid vectors that can be transactivated by Bac polH Cap Trans. These vectors, especially the pCLD 052 and 053, are very useful to generate next generation stable packaging insect cell lines.

### Example 6: The Generation of Novel Stable rAAV packaging Cells

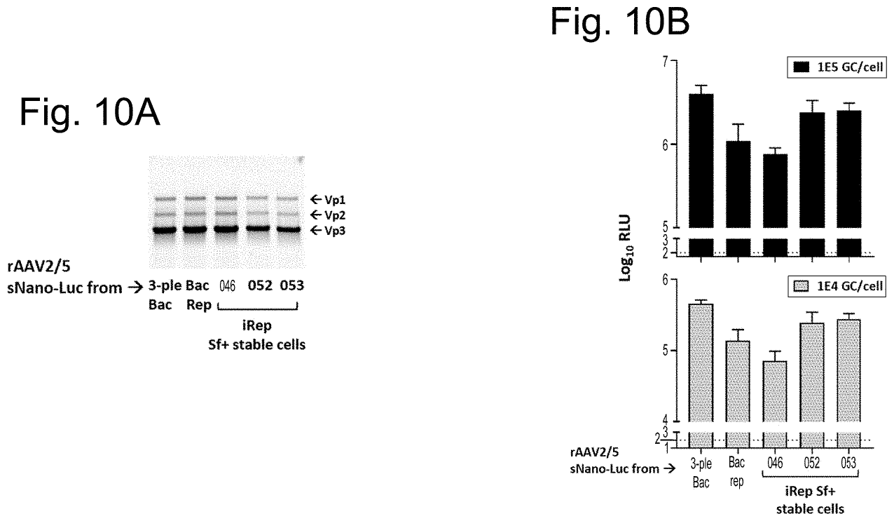

To see if we can generate stable cell-lines/pools that would require only a single Baculovirus inoculation for producing AAV, we performed stable cell-line generation with the selected inducible AAV-Rep plasmid used in the transient transfection study (pCLD 046, 052, and 053) as could be 40 seen as detailed steps in the Materials and Methods section or in a nutshell in FIG. 9A. The generated novel cell-line would next be called as insect inducible Rep cell-lines or iRep cell-lines (iRep 046, iRep 052, and iRep 053). To investigate whether AAV could be produced from the stable iRep cell-lines, the cells were expanded as usual, similar with the wild-type ExpresSf+ cells, and infected by Bac polH Cap Trans (Bac Cap5 FIX or Bac Cap2/5 sNano-Luc) and the CLB was harvested to measure the DNase-resistant AAV particle GC concentration. As could be observed, the iRep cell-lines, regardless of the inducible Rep design, could yield a relatively abundant AAV particles reaching ±1×10¹¹ GC/ml titer or higher than 1×10⁵ GC productivity per cell depending on the type of the AAV (FIG. 9B). Interesting the AAV Rep expression profile from the stable iRep cell-lines was also in line with the profile seen in the transient transfection results (FIGS. 6E and 9C). To further check on the AAV particle functionality and quality, AVB purification was performed and analyzed. Intriguingly, similar with the transient transfection result, the capsid ratio (VP1:2:3 ratio) from the purified representative AAV2/5 materials were also comparable to each other (FIG. 10A). Finally, the potency of the AAV particles to transduce target cells (Huh7) were compared against the AAV particles produced using BEV with split-cassette AAV Rep (Bac Rep183) following the depicted protocol (FIG. 7B). From the results, it can be observed that there were potency differences among differently sourced AAV particles (FIG. 10B) with similar bias confirming the transient transfection results (FIG. 7C).

To further analyze the particle quality, the AVB purified materials (BBNE) produced from the novel iRep cell-lines were compared to other methods, including the duo or dual bac inoculation method (FIG. 11A). Similar with the transient transfection outcome (FIG. 8A), the monomeric-dimeric pattern of the packaged AAV DNA could only be seen in the materials produced from the iRep 052 and 053 cell-line but not the iRep 046, regardless of the bac inoculation approach (FIG. 11B). However, further results from the hypothetical T/F and baculovirus genomic DNA contamination analysis showed that only the iRep 052 cell-line with single inoculation of bac harboring the capsid and transgene could produce AAV particles with relatively excellent quality in the most consistent manner (FIG. 11C and D).

### Example 7: Sequential Batches Reactors (SBR) Study Using the Selected iRep 052 Cell Lines

As an intermediate step towards the generation of a new production cell line with integrated Rep genes, it was necessary to generate a polyclonal culture of iRep Express SF+ by transfection of the parental cell line with a DNA plasmid pCLD-052, which carried a AAV Rep cassette. In order to evaluate the stability and the expression of the integrated Rep genes in this stable cell pool, we expanded the cell culture in sequential batches reactors (SBR) and checked the Rep genes expression at different cell passages in 1 L shake flask. SBR is a repetitive batches process where filling and withdrawal take place sequentially in a bioreactor. We used the SBR system over manual daily transfer in shake flask to allow cultivation condition standardization (e.g. oxygen supply) which gives a better reproducibility and more consistent results, and to mimic the conditions that the cells will experience under production conditions.

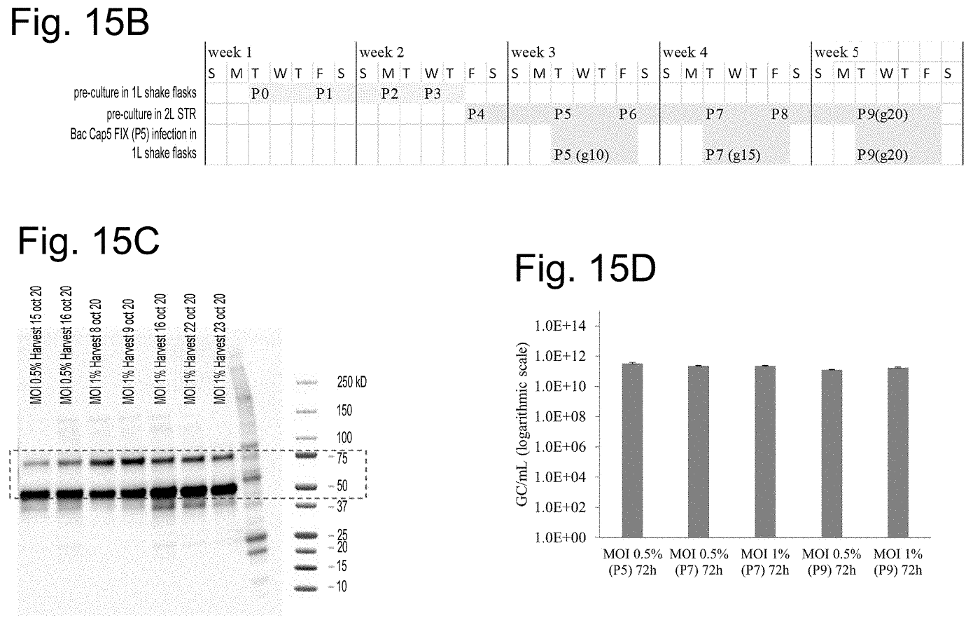

We first grew the stable cell pool in 1 L shake flasks (FIG. 15A) from cell thaw (P0) to cell passage 3 (P3) and then passaged the cell pool from cell passage 4 (P4) to passage 9 (P9) in a 2 L bioreactor via SBR system (FIG. 15A). The seed train production took in total 5 weeks (FIG. 15B). We used the stable pool at passages 5, 7, and 9 for AAV production by transfection with baculovirus Bac Cap5 FIX, which harbors an expression cassette for AAV Cap genes and a Factor IX (FIX) transgene flank by AAV-ITRrs (FIG. 15A). The stable pool at passage 5, 7, and 9 are equivalent to the seed train production in 500L, 2000L, and above 10000L process. The purpose of this document is to describe a 2L process to validate the expression and the stability of the integrated Rep genes in the iRep Express Sf+ stable cell pool, wherein we also measured the genome copies of the transgene from the filtered crude lysed bulk (FCLB) at 72 hours post-transfection with Bac Cap5 FIX.

In order to validate the stability of the iRep stable pool which is a polyclonal culture, we checked the expression of the integrated Rep genes of the culture at passage 5, 7, and 9 by Western blot (FIG. 15C). Since the expression of the integrated Rep genes (Rep78 and Rep52) in the iRep stable pool is regulated by the hr2.09 and the promoter of the baculovirus Bac Cap5 FIX, we transfected the iRep stable pool culture at passage 5, 7 and 9 separately in 1L shake flasks with baculovirus Bac Cap5 FIX (passage 5) to activate the expression of the integrated Rep genes in the iRep stable pool. Cell lysate samples were taken from the shake flasks at 48 and 72 hours of post-transfection with baculovirus Bac Cap5 FIX (P5). As shown in FIG. 15, we confirmed the expression of Rep78 and Rep52 in the iRep stable pool cell at cell passage 5, 7 and 9, wherein distinct protein products Rep78 and Rep 52 were observed from each protein extract sample of the cell lysates (dashed box, FIG. 15C). After 20 generations (passage 9), the expression of the integrated Rep genes remained stable. From all the protein extracts, the intensity of the immunosignal from the protein Rep52 was higher than the protein Rep 72. This expression ratio of Rep78 and Rep52 is commensurate with a normal AAV2 Rep expression (low Rep78 and high Rep52).

To further confirm the AAV production of the iRep stable pool with single baculovirus transfection (UnoBac platform), We also measured the genome copies (GC) of Factor IX (FIX) in the FCLB from the transfection of baculovirus Bac Cap5 FIX (P5) with the iRep stable pool cell at passage 5, 7 and 9 (FIG. 15D). FIX has been used in the replacement therapy of hemophilia B and was harbored in the recombinant Cap-Trans-cassette of baculovirus Bac Cap5 FIX. The expression of the integrated Rep78 and Rep52 in the iRep stable pool was transactivated by the early and late polH promoters of the Cap-Trans cassette in Bac Cap5 FIX, respectively. With the cascade expression of the Cap and transgenes, in this case FIX, during transfection, iRep stable pool generated AAV2 encapsulated FIX. As shown in FIG. 15D, the average GC titer in all FCLB samples were all above 1e11 GC/mL. The GC titer (FIX) at different passages remained steady, and this confirms the stability of the integrated Rep genes cassette in the iRep stable pool. This also confers the potential of using iRep cell line for up-scaling production of AAV by UnoBac system.

### REFERENCES

Burnett, J. R. & Hooper, A. J. Alipogene tiparvovec, an adeno-associated virus encoding the Ser(447)X variant of the human lipoprotein lipase gene for the treatment of patients with lipoprotein lipase deficiency. Curr Opin Mol Ther 11, 681-691 (2009).

Yla-Herttuala, S. Endgame: glybera finally recommended for approval as the first gene therapy drug in the European union. Mol Ther 20, 1831-1832, doi:10.1038/mt.2012.194 (2012).

Cheng, X. H., Hillman, C. C., Zhang, C. X. & Cheng, X. W. Reduction of polyhedrin mRNA and protein expression levels in Sf9 and Hi5 cell lines, but not in Sf21 cells, infected with Autographa californica multiple nucleopolyhedrovirus fp25k mutants. J Gen Virol 94, 166-176, doi:10.1099/vir.0.045583-0 (2013).

Garretson, T. A., Shang, H., Schulz, A. K., Donohue, B. V. & Cheng, X. W. Expression- and genomic-level changes during passage of four baculoviruses derived from bacmids in permissive insect cell lines. Virus Res 256, 117-124, doi:10.1016/j.virusres.2018.08.009 (2018).

Aslanidi, G., Lamb, K. & Zolotukhin, S. An inducible system for highly efficient production of recombinant adeno-associated virus (rAAV) vectors in insect Sf9 cells. Proc Natl Acad Sci USA 106, 5059-5064, doi:10.1073/pnas.0810614106 (2009).

Mietzsch, M. et al. OneBac: platform for scalable and high-titer production of adeno-associated virus serotype 1-12 vectors for gene therapy. Hum Gene Ther 25, 212-222, doi:10.1089/hum.2013.184 (2014).

Mietzsch, M. et al. OneBac 2.0: Sf9 Cell Lines for Production of AAV1, AAV2, and AAV8 Vectors with Minimal Encapsidation of Foreign DNA. Hum Gene Ther Methods 28, 15-22, doi:10.1089/hgtb.2016.164 (2017).

Mietzsch, M., Casteleyn, V., Weger, S., Zolotukhin, S. & Heilbronn, R. OneBac 2.0: Sf9 Cell Lines for Production of AAVS Vectors with Enhanced Infectivity and Minimal Encapsidation of Foreign DNA. Hum Gene Ther 26, 688-697, doi:10.1089/hum.2015.050 (2015).

Wu, Y. et al. Development of Versatile and Flexible Sf9 Packaging Cell Line-Dependent OneBac System for Large-Scale Recombinant Adeno-Associated Virus Production. Hum Gene Ther Methods 30, 172-183, doi:10.1089/hgtb.2019.123 (2019).

van Oers, M. M., Pijlman, G. P. & Vlak, J. M. Thirty years of baculovirus-insect cell protein expression: from dark horse to mainstream technology. J Gen Virol 96, 6-23, doi:10.1099/vir.0.067108-0 (2015).

Ghosh, S., Jain, A., Mukherjee, B., Habib, S. & Hasnain, S. E. The host factor polyhedrin promoter binding protein (PPBP) is involved in transcription from the baculovirus polyhedrin gene promoter. J Virol 72, 7484-7493 (1998).

Dong, Z. Q. et al. Construction and characterization of a synthetic Baculovirus-inducible 39K promoter. J Biol Eng 12, 30, doi:10.1186/s13036-018-0121-8 (2018).

Lin, C. H. & Jarvis, D. L. Utility of temporally distinct baculovirus promoters for constitutive and baculovirus-inducible transgene expression in transformed insect cells. J Biotechnol 165, 11-17, doi:10.1016/j.jbiotec.2013.02.007 (2013).

Martinez-Solis, M., Gomez-Sebastian, S., Escribano, J. M., Jakubowska, A. K. & Herrero, S. A novel baculovirus-derived promoter with high activity in the baculovirus expression system. PeerJ 4, e2183, doi:10.7717/peerj.2183 (2016).

Urabe, M. et al. Scalable generation of high-titer recombinant adeno-associated virus type 5 in insect cells. J Virol 80, 1874-1885, doi:10.1128/JVI.80.4.1874-1885.2006 (2006).

Urabe, M., Ding, C. & Kotin, R. M. Insect cells as a factory to produce adeno-associated virus type 2 vectors. Hum Gene Ther 13, 1935-1943, doi:10.1089/10430340260355347 (2002).

Hermens, W. T. J. M. C., Haast, S.J.P., Biesmans ,D.J., et al. Vectors with modified initiation codon for the translation of AAV-Rep78 useful for production of AAV. W02009/014445 (2009).

Bleckmann, M. et al. Identification of Essential Genetic Baculoviral Elements for Recombinant Protein Expression by Transactivation in Sf21 Insect Cells. PLoS One 11, e0149424, doi:10.1371/journal.pone.0149424 (2016).

Rodems, S. M. & Friesen, P. D. The hr5 transcriptional enhancer stimulates early expression from the Autographa californica nuclear polyhedrosis virus genome but is not required for virus 15 replication. J Virol 67, 5776-5785 (1993).

Venkaiah, B., Viswanathan, P., Habib, S. & Hasnain, S. E. An additional copy of the homologous region (hr1) sequence in the Autographa californica multinucleocapsid polyhedrosis virus genome promotes hyperexpression of foreign genes. Biochemistry 43, 8143-8151, doi:10.1021/bi049953q (2004).

Olson, V. A., Wetter, J. A. & Friesen, P. D. The highly conserved basic domain I of baculovirus 1E1 is required for hr enhancer DNA binding and hr-dependent transactivation. J Viro177, 5668-5677, doi:10.1128/jvi.77.10.5668-5677.2003 (2003).

Hermens, W. T. J. M. C., et al. BACULOVIRAL VECTORS COMPRISING REPEATED CODING SEQUENCES WITH DIFFERENTIAL CODON BIASES. W02009/014445 (2009).

McCarty, D. M. Self-complementary AAV vectors; advances and applications. Mol Ther 16, 1648-1656, doi:10.1038/mt.2008.171 (2008).

Chen, H. Intron splicing-mediated expression of AAV Rep and Cap genes and production of AAV vectors in insect cells. Mol Ther 16, 924-930, doi:10.1038/mt.2008.35 (2008).

Wang, Y., Wang, F., Wang, R., Zhao, P. & Xia, Q. 2A self-cleaving peptide-based multi-gene 30 expression system in the silkworm Bombyx mori. Sci Rep 5, 16273, doi:10.1038/srep16273 (2015).

1. An insect cell comprising, integrated into the genome of the cell:
(i) a first promoter operably linked to a nucleotide sequence encoding an mRNA, translation of which in the cell produces at least one of parvoviral Rep 78 and 68 proteins;
(ii) a second promoter operably linked to a nucleotide sequence encoding an mRNA, translation of which in the cell produces at least one of parvoviral Rep 52 and 40 proteins; and
(iii) at least one enhancer element that is operably linked to the first and second promoters, wherein the at least one enhancer element is dependent on a transcriptional transregulator, wherein introduction of the transcriptional transregulator into the cell induces transcription from the first and second promoters.

2. The insect cell according to claim 1, wherein the first and second promoters are baculoviral promoters, the transcriptional transregulator is a baculoviral immediate-early protein (IE1) or its spice variant (1E0) and the transcriptional transregulator-dependent enhancer element is a baculoviral homologous region (hr) enhancer element.

3. The insect cell according to claim 1, wherein the baculovirus is Autographa californica multicapsid nucleopolyhedrovirus.

4. The insect cell according to claim 2, wherein the hr enhancer element is a hr enhancer element other than hr2-0.9, and comprises at least one copy of the hr 28-mer sequence CTTTACGAGTAGAATTCTACGCGTAAAA and/or at least one copy of a sequence of which at least 20 nucleotides are identical to sequence CTTTACGAGTAGAATTCTACGCGTAAAA and which binds to a baculoviral IE1 protein, and wherein the hr enhancer element, when operably linked to an expression cassette comprising a reporter gene operably linked to the polH promoter,
a) under non-inducing conditions, the expression cassette with the hr enhancer element produces less reporter transcript than an otherwise identical expression cassette which comprises the hr2-0.9 element, or the cassette with the hr enhancer element produces less than a factor 1.1, 1.2, 1.5, 2, 5 or 10 of the amount reporter transcript produced by an otherwise identical expression cassette which comprises the hr4b element; and,
b) under inducing conditions, the expression cassette with the hr enhancer element produces at least 50, 60, 70, 80, 90 or 100% of the amount of reporter transcript produced by an otherwise identical expression cassette which comprises the hr4b or the hr2-0.9 element.

5. The insect cell according to claim 4, wherein the hr enhancer element is selected from the group consisting of hr1, hr3, hr4b and hr5, of which hr4b and hr5.

6. The insect cell according to claim 2, wherein the first and second promoters are distinct, the first promoter is a delayed early baculoviral promoter and the second promoter is a late or very late baculovirus promoter.

7. The insect cell according to claim 6, wherein the first promoter is the 39k promoter and the second promoter is selected from the group consisting of the polH, p10, p6.9 and pSel120 promoters.

8. The insect cell according to claim 1, wherein at least one of the parvoviral Rep 52 and 40 proteins and at least one of parvoviral Rep 78 and 68 proteins have a common amino acid sequence that is at least 90% identical, while the nucleotide sequence encoding the common amino acid sequence in the mRNA for the at least one of parvoviral Rep 52 and 40 proteins has less than 60% sequence identity with the nucleotide sequence encoding the common the amino acid sequence in the mRNA for the at least one of parvoviral Rep 78 and 68 proteins.

9. The insect cell according to claim 8, wherein the codon usage in the nucleotide sequence encoding the common the amino acid sequence in the mRNA for at least one of the parvoviral Rep 52 and 40 proteins, is more adapted to the codon usage bias of the insect cell than codon usage in the nucleotide sequence encoding the common the amino acid sequence in the mRNA for at least one of the parvoviral Rep 78 and 68 proteins.

10. The insect cell according to claim 1, wherein the nucleotide sequence encoding the mRNA for the at least one of parvoviral Rep 78 and 68 proteins comprises a modification that affects a reduced steady state level of the at least one of parvoviral Rep 78 and 68 proteins comprising an open reading frame that starts with a suboptimal translation initiation codon selected from ACG, CTG, TTG, GTG and ATT, of which ACG is most preferred.

11. The insect cell according to claim 1, wherein the first and second promoters are integrated in the cell's genome in opposite directions of transcription and wherein the at least one enhancer element is present in between the first and second promoters, wherein the two enhancer elements are optionally present in between the first and second promoters.

12. The insect cell according to claim 1, wherein the cell further comprises:
(a) a nucleotide sequence comprising parvoviral capsid protein coding sequences operably linked to a third promoter for expression in the insect cell;
(b) a nucleotide sequence comprising a transgene that is flanked by at least one parvoviral inverted terminal repeat sequence; and,
(c) a nucleotide sequence comprising an expression cassette for expression of the transcriptional transregulator.

13. The insect cell according to claim 12, wherein the nucleotide sequences of at least one of (a), (b) and (c) are comprised in the baculoviral vector comprising the expression cassette for expression of the transcriptional transregulator.

14. The insect cell according to claim 8, wherein the first promoter is active before the third promoter.

15. The insect cell according to claim 1, wherein the at least one of parvoviral Rep 78 and 68 proteins, the at least one of parvoviral Rep 52 and 40 proteins, the parvoviral VP1, VP2, and VP3 capsid proteins and the at least one parvoviral inverted terminal repeat sequence are from an adeno associated virus (AAV).

16. The insect cell according to claim 1, comprising cap-coding sequences selected from CAP AAV2/5 (SEQ ID NO. 29) and AAVS (SEQ ID NO. 30).

17. A method for producing a recombinant parvoviral virion, comprising:
(a) culturing an insect cell according to claim 1;
(b) providing the cell with:
(i) a nucleotide sequence comprising parvoviral capsid protein coding sequences operably linked to a third promoter for expression in the insect cell;
(ii) a nucleotide sequence comprising a transgene that is flanked by at least one parvoviral inverted terminal repeat sequence; and,
(iii) a nucleotide sequence comprising an expression cassette for expression of the transcriptional transregulator, and
(c) recovering the recombinant parvoviral virion.

18. The method according to claim 17, wherein recovery of the recombinant parvoviral virion comprises at least one of affinity-purification of the virion using an immobilised anti-parvoviral antibody, and filtration over a filter having a nominal pore size of 30-70 nm.

19. The method according to claim 18, wherein the antibody is a single chain camelid antibody or a fragment thereof.

20. A kit of parts comprising at least an insect cell according to claim 1 and a baculoviral vector and/or
(i) a nucleotide sequence comprising parvoviral capsid protein coding sequences operably linked to a third promoter for expression in the insect cell;
(ii) a nucleotide sequence comprising a transgene that is flanked by at least one parvoviral inverted terminal repeat sequence; and,
(iii) a nucleotide sequence comprising an expression cassette for expression of the transcriptional transregulator.


SEQUENCE LISTING (55 sequences)

SEQ ID NO: 1 - DNA, 12538 bp, synthetic construct
  misc_feature 1..12538: note = Bac Trans
  source 1..12538: mol_type = other DNA
cgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcagggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgataatacgactcactatagggagacccaagcttgatcccccctcttcctcctcctcaagggaaagctgcccacttctagctgccctgccatcccctttaaagggcgacttgctcagcgccaaaccgcggctccagccctctccagcctccggctcagccggctcatcagtcggtcaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttgtccgtgttgcttggtcttcacctgtgcagaattgcgaaccatggattcatcgacggtaccgcgggccctcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccccctgcagcctgcattaatgaatcggccaacgcgcggggagaggcggtttgcgtattgggcgctcttccgcttcctcgctcactgactcgctgcgctcggtcgttcggctgcggcgaggggggtaccagatcccatgggagctctgcagaattctctagaggcctcgcgagatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgat

SEQ ID NO: 2 - DNA, 11544 bp, synthetic construct
  misc_feature 1..11544: note = Bac polH Cap2/5
  source 1..11544: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatctgagctcggcgcgtgtaatgagacgcacaaactaatatcacaaactggaaatgtctatcaatatatagttgctgatgtaccgcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttgccgccaccctggctgccgatggttatctacccgattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggccgccccgggggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 3 - DNA, 14299 bp, synthetic construct
  misc_feature 1..14299: note = Bac polH Cap5 - human Factor IX
  source 1..14299: mol_type = other DNA
tgtaatgagacgcacaaactaatatcacaaactggaaatgtctatcaatatatagttgctgatgtaccgcagcatgctatgcatcagctgctagtactccggaatattaatagatcatggagataattaaaatgataaccatctcgcaaataaataagtattttactgttttcgtaacagttttgtaataaaaaaacctataaatagaccggagtagtcataccgtcccaccatcgggcgcggatcgtaccgggcccaagcttcctgttaagacggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctagagcctgcagtctcgacaagctagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggggggtaccagatcccatgggagctctgcagaattctctagaggcctcgcgagatcgatctagaaagcttcccggggggatctgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgctgagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctgactcgatgctttatttgtgaaatttgtgatgctattgctttatttgtaaccattataagctgcaataaacaagttaacaacaacaattgcattcattttatgtttcaggttcagggggaggtgtgggaggttttttaaactaggtcacgactccacccctccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccagatccccccgggaagctttctagatcgatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccat

SEQ ID NO: 4 - DNA, 13365 bp, synthetic construct
  misc_feature 1..13365: note = Bac Rep183
  source 1..13365: mol_type = other DNA
accgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagtcgagattaccctgttatccctaccagtgtgttggatttattgttcaaagatacagtcatccaaatccacattaaccagatcgcaggcagtacaagcgtctggcacttttcccatgatatgatgaatatagcataatttttgatacgccttttttacgacagaaacgggttgagattctgacaccggaaagcattctaaacagtctttctggccgtgagtgaaacagatattactattctgattcattctctcacattgtctgcagggaaacaacattaagttcatgcctacgtgacgagaacatttgttttggtagcggtctgcgtagtttatcgaagcttccgcatctgacgtgcttggctgcgcaaccgattctctcactcgtttgggctcacttatatctgcatcactcggggcgggtcttttcttagcaccaccttttttgacgtaaaattcatgttccacttcaacaacgtgatcctttgcccaacgaaagaagtctttgacttcttgttttgttaccttgccaaaatcatgatccagtcggcgcgtcaattcaaatttgaacattcggtcttgcaacggttgttggtgttcgaatgtcgtactgttaccgtcaatcacggcgcacatgttcgtgttgcttgtaacgatcaccggtgtcgggtctatctgcgcagagcttttgcatttctggtctacgcgcactttgctgcctcctaaaattgctttggccgactccacgactttagcggtcattttgccttcctcccaccaaataaccatcttgtcgacacagtcgttgaatggaaagttctcattggtccagttaacgcagccataaaaaggtacagtgtgggctatggcctccgctatgtttgtttttcccgtagttgcaggtccaaacaaccaaatggtgtttcttttgccaaactttttcgtcgcccagcccaaaaatacggaagccgcatattgaggatcgtagccgtttaactccaaaatcttatagatgcgattgctggaaatgtcttccacgggttgctggcccaccaggtagtcgggggcggttttagtcaggctcataatcttgcccgcattgtccaaggcagctttgatttggctacgcgagttggatgccgcattaaacgagatgtatgaggcttgatcttcttgtatccattgcttctccgaggtaatacccttgtccaccaaccaaccgaccaattccatggcgaccgagatccgcgcccgatggtgggacggtatgaataatccggaatatttataggtttttttattacaaaactgttacgaaaacagtaaaatacttatttatttgcgagatggttatcattttaattatctccatgatctattaatattccggagtactgctagcaccatggatcccggtccgaagcgcgcggaattcaaaggcctacgtcgacgagctcactagtcgcggccgatctaataaacgataacgccgggtggcgtgaggcatgtaaaaggttacatcattatcttgttcgccatccggttggtataaatagacgttcatgttggtttttgtttcagttgcaagttggctgcggcgcgcgcagcacctttgcggccatctgcagaattcgcccttgttactcttcagccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaggcctcgcgagatcttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagt

SEQ ID NO: 5 - DNA, 7311 bp, synthetic construct
  misc_feature 1..7311: note = pCLD 002
  source 1..7311: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttggccgcgaattcactagtgattgcggaataattgccatatgtaaatgatgtcatcgttctaactcgctttacgagtagaattctacgtgtaaaacataatcaagagatgatgtcatttgtttttcaaaactgaactcaagaaatgatgtcatttgtttttcaaaactgaactggctttacgagcagaattctacttgtaacgcatgatcaagggatgatgtcatttgttttttaaaattgaactggctttacgagtagaattctacttgtaaaacacaatcgagagatgatgtcatattttgcacacggctctaattaaactcgctttacgagtaaaattctacttgtaacgcatgatcaagggatgatgtcattggatgagtcatttgtttttcaaaactaaactcgctttacgagtagaattctacttgtaaaacacaatcaagggatgatgtcattatacaaatgatgtcatttgtttttcaaaactaaactcgctttacgggtagaattctacttgtaaaacacaatcgagggatgatgtcatcctttacacatgattataaacgtgtttatgtatgactcatttgtttttcaaaactaaactcgctttacgagtagaattctacttgtaacgcacgatcaagggatgatgtcatttatttgtgcaaagctgatgtcatcttttgcacacgattataaacacaatcaaataatgactcatttgttttcaaaactgaactcgctttacgagtagaattctacttgtaaaacacaatcaagggatgatgtcattttaaaaatgatgtcatttgtttttcaaaactaaactcgctttacgagtagaattctacgtgtaaaacacaatcaagggatgatgtcatttactaaaataaaataattatttaaataaaaatgtttttattgtaaaatacacattgattacacgtgacaatcgaattcccgcttgctagcttcttaagttagatctttatgcatttcggagcgagaccatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtaacggccgccagtgtgctggaattcgcccttcgcggatcctgttaagacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 6 - DNA, 7640 bp, synthetic construct
  misc_feature 1..7640: note = pCLD 046
  source 1..7640: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtctcaggcttaattaatgatgcgatcgccgttacacgtttatgcgggccgaggcacatgcggcaggatgacggtccataccagattcctgggcacaactcatcggcaacgttccatcgtcctgctcctccacctcgggcgggaagccatggctcagttttttatagagctcatccattccgagtgttatacctgccgctgtcacaaattcaagcagcaccatgtggtcgcgtttctcgttcggatcctttgacagttttgattggtatgacaaatagtgattatctgggagcaggactggtccatctcctataggggtgttttgttgataatgatcggcaagttgcacgctaccgtcctctatgttgtggcgaatcttgaaatttacctttataccgtttttctgcttgtctgccataatatagacgttgtgcgagttataattgtattccaacttgtgtcccaatatgtttccatcttctttgaagtctatgcctttaagctcaatacgattcaccaatgtatcgccctcgaacttcacttcagccctagtcttataattaccatcatccttaaaaaaaatggttctttcttggacgtaaccttctggcatcgccgatttgaaaaagtcatgttgtttcatgtgatccgggtagcgagcaaagcactggagaccgtatccaaacgtcgttacgagggtaggccagggcactggcaatttgcccgtggtacagataaacttgagggtcagctttccataagtagcatcaccttctccctcgcctgacacgctgaacttgtgaccattaacgtcgccgtccagctccaccaagataggcactacaccagtgaaaagctcctcgcccttcgacactggacccgggttttcttcgacatcgcctgcttgtttgagaagtgaaaagtttgtagcacccgatccgccttcccaaacgtaacccgatggcaagagttcccttattcccactgcagtgggctggccgtcgctatccttgacgatggctttaataccagggtgaagatccaagagcacttggcggcacctaccgcacggagaaagtattcctctattctcgttaccaatagccactatgcaagtcaagttgcctgccgcagccgccgctgctgttcccaggacgacgagttcggcgcatggtcctcccgtaaagtggtacacgttgactccggtgaagatacgtccatccgaagacaatgcggcgcttgcaacactataatcctctgaaataggaattgagtttatggttgcagtagcacgctcaatcagtgtgctctcttcttgagacagcggtttcgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacttgctagcttcttaagttagatctttatgcattacggacctttaattcaacccaacacaatatattatagttaaataagaattattatcaaatcatttgtatattaattaaaatactatactgtaaattacattttatttacaatcactcgacgaagacttgatcacccctagattcgaaagcggcccgctcactagtaacggccgccagtgtgctggaattcgcccttcgcggatcctgttaagatggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 7 - DNA, 7569 bp, synthetic construct
  misc_feature 1..7569: note = pCLD 047
  source 1..7569: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgtctcaggcttaattaatgatgcgatcgccgttacacgtttatgcgggccgaggcacatgcggcaggatgacggtccataccagattcctgggcacaactcatcggcaacgttccatcgtcctgctcctccacctcgggcgggaagccatggctcagttttttatagagctcatccattccgagtgttatacctgccgctgtcacaaattcaagcagcaccatgtggtcgcgtttctcgttcggatcctttgacagttttgattggtatgacaaatagtgattatctgggagcaggactggtccatctcctataggggtgttttgttgataatgatcggcaagttgcacgctaccgtcctctatgttgtggcgaatcttgaaatttacctttataccgtttttctgcttgtctgccataatatagacgttgtgcgagttataattgtattccaacttgtgtcccaatatgtttccatcttctttgaagtctatgcctttaagctcaatacgattcaccaatgtatcgccctcgaacttcacttcagccctagtcttataattaccatcatccttaaaaaaaatggttctttcttggacgtaaccttctggcatcgccgatttgaaaaagtcatgttgtttcatgtgatccgggtagcgagcaaagcactggagaccgtatccaaacgtcgttacgagggtaggccagggcactggcaatttgcccgtggtacagataaacttgagggtcagctttccataagtagcatcaccttctccctcgcctgacacgctgaacttgtgaccattaacgtcgccgtccagctccaccaagataggcactacaccagtgaaaagctcctcgcccttcgacactggacccgggttttcttcgacatcgcctgcttgtttgagaagtgaaaagtttgtagcacccgatccgccttcccaaacgtaacccgatggcaagagttcccttattcccactgcagtgggctggccgtcgctatccttgacgatggctttaataccagggtgaagatccaagagcacttggcggcacctaccgcacggagaaagtattcctctattctcgttaccaatagccactatgcaagtcaagttgcctgccgcagccgccgctgctgttcccaggacgacgagttcggcgcatggtcctcccgtaaagtggtacacgttgactccggtgaagatacgtccatccgaagacaatgcggcgcttgcaacactataatcctctgaaataggaattgagtttatggttgcagtagcacgctcaatcagtgtgctctcttcttgagacagcggtttcgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttgctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaacttgctagcttcttaagttagatctttatgcatatcatggagataattaaaatgataaccatctcgcaaataaataagtattttactgttttcgtaacagttttgtaataaaaaaacctataaatattccggattattcataccgtcccaccatcgggcgcggatctcggtcgccatggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 8 - DNA, 6326 bp, synthetic construct
  misc_feature 1..6326: note = pCLD 015
  source 1..6326: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttttgctagcttcttaagttagatctttatgcatttcggagcgagacctacggacctttaattcaacccaacacaatatattatagttaaataagaattattatcaaatcatttgtatattaattaaaatactatactgtaaattacattttatttacaatcactcgacgaagacttgatcacccctagattcgaaagcggcccgctcactagtaacggccgccagtgtgctggaattcgcccttcgcggatcctgttaagacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 9 - DNA, 7986 bp, synthetic construct
  misc_feature 1..7986: note = pCLD 020
  source 1..7986: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttggccgcgaattcactagtgattgcggaataattgccatatgtaaatgatgtcatcgttctaactcgctttacgagtagaattctacgtgtaaaacataatcaagagatgatgtcatttgtttttcaaaactgaactcaagaaatgatgtcatttgtttttcaaaactgaactggctttacgagcagaattctacttgtaacgcatgatcaagggatgatgtcatttgttttttaaaattgaactggctttacgagtagaattctacttgtaaaacacaatcgagagatgatgtcatattttgcacacggctctaattaaactcgctttacgagtaaaattctacttgtaacgcatgatcaagggatgatgtcattggatgagtcatttgtttttcaaaactaaactcgctttacgagtagaattctacttgtaaaacacaatcaagggatgatgtcattatacaaatgatgtcatttgtttttcaaaactaaactcgctttacgggtagaattctacttgtaaaacacaatcgagggatgatgtcatcctttacacatgattataaacgtgtttatgtatgactcatttgtttttcaaaactaaactcgctttacgagtagaattctacttgtaacgcacgatcaagggatgatgtcatttatttgtgcaaagctgatgtcatcttttgcacacgattataaacacaatcaaataatgactcatttgttttcaaaactgaactcgctttacgagtagaattctacttgtaaaacacaatcaagggatgatgtcattttaaaaatgatgtcatttgtttttcaaaactaaactcgctttacgagtagaattctacgtgtaaaacacaatcaagggatgatgtcatttactaaaataaaataattatttaaataaaaatgtttttattgtaaaatacacattgattacacgtgacaatcgaattcccgcttgctagcttcttaagttagatctttatgcatttcggagcgagaccaaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaacacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 10 - DNA, 10046 bp, synthetic construct
  misc_feature 1..10046: note = pCLD 050
  source 1..10046: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgttactgctcgaatatacaatcgtccaggtcaacgttcacaaggtcacacgcggtacaggcatccggtactttgcccataatgtggtggatataacagagcttttggtacgctttctttaccacacttacaggttgcgattcacttacagggaagcactccaggcagtccttttgaccatgggtaaaacatatgtttgagttttggttcatacgttcacactgacgacaagggaagagcatgaggttcataccgacatggcggctgcacttattctgataacgatcagcataattaatggaggcctcagcatcgctggtgctaggttgtgcgacgctttcacggacacgcttaggttcggagatgtcagcatcgctaggagcaggacgctttttagcacctcctttcttaacataaaactcgtgctcgacttctacgacatggtctttagcccagcgaaagaaatccttcacctcctgtttagtaactttaccgaaatcgtggtcgaggcgtctagtcagctcaaacttaaacattctatcctggaggggttgttgatgttcaaacgtcgtcgagttaccgtcaataactgcgcacatattagtattactcgtaacaattaccggggtaggatcgatttgagcggagcttttacacttttgatcgacacggactttggaaccacccaggattgccttcgcggattcaaccactttagctgtcattttaccttcttcccaccatatgaccattttatccacgcaatcattaaaagggaaattttcgttagtccaattcacacaaccataaaatgggacggtatgagcaatagcttcggcaatattagttttgccggttgtggctggaccgaagagccaaatagtattgcgtttaccaaatttctttgtagcccatccgaggaatacagacgcagcgtactgagggtcataaccattgagctccaagatcttgtaaattctgtttgaacttatgtcctcaactggttgttgaccgacgagatagtccggcgccgtctttgtaagcgacatgatcttaccagcgttgtccaacgctgcttttatctggcttctgctattactcgcagcgttaaagctaatataggatgcttgatcttcttgaatccattgtttttcgctcgttatgcctttatcgacaagccaacccaccaattccatgtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaactgcccatgaccactaaaatagagctgagcctagtcgcgtgatttgacttaagatttcggtctgtacgactatagtttcgtgagataaactggatcggcaatttgctagacgatctggcacagaaagacacagacgaaggactcactatggaatggcggcttcggccgccaatgatggatcccagcggaagtaggaagcgatgtggcctgaggaatacgtctgtcattgcgtttagataaggcatcccgctggaaagggaaccagtactttgtaaaggagctagaggttctctttatgctctgtggttcgtgcggttcttaaaagcaaacgacgagcgaacaggtgcgtgggcgagagggcaccctcatttctaagtacgatcaggtggactacgattgcattttatgggttccctgcggctgtcacgctaaatcactcggagaatggcaatgcaatcgactactagcaatgacccactgaccgaaatggagcataacgggtcgttttacacgtagaattctactcgtaaagcgagtttgtacgatgacatcatttttaaaatgacatcatccaccgatcttgcgttacaagtagaattctactcgtaaagcgagttcggttttgaaaaacaaatgacatcattctacaaatgacatcatccctcgattgtgttttacaagtagaattctactcgtaaagcgagttgaattttaaaaaaacaaatgacatcatcccttgatcatgcgttacaagtagaattctactcgtaaagcgagtttgtacgatgacatcatttttgcacaaacatatgagtcatatattaatcacgtttatcaacttgcacaaacaaatgagtcgtacattaatcatgtttatcaacttgcaaaaattaagttcaatgtaccatgtttaaacatgtttataatcacgtacaaaaatgacatcatttcttgattgtgttttacaagtaaaattctactcgtaaagcttttggtccgcccgagcggtgcaaccgattaggaccatgtacagcatttgttacaagtcttctagtaaacacaatcttcctgctcagtggcgcatgattatcgttgttgcaagccagcgtggtaagtaacagcaccactgcgagcctaatgtgccctttccacgaacacagggctgtccgatcctatattaggactccgcaatggggttagcaagtcgcaccctaaacgatgttgaagactcgcgatgtacatgctctggtacaatacatacgtgttccggctgttatcctgcatcggaacctcaatcatgcatcgcaccagcgtattcgtgtcatctaggagtgtcgcgtaggataaataattcaattaagatgtcgttatgctagtatacgcctacccgtcaccggccatctgtgtgcagatgagtcgacgagttactggccctgatttctccgcttctaataccacacactgggcaatacgagctcaagccagtctcttttgctagcttcttaagttagatctttatgcatttcggagcgagaccaaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaacacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 11 - DNA, 9979 bp, synthetic construct
  misc_feature 1..9979: note = pCLD 051
  source 1..9979: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgttactgctcgaatatacaatcgtccaggtcaacgttcacaaggtcacacgcggtacaggcatccggtactttgcccataatgtggtggatataacagagcttttggtacgctttctttaccacacttacaggttgcgattcacttacagggaagcactccaggcagtccttttgaccatgggtaaaacatatgtttgagttttggttcatacgttcacactgacgacaagggaagagcatgaggttcataccgacatggcggctgcacttattctgataacgatcagcataattaatggaggcctcagcatcgctggtgctaggttgtgcgacgctttcacggacacgcttaggttcggagatgtcagcatcgctaggagcaggacgctttttagcacctcctttcttaacataaaactcgtgctcgacttctacgacatggtctttagcccagcgaaagaaatccttcacctcctgtttagtaactttaccgaaatcgtggtcgaggcgtctagtcagctcaaacttaaacattctatcctggaggggttgttgatgttcaaacgtcgtcgagttaccgtcaataactgcgcacatattagtattactcgtaacaattaccggggtaggatcgatttgagcggagcttttacacttttgatcgacacggactttggaaccacccaggattgccttcgcggattcaaccactttagctgtcattttaccttcttcccaccatatgaccattttatccacgcaatcattaaaagggaaattttcgttagtccaattcacacaaccataaaatgggacggtatgagcaatagcttcggcaatattagttttgccggttgtggctggaccgaagagccaaatagtattgcgtttaccaaatttctttgtagcccatccgaggaatacagacgcagcgtactgagggtcataaccattgagctccaagatcttgtaaattctgtttgaacttatgtcctcaactggttgttgaccgacgagatagtccggcgccgtctttgtaagcgacatgatcttaccagcgttgtccaacgctgcttttatctggcttctgctattactcgcagcgttaaagctaatataggatgcttgatcttcttgaatccattgtttttcgctcgttatgcctttatcgacaagccaacccaccaattccattatattgtagcaaaaatttaatttttgccaaaagacttaataaatcatcagcaatggacaacaaagtaaataaaaaatttacaaagcataatagagaggtttattgattattgaacagcccatgaccactaaaatagagctgagcctagtcgcgtgatttgacttaagatttcggtctgtacgactatagtttcgtgagataaactggatcggcaatttgctagacgatctggcacagaaagacacagacgaaggactcactatggaatggcggcttcggccgccaatgatggatcccagcggaagtaggaagcgatgtggcctgaggaatacgtctgtcattgcgtttagataaggcatcccgctggaaagggaaccagtactttgtaaaggagctagaggttctctttatgctctgtggttcgtgcggttcttaaaagcaaacgacgagcgaacaggtgcgtgggcgagagggcaccctcatttctaagtacgatcaggtggactacgattgcattttatgggttccctgcggctgtcacgctaaatcactcggagaatggcaatgcaatcgactactagcaatgacccactgaccgaaatggagcataacgggtcgttttacacgtagaattctactcgtaaagcgagtttgtacgatgacatcatttttaaaatgacatcatccaccgatcttgcgttacaagtagaattctactcgtaaagcgagttcggttttgaaaaacaaatgacatcattctacaaatgacatcatccctcgattgtgttttacaagtagaattctactcgtaaagcgagttgaattttaaaaaaacaaatgacatcatcccttgatcatgcgttacaagtagaattctactcgtaaagcgagtttgtacgatgacatcatttttgcacaaacatatgagtcatatattaatcacgtttatcaacttgcacaaacaaatgagtcgtacattaatcatgtttatcaacttgcaaaaattaagttcaatgtaccatgtttaaacatgtttataatcacgtacaaaaatgacatcatttcttgattgtgttttacaagtaaaattctactcgtaaagcttttggtccgcccgagcggtgcaaccgattaggaccatgtacagcatttgttacaagtcttctagtaaacacaatcttcctgctcagtggcgcatgattatcgttgttgcaagccagcgtggtaagtaacagcaccactgcgagcctaatgtgccctttccacgaacacagggctgtccgatcctatattaggactccgcaatggggttagcaagtcgcaccctaaacgatgttgaagactcgcgatgtacatgctctggtacaatacatacgtgttccggctgttatcctgcatcggaacctcaatcatgcatcgcaccagcgtattcgtgtcatctaggagtgtcgcgtaggataaataattcaattaagatgtcgttatgctagtatacgcctacccgtcaccggccatctgtgtgcagatgagtcgacgagttactggccctgatttctccgcttctaataccacacactgggcaatacgagctcaagccagtctcttttgctagcttcttaagttagatctttatgcatttcggagcgagaccaaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaacacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 12 - DNA, 10101 bp, synthetic construct
  misc_feature 1..10101: note = pCLD 052
  source 1..10101: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgttactgctcgaatatacaatcgtccaggtcaacgttcacaaggtcacacgcggtacaggcatccggtactttgcccataatgtggtggatataacagagcttttggtacgctttctttaccacacttacaggttgcgattcacttacagggaagcactccaggcagtccttttgaccatgggtaaaacatatgtttgagttttggttcatacgttcacactgacgacaagggaagagcatgaggttcataccgacatggcggctgcacttattctgataacgatcagcataattaatggaggcctcagcatcgctggtgctaggttgtgcgacgctttcacggacacgcttaggttcggagatgtcagcatcgctaggagcaggacgctttttagcacctcctttcttaacataaaactcgtgctcgacttctacgacatggtctttagcccagcgaaagaaatccttcacctcctgtttagtaactttaccgaaatcgtggtcgaggcgtctagtcagctcaaacttaaacattctatcctggaggggttgttgatgttcaaacgtcgtcgagttaccgtcaataactgcgcacatattagtattactcgtaacaattaccggggtaggatcgatttgagcggagcttttacacttttgatcgacacggactttggaaccacccaggattgccttcgcggattcaaccactttagctgtcattttaccttcttcccaccatatgaccattttatccacgcaatcattaaaagggaaattttcgttagtccaattcacacaaccataaaatgggacggtatgagcaatagcttcggcaatattagttttgccggttgtggctggaccgaagagccaaatagtattgcgtttaccaaatttctttgtagcccatccgaggaatacagacgcagcgtactgagggtcataaccattgagctccaagatcttgtaaattctgtttgaacttatgtcctcaactggttgttgaccgacgagatagtccggcgccgtctttgtaagcgacatgatcttaccagcgttgtccaacgctgcttttatctggcttctgctattactcgcagcgttaaagctaatataggatgcttgatcttcttgaatccattgtttttcgctcgttatgcctttatcgacaagccaacccaccaattccatcttaacaggatccgcgaagggcgaattccagcacactggcggccgttactagtgagcgggccgctttcgaatctaggggtgatcaagtcttcgtcgagtgattgtaaataaaatgtaatttacagtatagtattttaattaatatacaaatgatttgataataattcttatttaactataatatattgtgttgggttgaattaaaggtccgtagcccatgaccactaaaatagagctgagcctagtcgcgtgatttgacttaagatttcggtctgtacgactatagtttcgtgagataaactggatcggcaatttgctagacgatctggcacagaaagacacagacgaaggactcactatggaatggcggcttcggccgccaatgatggatcccagcggaagtaggaagcgatgtggcctgaggaatacgtctgtcattgcgtttagataaggcatcccgctggaaagggaaccagtactttgtaaaggagctagaggttctctttatgctctgtggttcgtgcggttcttaaaagcaaacgacgagcgaacaggtgcgtgggcgagagggcaccctcatttctaagtacgatcaggtggactacgattgcattttatgggttccctgcggctgtcacgctaaatcactcggagaatggcaatgcaatcgactactagcaatgacccactgaccgaaatggagcataacgggtcgttttacacgtagaattctactcgtaaagcgagtttgtacgatgacatcatttttaaaatgacatcatccaccgatcttgcgttacaagtagaattctactcgtaaagcgagttcggttttgaaaaacaaatgacatcattctacaaatgacatcatccctcgattgtgttttacaagtagaattctactcgtaaagcgagttgaattttaaaaaaacaaatgacatcatcccttgatcatgcgttacaagtagaattctactcgtaaagcgagtttgtacgatgacatcatttttgcacaaacatatgagtcatatattaatcacgtttatcaacttgcacaaacaaatgagtcgtacattaatcatgtttatcaacttgcaaaaattaagttcaatgtaccatgtttaaacatgtttataatcacgtacaaaaatgacatcatttcttgattgtgttttacaagtaaaattctactcgtaaagcggaccaggaccaggaccgctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacttttgctagcttcttaagttagatctttatgcatttcggagcgagaccaaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaacacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 13 - DNA, 10072 bp, synthetic construct
  misc_feature 1..10072: note = pCLD 053
  source 1..10072: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgttactgctcgaatatacaatcgtccaggtcaacgttcacaaggtcacacgcggtacaggcatccggtactttgcccataatgtggtggatataacagagcttttggtacgctttctttaccacacttacaggttgcgattcacttacagggaagcactccaggcagtccttttgaccatgggtaaaacatatgtttgagttttggttcatacgttcacactgacgacaagggaagagcatgaggttcataccgacatggcggctgcacttattctgataacgatcagcataattaatggaggcctcagcatcgctggtgctaggttgtgcgacgctttcacggacacgcttaggttcggagatgtcagcatcgctaggagcaggacgctttttagcacctcctttcttaacataaaactcgtgctcgacttctacgacatggtctttagcccagcgaaagaaatccttcacctcctgtttagtaactttaccgaaatcgtggtcgaggcgtctagtcagctcaaacttaaacattctatcctggaggggttgttgatgttcaaacgtcgtcgagttaccgtcaataactgcgcacatattagtattactcgtaacaattaccggggtaggatcgatttgagcggagcttttacacttttgatcgacacggactttggaaccacccaggattgccttcgcggattcaaccactttagctgtcattttaccttcttcccaccatatgaccattttatccacgcaatcattaaaagggaaattttcgttagtccaattcacacaaccataaaatgggacggtatgagcaatagcttcggcaatattagttttgccggttgtggctggaccgaagagccaaatagtattgcgtttaccaaatttctttgtagcccatccgaggaatacagacgcagcgtactgagggtcataaccattgagctccaagatcttgtaaattctgtttgaacttatgtcctcaactggttgttgaccgacgagatagtccggcgccgtctttgtaagcgacatgatcttaccagcgttgtccaacgctgcttttatctggcttctgctattactcgcagcgttaaagctaatataggatgcttgatcttcttgaatccattgtttttcgctcgttatgcctttatcgacaagccaacccaccaattccatgtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaactgcccatgaccactaaaatagagctgagcctagtcgcgtgatttgacttaagatttcggtctgtacgactatagtttcgtgagataaactggatcggcaatttgctagacgatctggcacagaaagacacagacgaaggactcactatggaatggcggcttcggccgccaatgatggatcccagcggaagtaggaagcgatgtggcctgaggaatacgtctgtcattgcgtttagataaggcatcccgctggaaagggaaccagtactttgtaaaggagctagaggttctctttatgctctgtggttcgtgcggttcttaaaagcaaacgacgagcgaacaggtgcgtgggcgagagggcaccctcatttctaagtacgatcaggtggactacgattgcattttatgggttccctgcggctgtcacgctaaatcactcggagaatggcaatgcaatcgactactagcaatgacccactgaccgaaatggagcataacgggtcgttttacacgtagaattctactcgtaaagcgagtttgtacgatgacatcatttttaaaatgacatcatccaccgatcttgcgttacaagtagaattctactcgtaaagcgagttcggttttgaaaaacaaatgacatcattctacaaatgacatcatccctcgattgtgttttacaagtagaattctactcgtaaagcgagttgaattttaaaaaaacaaatgacatcatcccttgatcatgcgttacaagtagaattctactcgtaaagcgagtttgtacgatgacatcatttttgcacaaacatatgagtcatatattaatcacgtttatcaacttgcacaaacaaatgagtcgtacattaatcatgtttatcaacttgcaaaaattaagttcaatgtaccatgtttaaacatgtttataatcacgtacaaaaatgacatcatttcttgattgtgttttacaagtaaaattctactcgtaaagcggaccaggaccaggaccgctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacttttgctagcttcttaagttagatctttatgcatttcggagcgagaccaaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaacacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 14 - DNA, 10005 bp, synthetic construct
  misc_feature 1..10005: note = pCLD 054
  source 1..10005: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatgtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgttactgctcgaatatacaatcgtccaggtcaacgttcacaaggtcacacgcggtacaggcatccggtactttgcccataatgtggtggatataacagagcttttggtacgctttctttaccacacttacaggttgcgattcacttacagggaagcactccaggcagtccttttgaccatgggtaaaacatatgtttgagttttggttcatacgttcacactgacgacaagggaagagcatgaggttcataccgacatggcggctgcacttattctgataacgatcagcataattaatggaggcctcagcatcgctggtgctaggttgtgcgacgctttcacggacacgcttaggttcggagatgtcagcatcgctaggagcaggacgctttttagcacctcctttcttaacataaaactcgtgctcgacttctacgacatggtctttagcccagcgaaagaaatccttcacctcctgtttagtaactttaccgaaatcgtggtcgaggcgtctagtcagctcaaacttaaacattctatcctggaggggttgttgatgttcaaacgtcgtcgagttaccgtcaataactgcgcacatattagtattactcgtaacaattaccggggtaggatcgatttgagcggagcttttacacttttgatcgacacggactttggaaccacccaggattgccttcgcggattcaaccactttagctgtcattttaccttcttcccaccatatgaccattttatccacgcaatcattaaaagggaaattttcgttagtccaattcacacaaccataaaatgggacggtatgagcaatagcttcggcaatattagttttgccggttgtggctggaccgaagagccaaatagtattgcgtttaccaaatttctttgtagcccatccgaggaatacagacgcagcgtactgagggtcataaccattgagctccaagatcttgtaaattctgtttgaacttatgtcctcaactggttgttgaccgacgagatagtccggcgccgtctttgtaagcgacatgatcttaccagcgttgtccaacgctgcttttatctggcttctgctattactcgcagcgttaaagctaatataggatgcttgatcttcttgaatccattgtttttcgctcgttatgcctttatcgacaagccaacccaccaattccattatattgtagcaaaaatttaatttttgccaaaagacttaataaatcatcagcaatggacaacaaagtaaataaaaaatttacaaagcataatagagaggtttattgattattgaacagcccatgaccactaaaatagagctgagcctagtcgcgtgatttgacttaagatttcggtctgtacgactatagtttcgtgagataaactggatcggcaatttgctagacgatctggcacagaaagacacagacgaaggactcactatggaatggcggcttcggccgccaatgatggatcccagcggaagtaggaagcgatgtggcctgaggaatacgtctgtcattgcgtttagataaggcatcccgctggaaagggaaccagtactttgtaaaggagctagaggttctctttatgctctgtggttcgtgcggttcttaaaagcaaacgacgagcgaacaggtgcgtgggcgagagggcaccctcatttctaagtacgatcaggtggactacgattgcattttatgggttccctgcggctgtcacgctaaatcactcggagaatggcaatgcaatcgactactagcaatgacccactgaccgaaatggagcataacgggtcgttttacacgtagaattctactcgtaaagcgagtttgtacgatgacatcatttttaaaatgacatcatccaccgatcttgcgttacaagtagaattctactcgtaaagcgagttcggttttgaaaaacaaatgacatcattctacaaatgacatcatccctcgattgtgttttacaagtagaattctactcgtaaagcgagttgaattttaaaaaaacaaatgacatcatcccttgatcatgcgttacaagtagaattctactcgtaaagcgagtttgtacgatgacatcatttttgcacaaacatatgagtcatatattaatcacgtttatcaacttgcacaaacaaatgagtcgtacattaatcatgtttatcaacttgcaaaaattaagttcaatgtaccatgtttaaacatgtttataatcacgtacaaaaatgacatcatttcttgattgtgttttacaagtaaaattctactcgtaaagcggaccaggaccaggaccgctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacttttgctagcttcttaagttagatctttatgcatttcggagcgagaccaaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaacacggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 15 - DNA, 1194 bp, synthetic construct
  misc_feature 1..1194: note = Extreme codon optimized Rep52
  source 1..1194: mol_type = other DNA
atggaattggtgggttggcttgtcgataaaggcataacgagcgaaaaacaatggattcaagaagatcaagcatcctatattagctttaacgctgcgagtaatagcagaagccagataaaagcagcgttggacaacgctggtaagatcatgtcgcttacaaagacggcgccggactatctcgtcggtcaacaaccagttgaggacataagttcaaacagaatttacaagatcttggagctcaatggttatgaccctcagtacgctgcgtctgtattcctcggatgggctacaaagaaatttggtaaacgcaatactatttggctcttcggtccagccacaaccggcaaaactaatattgccgaagctattgctcataccgtcccattttatggttgtgtgaattggactaacgaaaatttcccttttaatgattgcgtggataaaatggtcatatggtgggaagaaggtaaaatgacagctaaagtggttgaatccgcgaaggcaatcctgggtggttccaaagtccgtgtcgatcaaaagtgtaaaagctccgctcaaatcgatcctaccccggtaattgttacgagtaatactaatatgtgcgcagttattgacggtaactcgacgacgtttgaacatcaacaacccctccaggatagaatgtttaagtttgagctgactagacgcctcgaccacgatttcggtaaagttactaaacaggaggtgaaggatttctttcgctgggctaaagaccatgtcgtagaagtcgagcacgagttttatgttaagaaaggaggtgctaaaaagcgtcctgctcctagcgatgctgacatctccgaacctaagcgtgtccgtgaaagcgtcgcacaacctagcaccagcgatgctgaggcctccattaattatgctgatcgttatcagaataagtgcagccgccatgtcggtatgaacctcatgctcttcccttgtcgtcagtgtgaacgtatgaaccaaaactcaaacatatgttttacccatggtcaaaaggactgcctggagtgcttccctgtaagtgaatcgcaacctgtaagtgtggtaaagaaagcgtaccaaaagctctgttatatccaccacattatgggcaaagtaccggatgcctgtaccgcgtgtgaccttgtgaacgttgacctggacgattgtatattcgagcagtaa

SEQ ID NO: 16 - DNA, 8316 bp, synthetic construct
  misc_feature 1..8316: note = pCLD 011
  source 1..8316: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcataccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagtcgagggcccgcggtaccgtcgatgaatccatggttcgcaattctgcacaggtgaagaccaagcaacacggacttaaccctcccaaacataaccagaggggagaagttcacgaataccgacggcggttggctggccgtctgaatcttttacaatagctttaatgcctggatgaaggtcaagaagtacctgacgacacctgccgcatggggaaagaataccacggttttcatttccaatcgccacgatacacgtaaggttacccgctgctgccgcagccgcagtacccaatacgaccagctccgcacaagggcctccggtgaaatggtacacgttaacacccgtgaagatgcgtccatccgacgagagcgccgcacttgcaactgagtaatcttccgaaattggtatagaattgatcgtggcggtagcacgctcaatcaatgtagattcctcctgtgagaggggttttgccatggtggcgaccggtagcctcgagtaccggatcctctagcggccgaacagatgctgttcaactgtgtttaccagatcgttgcgggctgtatttataggcgcgataagcgggacgggcgcctcgtgtccggtcacgcgcatgagataacgcgcggctgatatggaggcgcgtcctgttccgataaggagttgcgtccggctgcggttagcaacacaggaagctggcgtcctgtcacgataagacaacactcgtccggtccgataatgtgattcgtacgtgacaggacgcgacccgataaggccggcctacgtgactgccgacacgtacttttttgcactgcaaaaaggttcaatgtgtggtagtgtatttggagcgtatacaacggtgtagactatttatgtaaaatagtctacgaaacgtagagtttgtactatgtatgggcccgcgtgcaaaagcgtgtttttttgcagtgcaaaaaagttggtggtggggaggccaccgagtataaaggtgcttgttggcaaacatgaaaacacagttcaacagaattgttgttgaagcaacattagcaccatacattgtttatcatcatgaataacttcgtataatgtatgctatacgaagttatttgcggccgcttgatatcttcctgcaggttatcgatttggccgcgaattcactagtgattgcggaataattgccatatgtaaatgatgtcatcgttctaactcgctttacgagtagaattctacgtgtaaaacataatcaagagatgatgtcatttgtttttcaaaactgaactcaagaaatgatgtcatttgtttttcaaaactgaactggctttacgagcagaattctacttgtaacgcatgatcaagggatgatgtcatttgttttttaaaattgaactggctttacgagtagaattctacttgtaaaacacaatcgagagatgatgtcatattttgcacacggctctaattaaactcgctttacgagtaaaattctacttgtaacgcatgatcaagggatgatgtcattggatgagtcatttgtttttcaaaactaaactcgctttacgagtagaattctacttgtaaaacacaatcaagggatgatgtcattatacaaatgatgtcatttgtttttcaaaactaaactcgctttacgggtagaattctacttgtaaaacacaatcgagggatgatgtcatcctttacacatgattataaacgtgtttatgtatgactcatttgtttttcaaaactaaactcgctttacgagtagaattctacttgtaacgcacgatcaagggatgatgtcatttatttgtgcaaagctgatgtcatcttttgcacacgattataaacacaatcaaataatgactcatttgttttcaaaactgaactcgctttacgagtagaattctacttgtaaaacacaatcaagggatgatgtcattttaaaaatgatgtcatttgtttttcaaaactaaactcgctttacgagtagaattctacgtgtaaaacacaatcaagggatgatgtcatttactaaaataaaataattatttaaataaaaatgtttttattgtaaaatacacattgattacacgtgacaatcgaattcccgcttgctagcttcttaagttagatctgacccgttatgctccatttcggtcagtgggtcattgctagtagtcgattgcattgccattctccgagtgatttagcgtgacagccgcagggaacccataaaatgcaatcgtagtccacctgatcgtacttagaaatgagggtgccctctcgcccacgcacctgttcgctcgtcgtttgcttttaagaaccgcacgaaccacagagcataaagagaacctctagctcctttacaaagtactggttccctttccagcgggatgccttatctaaacgcaatgacagacgtattcctcaggccacatcgcttcctacttccgctgggatccatcattggcggccgaagccgccattccatagtgagtccttcgtctgtgtctttctgtgccagatcgtctagcaaattgccgatccagtttatctcacgaaactatagtcgtacagaccgaaatcttaagtcaaatcacgcgactaggctcagctctattttagtggtcatgggttttggtccgcccgagcggtgcaaccgattaggaccatgtacagcatttgttacaagtcttctagtaaacacaatcttcctgctcagtggcgcatgattatcgttgttgctagccagcgtggtaagtaacagcaccactgcgagcctaatgtgccctttccacgaacacagggctgtccgatcctatattaggactccgcaatggggttagcaagtcgcaccctaaacgatgttgaagactcgcgatgtacatgctctggtacaatacatacgtgttccggctgttatcctgcatcggaacctcaatcatgcatcgcaccagcgtattcgtgtcatctaggagtgtcgcgtaggataaataattcaattaagatgtcgttatgctagtatacgcctacccgtcaccggccatctgtgtgcagatgagtcgacgagttactggccctgatttctccgcttctaataccacacactgggcaatacgagctcaagccagtctcgatctttatgcatttcggagcgagaccatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtaacggccgccagtgtgctggaattcgcccttcgcggatcctgttaagatggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgatgaagagtaactaagggcgaattccagcacactggcggccgttactaggtagctgagcgggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcggtctcaccatgcgtacagcttgacgcgtgcgtaataacttcgtataatgtatgctatacgaagttatactgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 17 - DNA, 1866 bp, synthetic construct
  misc_feature 1..1866: note = ATG - AAV2 Rep78/52
  source 1..1866: mol_type = other DNA
atggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 18 - DNA, 1866 bp, synthetic construct
  misc_feature 1..1866: note = ACG - AAV2 Rep78/52
  source 1..1866: mol_type = other DNA
acggcggggttctacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 19 - DNA, 486 bp, synthetic construct
  misc_feature 1..486: note = Baculovirus hr4b
  source 1..486: mol_type = other DNA
gctttacgagtagaattttacttgtaaaacacaatcaagaaatgatgtcatttttgtacgtgattataaacatgtttaaacatggtacattgaacttaatttttgcaagttgataaacatgattaatgtacgactcatttgtttgtgcaagttgataaacgtgattaatatatgactcatatgtttgtgcaaaaatgatgtcatcgtacaaactcgctttacgagtagaattctacttgtaacgcatgatcaagggatgatgtcatttgtttttttaaaattcaactcgctttacgagtagaattctacttgtaaaacacaatcgagggatgatgtcatttgtagaatgatgtcatttgtttttcaaaaccgaactcgctttacgagtagaattctacttgtaacgcaagatcggtggatgatgtcattttaaaaatgatgtcatcgtacaaactcgctttacgagtagaattctacgtgtaaaac

SEQ ID NO: 20 - DNA, 509 bp, synthetic construct
  misc_feature 1..509: note = Baculovirus hr5
  source 1..509: mol_type = other DNA
gctttacgagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaac

SEQ ID NO: 21 - DNA, 906 bp, synthetic construct
  misc_feature 1..906: note = 39k promoter full-length
  source 1..906: mol_type = other DNA
aaggctgtcttgctgtgtgcccgtcgcgcgtaccggagcgcgaacgcgcccgccgccgacataaacgacacttttctagaaaaaatttccataccacgaggtcatcgcgattgttgcgacgcaaaagtttacgaaactgccgtgcgcgagtttgtggaagaaactggccggttttttgacagcgcgttcatctacaagtttccatttacgttacaatggaaagacgacggtgtcacttacaagtatttgatatacgtaggcgtcgtgcgcggcaacttgattgacgtgaacgccaaacccaacacgtacaccgtgaagttgttgccgggcacgtttggcaacgactatcgtataatgttaaaaccgcgacgcttcaattgcgaaataacgcgcagcctggccatcgtgccgctcaacaaatattttaattatatgaacgacaaacaattgatcacgtacgattacagcaattacattgaattttttagttttgtgcgcagcatcaagaagcgtttcgataataggcaattgcaagactttttctacgccactctaaaaaagatagacaacaacgatgccccccaaaaattgcacgcacttaggcgggtgtaattcggactgcttgacccgaagcgaaatacaagcgctgttcagggaagccatcaacacgctcaagcacacaatgaatacagaagacgtctgcgcgcacatgttggacatcgtgtcgtttgagcgtataaaagaatatataagagctaatttaggccatttcacagtaattaccgacaaatgttctaagcgtaaggtgtgtcttcatcacaaacgaattgccaggctgttgggcattaaaaaaatatatcatcaagaatacaagcgggtcgtttcaaaggtttacaaaaatcaaac

SEQ ID NO: 22 - DNA, 213 bp, synthetic construct
  misc_feature 1..213: note = p10 promoter
  source 1..213: mol_type = other DNA
tacggacctttaattcaacccaacacaatatattatagttaaataagaattattatcaaatcatttgtatattaattaaaatactatactgtaaattacattttatttacaatcactcgacgaagacttgatcacccctagattcgaaagcggcccgctcactagtaacggccgccagtgtgctggaattcgcccttcgcggatcctgttaag

SEQ ID NO: 23 - DNA, 184 bp, synthetic construct
  misc_feature 1..184: note = p6.9 promoter
  source 1..184: mol_type = other DNA
agttcaacattgaggattacaataacatatttaaggtgatggaaaatattaggaaacacagcaacaaaaattcaaacgaccaagacgagttaaacatatatttgggagttcagtcgtcgaatgcaaagcgtaaaaaatattaataaggtaaaaattacagctacataaattacacaatttaaac

SEQ ID NO: 24 - DNA, 117 bp, synthetic construct
  misc_feature 1..117: note = pSel120 promoter
  source 1..117: mol_type = other DNA
tgttcaataatcaataaacctctctattatgctttgtaaattttttatttactttgttgtccattgctgatgatttattaagtcttttggcaaaaattaaatttttgctacaatata

SEQ ID NO: 25 - DNA, 129 bp, synthetic construct
  misc_feature 1..129: note = polH promoter
  source 1..129: mol_type = other DNA
atcatggagataattaaaatgataaccatctcgcaaataaataagtattttactgttttcgtaacagttttgtaataaaaaaacctataaatattccggattattcataccgtcccaccatcgggcgcg

SEQ ID NO: 26 - DNA, 457 bp, synthetic construct
  misc_feature 1..457: note = Baculovirus hr1
  source 1..457: mol_type = other DNA
gttttacaagtagaattctacccgtaaagcgagtttagttttgaaaaacaaatgacatcatttgtataatgacatcatcccctgattgtgttttacaagtagaattctatccgtaaagcgagttcagttttgaaaacaaatgagtcatacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgagccgtgtgcaaaacatgacatcagcttatgagtcataattaatcgtgcgttacaagtagaattctactcgtaaagc

SEQ ID NO: 27 - DNA, 666 bp, synthetic construct
  misc_feature 1..666: note = Baculovirus hr3
  source 1..666: mol_type = other DNA
gatttacgcgtagaattctacttgtaaagcaagttaaaataagccgtgtgcaaaaatgacatcagacaaatgacatcatctacctatcatgatcatgttaataatcatgttttaaaatgacatcagcttatgactaataattgatcgtgcgttacaagtagaattctactcgtaaagcgagtttagttttgaaaaacaaatgagtcatcattaaacatgttaataatcgtgtataaaggatgacatcatccactaatcgtgcgttacaagtagaattctactcgtaaagcgagttcggttttgaaaaacaaatgacatcatttcttgattgtgttttacacgtagaattctactcgtaaagtatgttcagtttaaaaaacaaatgacatcattttacagatgacatcatttcttgattatgttttacaagtagaattctactcgtaaagcaagtttagttttaaaaaacaaatgacatcatctcttgattatgttttacaagtagaattctactcgtaaagcgagtttagttttgaaaaacaaatgacatcatctcttgattatgttttacaagtagaattctactcgtaaagcgagtttagttttcaaaaacaaatgacatcatcccttgatcatgcgttacaagtagaattctactcgtaaagc

SEQ ID NO: 28 - DNA, 806 bp, synthetic construct
  misc_feature 1..806: note = Synthetic hr134b5
  source 1..806: mol_type = other DNA
gatttacgcgtagaattctacttgtaaagcaagttaaaataagccgtgtgcaaaaatgacatcagacaaatgacatcatctacctatcatgatcatgttaataatcatgttttaaaatgacatcagcttatgactaataattgatcgtgcgttacaagtagaattctacgcgtaaaacacaatcaagtatgagtcataatctgatgtcatgttttgtacacggctcataaccgaactggctttacgagtagaattctacttgtaatgcacgatcagtggatgatgtcatttgtttttcaaatcgagatgatgtcatgtttatgacatcatccactaatcgtgcgttacaagtagaattctactcgtaaagcgagttcggttttgaaaaacaaatgacatcatacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgatgtcatttgttataaaaataaaagctgatgtcatgttttgcacatggctcataactaaactcgctttacgggtagaattctacttgtaacgcaagatcggtggatgatgtcattttaaaaatgatgtcatcgtacaaactcgctttacgagtagaattctacgtgtaaaac

SEQ ID NO: 29 - DNA, 14265 bp, synthetic construct
  misc_feature 1..14265: note = polH Cap2/5 nano-luciferase
  source 1..14265: mol_type = other DNA
tggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggctgcaggggggggggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcttcggtcgacggatccttatcgattttaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggccgccccgactctagaattacgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagactggggcagggaaggcagcaggcaacaccaggagcaggcccagggagaaggcaactggaccgaaggcgcttgtggagaaggagttcatggtggctttaccaacagtaccggattgccaagcttggccgccgaggcccactgactgcgttagcaatttaactgtgataaactaccgcaataaagcttctagtgatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgccaacatggcggtcatattggacatgagccaatataaatgtacatattatgatatagatacaacgtatgcaatggccaatagccaatattgatttatgctatataaccaatgaataatatggctaatggccaatattgaggccagttagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgtgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagcttcctgttaagacggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaaaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccacgtagtgggccatcgccctgatagacggtttttcgccctttgacgttaattaattaagtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgcgtcgatttttgtgatgctcgtcaggggggcggagcctatggaaaaacgccagcaacgcggcctttttacggttcctggccttttgctggccttttgctcacatgttctttcctgcgttatcccctgattctg

SEQ ID NO: 30 - DNA, 2178 bp, synthetic construct
  misc_feature 1..2178: note = Cap AAV2/5
  source 1..2178: mol_type = other DNA
acggctgccgacggttatctacccgattggttggaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 31 - DNA, 2178 bp, synthetic construct
  misc_feature 1..2178: note = Cap AAV5
  source 1..2178: mol_type = other DNA
acggcttcttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 32 - DNA, 28 bp, synthetic construct
  misc_feature 1..28: note = hr 28-mer
  source 1..28: mol_type = other DNA
ctttacgagtagaattctacgcgtaaaa

SEQ ID NO: 33 - DNA, 1194 bp, synthetic construct
  misc_feature 1..1194: note = Rep52 wt
  source 1..1194: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 34 - DNA, 1194 bp, synthetic construct
  misc_feature 1..1194: note = rep52 sf9 (insect cell) optimised
  source 1..1194: mol_type = other DNA
atggagctggtgggttggctggtggacaagggtatcacctccgagaagcagtggatccaggaggaccaggcttcctacatctccttcaacgctgcttccaactcccgttcccagatcaaggctgctctggacaacgctggtaagatcatgtccctgaccaagaccgctcctgactacctggtgggtcagcagcctgtggaggacatctcctccaaccgtatctacaagatcctggagctgaacggttacgaccctcagtacgctgcttccgtgttcctgggttgggctaccaagaagttcggtaagcgtaacaccatctggctgttcggtcctgctaccaccggtaagaccaacatcgctgaggctatcgctcacaccgtgcctttctacggttgcgtgaactggaccaacgagaacttccctttcaacgactgcgtggacaagatggtgatctggtgggaggagggtaagatgaccgctaaggtggtggagtccgctaaggctatcctgggtggttccaaggtgcgtgtggaccagaagtgcaagtcctccgctcagatcgaccctacccctgtgatcgtgacctccaacaccaacatgtgcgctgtgatcgacggtaactccaccaccttcgagcaccagcagcctctgcaggaccgtatgttcaagttcgagctgacccgtcgtctggaccacgacttcggtaaggtgaccaagcaggaggtgaaggacttcttccgttgggctaaggaccacgtggtggaggtggagcacgagttctacgtgaagaagggtggtgctaagaagcgtcctgctccttccgacgctgacatctccgagcctaagcgtgtgcgtgagtccgtggctcagccttccacctccgacgctgaggcttccatcaactacgctgaccgttaccagaacaagtgctcccgtcacgtgggtatgaacctgatgctgttcccttgccgtcagtgcgagcgtatgaaccagaactccaacatctgcttcacccacggtcagaaggactgcctggagtgcttccctgtgtccgagtcccagcctgtgtccgtggtgaagaaggcttaccagaagctgtgctacatccaccacatcatgggtaaggtgcctgacgcttgcaccgcttgcgacctggtgaacgtggacctggacgactgcatcttcgagcagtaa

SEQ ID NO: 35 - DNA, 1194 bp, synthetic construct
  misc_feature 1..1194: note = rep52 AT optimised
  source 1..1194: mol_type = other DNA
atggaattagtaggatggttagtagataaaggaataacatcagaaaaacaatggatacaagaagatcaagcatcatatatatcatttaatgcagcatcaaattcaagatcacaaataaaagcagcattagataatgcaggaaaaataatgtcattaacaaaaacagcaccagattatttagtaggacaacaaccagtagaagatatatcatcaaatagaatatataaaatattagaattaaatggatatgatccacaatatgcagcatcagtatttttaggatgggcaacaaaaaaatttggaaaaagaaatacaatatggttatttggaccagcaacaacaggaaaaacaaatatagcagaagcaatagcacatacagtaccattttatggatgtgtaaattggacaaatgaaaattttccatttaatgattgtgtagataaaatggtaatatggtgggaagaaggaaaaatgacagcaaaagtagtagaatcagcaaaagcaatattaggaggatcaaaagtaagagtagatcaaaaatgtaaatcatcagcacaaatagatccaacaccagtaatagtaacatcaaatacaaatatgtgtgcagtaatagatggaaattcaacaacatttgaacatcaacaaccattacaagatagaatgtttaaatttgaattaacaagaagattagatcatgattttggaaaagtaacaaaacaagaagtaaaagatttttttagatgggcaaaagatcatgtagtagaagtagaacatgaattttatgtaaaaaaaggaggagcaaaaaaaagaccagcaccatcagatgcagatatatcagaaccaaaaagagtaagagaatcagtagcacaaccatcaacatcagatgcagaagcatcaataaattatgcagatagatatcaaaataaatgttcaagacatgtaggaatgaatttaatgttatttccatgtagacaatgtgaaagaatgaatcaaaattcaaatatatgttttacacatggacaaaaagattgtttagaatgttttccagtatcagaatcacaaccagtatcagtagtaaaaaaagcatatcaaaaattatgttatatacatcatataatgggaaaagtaccagatgcatgtacagcatgtgatttagtaaatgtagatttagatgattgtatatttgaacaataa

SEQ ID NO: 36 - DNA, 1194 bp, synthetic construct
  misc_feature 1..1194: note = rep52 GC optimised
  source 1..1194: mol_type = other DNA
atggagctggtggggtggctggtggacaaggggatcacgagcgagaagcagtggatccaggaggaccaggcgagctacatcagcttcaacgcggcgagcaacagccggagccagatcaaggcggcgctggacaacgcggggaagatcatgagcctgacgaagacggcgccggactacctggtggggcagcagccggtggaggacatcagcagcaaccggatctacaagatcctggagctgaacgggtacgacccgcagtacgcggcgagcgtgttcctggggtgggcgacgaagaagttcgggaagcggaacacgatctggctgttcgggccggcgacgacggggaagacgaacatcgcggaggcgatcgcgcacacggtgccgttctacgggtgcgtgaactggacgaacgagaacttcccgttcaacgactgcgtggacaagatggtgatctggtgggaggaggggaagatgacggcgaaggtggtggagagcgcgaaggcgatcctgggggggagcaaggtgcgggtggaccagaagtgcaagagcagcgcgcagatcgacccgacgccggtgatcgtgacgagcaacacgaacatgtgcgcggtgatcgacgggaacagcacgacgttcgagcaccagcagccgctgcaggaccggatgttcaagttcgagctgacgcggcggctggaccacgacttcgggaaggtgacgaagcaggaggtgaaggacttcttccggtgggcgaaggaccacgtggtggaggtggagcacgagttctacgtgaagaaggggggggcgaagaagcggccggcgccgagcgacgcggacatcagcgagccgaagcgggtgcgggagagcgtggcgcagccgagcacgagcgacgcggaggcgagcatcaactacgcggaccggtaccagaacaagtgcagccggcacgtggggatgaacctgatgctgttcccgtgccggcagtgcgagcggatgaaccagaacagcaacatctgcttcacgcacgggcagaaggactgcctggagtgcttcccggtgagcgagagccagccggtgagcgtggtgaagaaggcgtaccagaagctgtgctacatccaccacatcatggggaaggtgccggacgcgtgcacggcgtgcgacctggtgaacgtggacctggacgactgcatcttcgagcagtaa

SEQ ID NO: 37 - DNA, 1194 bp, synthetic construct; identical to SEQ ID NO: 33
  misc_feature 1..1194: note = Rep52
  source 1..1194: mol_type = other DNA

SEQ ID NO: 38 - protein, 397 aa, synthetic construct
  REGION 1..397: note = Rep52
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 39 - DNA, 1873 bp, synthetic construct
  misc_feature 1..1873: note = Rep78
  source 1..1873: mol_type = other DNA
cgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaa

SEQ ID NO: 40 - protein, 621 aa, synthetic construct
  REGION 1..621: note = Rep78
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 41 - protein, 738 aa, synthetic construct
  REGION 1..738: note = AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 42 - DNA, 250 bp, synthetic construct
  misc_feature 1..250: note = PolH long
  source 1..250: mol_type = other DNA
tgtaatgagacgcacaaactaatatcacaaactggaaatgtctatcaatatatagttgctgatctatgcatcagctgctagtactccggaatattaatagatcatggagataattaaaatgataaccatctcgcaaataaataagtattttactgttttcgtaacagttttgtaataaaaaaacctataaatattccggattattcataccgtcccaccatcgggcgcggatcgtaccgggcccaagctt

SEQ ID NO: 43 - DNA, 155 bp, synthetic construct
  misc_feature 1..155: note = PolH short
  source 1..155: mol_type = other DNA
tgtaatgagacgcacaaactaatatcacaaactggaaatgtctatcaatatatagttgctgatatcatggagataattaaaatgataaccatctcgcaaataaataagtattttactgttttcgtaacagttttgtaataaaaaaacctataaat

SEQ ID NO: 44 - DNA, 2208 bp, synthetic construct
  misc_feature 1..2208: note = AAV 1; VP1, VP2, VP3; startcodon VP1 altered (GTG)
  source 1..2208: mol_type = other DNA
gtggctgccgacggttatctacccgattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 45 - DNA, 1800 bp, synthetic construct
  misc_feature 1..1800: note = AAV1; VP2; VP3
  source 1..1800: mol_type = other DNA
acggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 46 - DNA, 1605 bp, synthetic construct
  misc_feature 1..1605: note = AAV1; VP3
  source 1..1605: mol_type = other DNA
atggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 47 - protein, 736 aa, synthetic construct
  REGION 1..736: note = AAV1; VP1, VP2, VP3; startcodon VP1 altered (GTG)
VAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 48 - protein, 599 aa, synthetic construct
  REGION 1..599: note = AAV1; VP2, VP3
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 49 - protein, 534 aa, synthetic construct
  REGION 1..534: note = AAV1; VP3
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 50 - DNA, 19 bp, synthetic construct
  misc_feature 1..19: note = primer
  source 1..19: mol_type = other DNA
ttgacgcaaatgggcggta

SEQ ID NO: 51 - DNA, 23 bp, synthetic construct
  misc_feature 1..23: note = primer
  source 1..23: mol_type = other DNA
gatctgacggttcactaaacgag

SEQ ID NO: 52 - DNA, 26 bp, synthetic construct
  misc_feature 1..26: note = primer
  source 1..26: mol_type = other DNA
caagtatggcatctacaccaaagtct

SEQ ID NO: 53 - DNA, 25 bp, synthetic construct
  misc_feature 1..25: note = primer
  source 1..25: mol_type = other DNA
gcaatagcatcacaaatttcacaaa

SEQ ID NO: 54 - DNA, 29 bp, synthetic construct
  misc_feature 1..29: note = probe
  source 1..29: mol_type = other DNA
tgtgaactggatcaaggagaagaccaagc

SEQ ID NO: 55 - DNA, 25 bp, synthetic construct
  misc_feature 1..25: note = CMV probe with ZEN quencher at position 10
  misc_feature 10: note = ZEN-quencher - ZEN-quencher
  source 1..25: mol_type = other DNA
tatagacctncccaccgtacacgcc